(19) United States
(12) Patent Application Publication
(10) Pub. No.: US 2020/0102353 A1
(43) Pub. Date: Apr. 2, 2020

(54) HUMAN ENDOGENOUS RETROVIRAL PROTEIN

(71) Applicant: INSTITUT GUSTAVE ROUSSY, VILLEJUIF CEDEX (FR)

(72) Inventors: Odile HEIDMANN, PARIS (FR); Thierry HEIDMANN, PARIS (FR)

(21) Appl. No.: 16/713,930

(22) Filed: Dec. 13, 2019

Related U.S. Application Data
(63) Continuation of application No. PCT/EP2018/066837, filed on Jun. 22, 2018.

(30) Foreign Application Priority Data
Jun. 22, 2017 (EP) 17305775.3

(51) Int. Cl. C07K 14/005; C12N 7/00; C07K 16/10; G01N 33/574

(52) U.S. Cl. CPC C07K 14/005; C12N 7/00; C07K 16/1036; G01N 33/574; C07K 2317/569; C07K 2317/622; C07K 2317/54; C07K 2317/55; C12N 2740/10022

(57) ABSTRACT

Disclosed is human endogenous retroviral protein. This human endogenous retroviral protein is herein generally referred to as HEMO. The application relates more particularly to shed forms of the HEMO protein, more particularly to those shed forms, which are released in the circulating blood. The application also relates to products deriving from the shed forms of HEMO, such as antibodies, nucleic acid vectors and engineered cells, as well as to the medical or biotechnological applications of these shed forms or derived products, notably in the fields of placental development, fetus protection, cancer treatment and stem cell production.

### FIELD

The application relates to a human endogenous retroviral protein, more particularly to the human endogenous retroviral protein, which is coded by gene ERVMER34-1 (ORF LP9056). This human endogenous retroviral protein is herein referred to as Human Endogenous MER34 ORF, i.e., HEMO. The HEMO protein is conserved among the Boreoeutheria, more particularly among the Euarchontoglires and Laurasiatheria, more particularly among the primates.

The application relates more particularly to shed forms of the HEMO protein, more particularly to those shed forms of HEMO, which are released in the circulating blood. The application also relates to products deriving from the shed forms of HEMO, such as antibodies, nucleic acid vectors and engineered cells, as well as to the medical or biotechnological applications of these shed forms or derived products, notably in the field of placental development, fetus protection, cancer diagnostic, cancer treatment and stem cell production.

### BACKGROUND

Endogenous retroviral sequences represent approximately 8% of the human genome. These sequences (called HERVs for Human Endogenous Retroviruses) share strong similarities with present-day retroviruses, and are the proviral remnants of ancestral germ-line infections by active retroviruses which have thereafter been transmitted in a Mendelian manner. The >30 000 proviral copies found in the human genome can be grouped into about 80 distinct families, with most of these elements being non-protein-coding due to the accumulation of mutations, insertions, deletions and/or truncations. Yet, some retroviral genes have retained a coding capacity, and some of them have even been diverted by remote primate ancestors for a physiological role. This is the case of the so-called «syncytins», namely syncytin-1 and syncytin-2 in humans, which are retroviral envelope (env) genes captured 25 and 40 Mya, respectively, with a full-length protein-coding sequence, a fusogenic activity, and strong placental expression (Mi et al. 2000; Blond et al. 2000; Blaise et al. 2003; Lavialle et al. 2013). These genes have been demonstrated to be involved in placenta formation, with their fusogenic activity contributing to the formation of the syncytiotrophoblast at the materno-fetal interface, as a result of the syncytin-mediated cell-cell fusion of the underlying mononucleated cytotrophoblasts. Syncytins were thereafter identified in all placental mammals where they have been searched for, and their unambiguous role in placentation was demonstrated via the generation and characterization of knock-out mice. Syncytins are also present in Marsupials, where they are expressed in a short-lived placenta which is very transiently formed (a few days) before the embryo pursues its development in an external pouch.

Previous systematic searches for genes encoding endogenous retroviral Env proteins within the human genome have led to the identification of 18 genes with a full-length coding sequence (among which syncytin-1 and -2) (de Parseval et al. 2003; Villesen et al. 2004). These analyses have been performed using methods based on the search for characteristic motifs carried by retroviral Envs, which notably include, from the N- to the C-terminus,
a signal peptide,
a (canonical) furin cleavage site (R-X-R/K-R) between the surface (SU) and transmembrane (TM) subunits, with the latter carrying additional signatures including an immunosuppressive domain (ISD, 17 aa motif) which is also found in most oncoretroviruses,
a characteristic C-(X)6-7-C motif, and
a transmembrane hydrophobic domain anchoring the Env protein in the cell or virion membrane (de Parseval et al. 2005; Henzy et al. 2013).

The application relates to a human endogenous retroviral Env protein, which shares some but not all the structural features of the prior art human endogenous retroviral Env proteins, and which demonstrates unprecedented characteristics, more particularly unprecedented shedding characteristics.

### SUMMARY

The application relates to the human endogenous retroviral protein, which is coded by gene ERVMER34-1 (ORF LP9056). The protein is herein generally referred to as HEMO, which stands for Human Endogenous MER34 ORF.

The inventors demonstrate that the gene coding for the HEMO protein entered the genome of a mammalian ancestor more than 100 Mya, and that the HEMO protein is conserved among the Boreoeutheria, more particularly among the Euarchontoglires and Laurasiatheria, more particularly among the Euarchontoglires more particularly among the primates (cf. e.g., FIG. 10B).

By contrast to the prior art human endogenous retroviral Env protein, the human HEMO protein lacks the canonical furin cleavage site (it lacks the canonical R-X-R/K-R site, but shows an unusual CTQG site (at positions 352-355 in FIG. 1C)), and lacks the adjacent hydrophobic fusion peptide (cf. FIGS. 1A, 1B and 1C).

The HEMO demonstrates unprecedented characteristics, more particularly unprecedented shedding characteristics. Indeed, the ectodomain of the HEMO protein is cleaved by shedding, resulting in the release of HEMO ectodomain fragments in the circulating blood (cf. FIG. 13). The major soluble fragment produced by shedding of human HEMO extends from the first amino acid after the signal peptide up to (and including) the amino acid at position 432 or 433 (cf. FIG. 1C: from amino acid at position 25, 26 or 27, i.e., L, to amino acid Q at position 432 or amino acid R at position 433; cf. FIG. 13: cleavage site n° 1). Secondary soluble fragments include human HEMO fragments, which extend from the first amino acid after the signal peptide up to (and including) an amino acid at a position chosen from among positions 450-480 and 380-420 (cf. FIG. 13, cleavage sites n° 2 and 3) and 421-449.

The HEMO protein is highly expressed by stem cells and also by the placenta, resulting in an enhanced concentration in the blood of pregnant women. It is also expressed in some (human) tumors, thus providing a marker for a pathological state as well as, possibly, a target for immunotherapies.

The HEMO protein can be perceived as a “stemness” marker of the normal cell, and as a “target” for cancer immunotherapy.

The application relates more particularly to the shed forms of the HEMO protein, more particularly to the shed forms of HEMO, which are released in the circulating blood.

The application also relates to products, which derives from the shed forms of HEMO, such as antibodies, nucleic acid vectors and engineered cells.

The application also relates to the medical or biotechnological applications of these shed forms or derived products, notably in the fields of placental development, of fetus protection, of cancer treatment, and of stem cell production.

The application notably relates to means, which are useful for:
cancer diagnostic,
tumor typing,
cancer immunotherapy,
screening for therapeutic agents, e.g., screening for agents, which may be useful in the treatment of cancer (including palliation or prevention of cancer), or for agents, which may be useful in the treatment of a defect in placenta development (e.g., placental abruption, pre-eclampsia, eclampsia), or for agents, which may be useful in fetus protection (e.g., protection against viral or microbial infection),
purification of circulating cells (e.g., purification of tumoral circulating cells, or of circulating trophoblasts), and
production of induced pluripotent stem cells (from somatic cells).

### BRIEF DESCRIPTION OF THE FIGURES

The file of this patent contains at least one drawing executed in color. Copies of this patent with color drawings will be provided by the Patent and Trademark Office upon request and payment of the necessary fee.

Some of the figures, to which the present application refers, are in color. The application as filed contains the color print-out of the figures, which can therefore be accessed by inspection of the original application file.

FIGS. 1A, 1B, 1C and 1D: Structure of a canonical retroviral Env protein and characterization of the human HEMO Env.

(A) Schematic representation of a retroviral Env protein, delineating the SU and TM subunits. The furin cleavage site (consensus: R-X-R/K-R) between the two subunits, the C-X-X-C motif involved in SU-TM interaction, the hydrophobic signal peptide (purple), the fusion peptide (green), the transmembrane domain (red), and the putative immunosuppressive domain (ISD) (blue) along with the conserved C-X5/6/7-CC motif are indicated.

(B) Hydrophobicity profile of HEMO Env. The canonical structural features highlighted in A are positioned and shown in the color code used in A. The mutated furin site (CTQG) is shown as a dotted line.

(C) Amino acid sequence of the HEMO Env protein with the same color code.

(D) Retroviral Env protein-based phylogenetic tree with the identified HEMO-Env protein. The maximum likelihood tree was constructed using the full length SU-TM amino acid sequences from HERV Envs (including a HERV-K consensus), all previously identified syncytins and a series of endogenous and infectious retroviruses. The length of the horizontal branches is proportional to the average numbers of amino acid substitutions per site (see the scale bar at the lower left), and the percent bootstrap values obtained from 1,000 replicates are indicated at the nodes. ALV, avian leukemia virus; BaEV, baboon endogenous virus; BLV, bovine leukemia virus; Env-Cav1, syncytin-like Cavia porcellus Env1 protein; FeLV, feline leukemia virus; FIV, feline immunodeficiency virus; GaLV, gibbon ape leukemia virus; HERV, human endogenous retrovirus; HIV1, HIV type 1; HTLV-2, human T-lymphotropic virus type 2; mIAPE, Mus musculus intracisternal A-type particle with an env gene; JSRV, Jaagsiekte sheep retrovirus; KoRV, koala retrovirus; MMTV, murine mammary tumor virus; MoMLV, Moloney murine leukemia virus; MPMV, Mason-Pfizer monkey virus; PERV-A, porcine endogenous retrovirus; RD114, feline endogenous type-C retrovirus; ReV-A, Reticuloendotheliosis Virus type A, BIV, Bovine immunodeficiency Virus; Env-panMars, conserved Marsupial Env-2.

FIGS. 2A, 2B, 2C, 2D, 2E and 2F: Characterization of the HEMO env gene.

(A) Schematic representation of the HEMO gene locus on chromosome 4 (4q12, with the GRCh38 assembly coordinates of the Genome Reference Consortium).

Top: MER34-int consensus (Repbase) with putative gag, pro and pol retroviral ORFs indicated according to consensus aminoacid sequences. Dot lines delineate parts of the MER34 sequences found in the HEMO locus.

Middle: the HEMO gene locus (11 kb) is located between the RASL11B gene (^{˜}120 kb 5′) and the USP46 gene (^{˜}120 kb 3′). HEMO env ORF is shown as an orange box, repetitive sequences identified on the Dfam.org web site, are shown as different colored boxes with the sense sequences above and anti-sense sequences below the line. Of note, the gene is part of a MER34 provirus which has kept only degenerate pol sequences (mostly in opposite orientation), a truncated putative 3′LTR (MER34-A) and no 5′LTR. No other MER34 sequences are found 100 kb apart from the gene. A CpG Island (chromosome 4:52750911-52751703), detected by the EMBOSS-newcpgreport software, is indicated as a green box.

Bottom: intron-exon structure predicted from NCBI and RNA transcripts: exons found in placental RNA, as determined by 5′ and 3′ RACE experiments, are indicated with the main E1-E2-E4 spliced env subgenomic transcript below. Start site nucleotide sequence (ACTTC . . . ) and acceptor splice site for the HEMO env ORF are depicted. Arrows specify qRT-PCR primers (Table 4).

(B) Real-time qRT-PCR analysis of the HEMO transcripts in a panel of 20 human tissues and 16 human cell lines. Transcript levels are expressed as percent of maximum and were normalized relative to the amount of housekeeping genes (see Methods). Placenta values are the means of 12 samples from 1st trimester pregnancies and other tissues are from a commercial panel (Zyagen).

(C) CpG island promoter sequence around the Transcription Start Site (+1, ACTTC in red), with CG dinucleotides in green highlighted. Exon1 and exon2 are boxed. Nucleotide sequences in grey represent primer sequences used for amplification of the two fragments (I and II, vertical bars on the left) analyzed after bisulfite treatment (panel E).

(D) Luciferase assay of the CpG Island promoter.

Top. Schematic representation of the promoter-luciferase constructs with the CpG island (in green) containing exons E1 and E2. E2 was shortened at its 3′ end, 28 bp upstream of the donor splice site, to limit splicing out of the luciferase gene.

Bottom. Promoter sequences in each pGL3 construct are indicated as white boxes, with coordinates relative to the +1 Transcription Start Site of the gene. Control (none) corresponds to the basic pGL3 vector, with no inserted sequence. Promoter activity, expressed in light unit (LU), was determined using the Luciferase reporter assay, in lysates from 293T cells transfected with the pGL3 vectors. The plotted data are the average from three independent experiments.

(E) Methylation status of the HEMO promoter region as revealed by bisulfite treatment of the genomic DNA from cell lines not expressing (293T and BeWo cells) or expressing (iPSC and CaCo-2) the HEMO gene, and PCR amplification of fragments I (26 CpG) and II (33 CpG) delineated in panel C. The graph represents the sequencing of 10 clones for each PCR-amplified fragment, with methylated (black circle) and un-methylated (white circle) CpG indicated.

(F) Effect of DNA demethylation on expression of the HEMO gene. HEMO gene transcription levels were detected by RT-qPCR and normalized to the housekeeping gene RPLPO, in cell lines (293T, BeWo) untreated (DMSO alone) or treated with 0.1 to 5 μM of 5-Aza-2′-deoxycytidine (Aza-dC) for 3 days. Data are presented as the mean+/−SEM. Asterisks (*) indicate values significantly different from that obtained with untreated cells (unpaired two-tailed t test; *, P<0.05; ***, P<0.001).

FIG. 3: Immunofluorescence analysis of HEMO protein expression in transfected HeLa cells.

Cells (HeLa) were transfected with the phCMV-HEMO expression vector (or an empty vector as a negative control), fixed, permeabilized (upper panel) or not permeabilized (lower panel), and stained for HEMO protein expression using a specific anti-HEMO polyclonal antibody (see Methods). Upper panel: Specific staining of the phCMV-HEMO transfected cells versus empty vector transfected cells. Lower panel: Successive confocal images demonstrate cell surface localization of the protein.

FIGS. 4A, 4B and 4C: Characterization of the shed HEMO protein.

(A) Detection of the shed HEMO Env protein by Western Blot analysis. (Left) Detection of the syncytin-1 protein with the anti-Env-W polyclonal antibody in the cell lysate of phCMV-Env-W transfected 293T cells. (Center and Right) Detection of the two forms of the HEMO protein (full-length SU-TM and Shed Env) with the anti-HEMO polyclonal antibody in the cell lysate and supernatant of phCMV-HEMO transfected 293T cells (Center) and first trimester placental tissue and placental blood (Right; matched representative samples from the same individual); samples were treated (+) or not (−) with PNGase F.

(B) Mass spectrometry determination of the N- and C-termini of the shed HEMO protein. Protein coverage of the Shed Env form, purified from the supernatant of phCMV-HEMO transfected 293T cells, is shown in green characters after trypsin proteolysis and mass spectrometric characterization of the resulting peptides, and with underlined characters for chymotrypsin proteolysis (see Methods). The HEMO N- and C-termini are indicated by a capital letter, and (*) indicates the positions of the stop codons in the mutants generated and analyzed in C.

(C) Migration pattern of the mutant HEMO forms, analyzed as in A. Left: schematic representation of the HEMO protein with the stop codons of the generated mutants positioned, together with that of the mutant with a reconstituted furin site (H-fur+, with a RTKR furin site). Right: Supernatant of 293T cells transfected with the expression vectors for the wild type (WT) and the mutant HEMO plasmids, analyzed after PNGase F treatment, SDS gel electrophoresis, and western blot as in A.

FIG. 5: Inhibition of HEMO release in the supernatant of transfected cells.

Western blot analysis of cell lysate and supernatant of 293T cells transfected with the phCMV-HEMO expression vector, using the polyclonal anti-HEMO polyclonal antibody (see Methods). Cells were treated for 3 days, with the indicated doses of the ADAM and MMP chemical inhibitors Batismastat, Marismastat, and GM6001 or DMSO alone. Anti-γ-tubulin antibody was used as a control of cell lysate protein loading. The full-length HEMO-protein (SU-TM) and the secreted form (Shed Env) are indicated by arrowheads.

FIG. 6: Release of the HEMO protein in the peripheral blood during pregnancy.

Western blot analysis of purified blood samples, with the polyclonal anti-HEMO antibody (upper panel) and anti-hCG-beta antibody (lower panel). The shed HEMO protein is detected in the placental blood from 1st trimester pregnancy (T1), and from peripheral blood of men (M), non-pregnant women (F), and pregnant women from 1st (T1), 2nd (T2) and 3rd (T3) trimesters. Bands observed at both higher and lower MW might correspond to minor alternatively processed/shed forms of the HEMO protein.

FIGS. 7A, 7B and 7C: Immunohistochemical detection of the HEMO protein in formalin-fixed tissues of first trimester human placenta.

(A) Schematic representation of the feto-placental unit with an enlarged anchored villus bathed by maternal blood and displaying the syncytiotrophoblast (ST) layer, the underlying mononucleated cytotrophoblasts (CT) and the invading extravillous cytotrophoblasts (EVT).

(B) Serial sections of multiple placental villi and chorionic membrane stained with a control IgG2a mouse isotype (left) or with the anti-HEMO monoclonal antibody 2F7 (right, CNCM I-5211). Magnification 4×.

(C) Enlarged views of the 4 domains delineated in B: placental villi with preferential CT (1-2) and EVT (3) staining; chorionic membrane with CT staining (4). Magnification 60×.

FIGS. 8A, 8B, 8C and 8D: Expression of the HEMO gene during development by in silico RNA-seq analysis.

A-C: In silico analysis of three panels of RNA-seq data, for HEMO, syncytin-1 (env-W) and -2 (env-FRD), GCM1 (Glial Cells Missing homolog 1, a specific placenta-expressed gene) and OCT4 (highly expressed in stem cells). RNA-seq raw data were screened with the coding part of each gene and hits were reported in log scale, per kilobase of screened sequence and after normalization with two house-keeping genes, RPLPO and RPS6.

(A) panel of 124 single-cell RNA-seq of human preimplantation embryos and embryonic stem cells (similar patterns were obtained from data in ref. (30), that covered the oocyte to morula stages).

(B) panel of 7 samples of normal placental tissues.

(C) panel of 28 RNA-seq samples from the reprogramming of human CD34+ cells (NT) to iPS cells and from human ES cell lines.

D: Western Blot analysis of WGA-purified placental blood (first trimester pregnancy) and of WGA-purified supernatant of confluent iPSC-cloneN, (grown an extra-36 h without serum and concentrated 20×). Samples were treated with PNGase F. The shed HEMO form is detected using the polyclonal anti-HEMO antibody.

FIGS. 9A, 9B, 9C and 9D: Microarray analysis of HEMO expression within normal tissues and tumor samples.

(A, B) Box plot representations of normalized values obtained for HEMO gene expression, extracted from the E-MTAB62 dataset (on a logarithmic scale). Original tissue categories were adjusted to group together samples from the same biological source, keeping the major groups described by the authors: normal tissues (A), and tumor samples (B).

(C) Box plot representation of normalized values obtained from an enlarged ovarian tumor sample, extracted as raw.CEL files from various AE and GEO studies (see Methods). Values for normal ovarian tissues were included, as control, in the normalization process. Tumoral ovarian histotypes correspond to 60 Clear Cell Carcinoma, 96 Endometrioid, 34 Mucinous and 289 Serous tumoral samples. (Wilcoxon's rank sum test; **, P<0.01).

(D) Immunohistochemical analysis using the 2F7 monoclonal antibody (CNCM I-5211) specific for the HEMO protein (or a control isotype), of formalin-fixed normal ovarian tissues (left) and ovarian Clear Cell Carcinoma (right, at two magnifications).

FIGS. 10A, 10B and 10C: Sequence conservation and purifying selection of the HEMO gene in simians.

(A) Syntenic conservation of the HEMO locus in mammalian species. The genomic locus of the HEMO gene, on human chromosome 4, along with the surrounding RASL11B and USP46 genes (275 kb apart), was recovered from the UCSC Genome Browser together with the syntenic loci of the chimpanzee, macaque, marmoset, tarsier, mouse lemur, colugo, mouse, guinea pig, rabbit, hedgehog, cow, horse, dog, cat, elephant, tenrec, armadillo and opossum genomes; exons of the RASL11B and USP46 genes and the sense of transcription (arrows) are indicated. Exons of the HEMO gene (E1 to E4) are shown on an enlarged view of the 15 kb HEMO locus, together with the homology of the syntenic loci (analyzed using the MultiPipMaker alignment-building tool). Homologous regions are shown as green boxes, and highly conserved regions (more than 100 bp without a gap displaying at least 70% identity) are shown as red boxes. Sequences with (+) or without (−) a full-length HEMO ORF are indicated on the right (nr: not relevant).

(B) HEMO-based maximum likelihood phylogenetic tree was determined using nucleotide alignment of the HEMO gene, inferred with the RAxML program. The horizontal branch length and scale indicate the percentage of nucleotide substitutions. Percent bootstrap values obtained from 1.000 replicates are indicated at the nodes. Double-entry table for the pairwise percentage of amino acid sequence identity (lower triangle) and the pairwise value of dN/dS (upper triangle) between the HEMO gene from the various simian species listed on the phylogenetic tree to the left and listed in the same order in abbreviated form at the top. A color code is provided for both series of values. (OWM: Old World Monkeys; NWM: New World Monkeys; AGM: African Green Monkey; m.: monkey).

(C) Conservation of HEMO shedding in simians illustrated by Western blot analysis of 293T cells transfected with expression vectors for the indicated simian HEMO genes, or for the human HEMO mutant with a consensus furin site (H-fur+). Cell lysates and supernatants were harvested and treated with PNGase F prior to Western blot analysis with the polyclonal anti-HEMO antibody. The entire SU-TM HEMO protein is the main form observed in cell lysates, whereas the shed and the free SU form (for the NWM genes with a furin site and the H-fur+ mutant) are mainly observed in the supernatants.

FIGS. 11A and 11B: Aligned amino acid sequences of the simian HEMO proteins. The characteristic domains are delineated, with the putative proteolytic furin cleavage site (RXKR, in black) between the SU and TM subunits, the signal peptide (in purple) and the CWLC motif (CXXC, black) in the SU subunit, the immunosuppressive domain (ISD, blue), C6XCC sequence (black) and the transmembrane domain (red) in the TM subunit. Dots indicate amino acid identity and hyphens codon deletions. HUM (human); CPZ (chimpanzee); GOR (gorilla); ORA (orang-outan); GIB (gibbon); MAC (macaque); BAB (baboon); AGM (African Green Monkey); COL (Colobus); LAN (langur); RHI (rhinopithecus); MAR (marmoset); SQM (squirrel monkey); SPI (spider monkey); SAK (saki).

FIGS. 12A, 12B, 12C, 12D and 12E: Characterization of the marsupial env-panMars gene and protein.

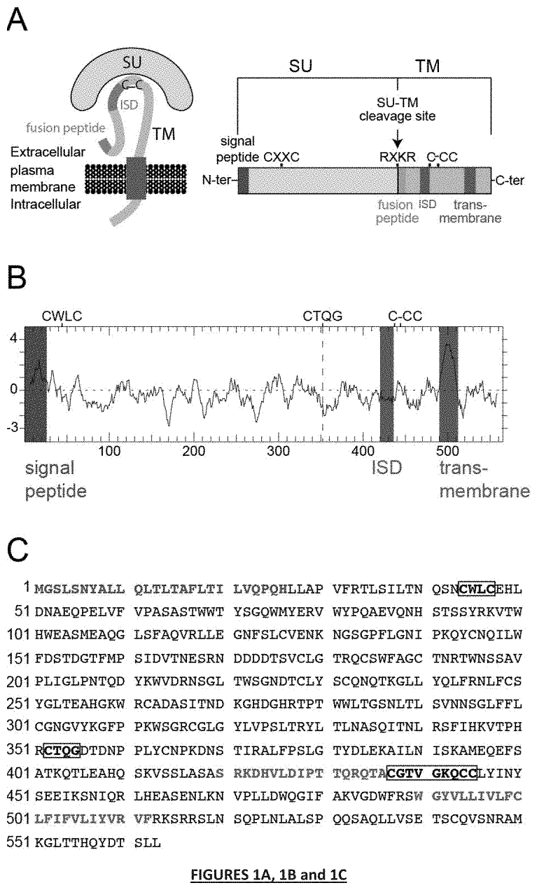

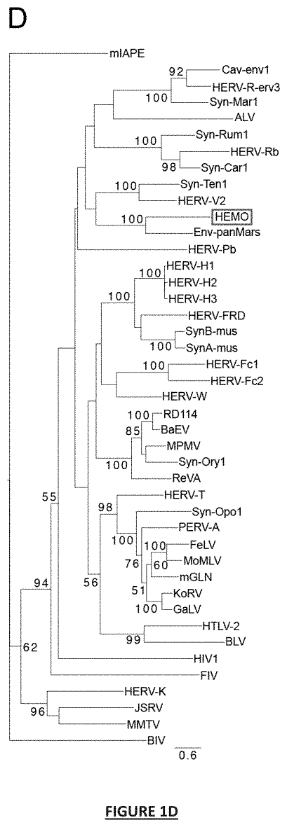

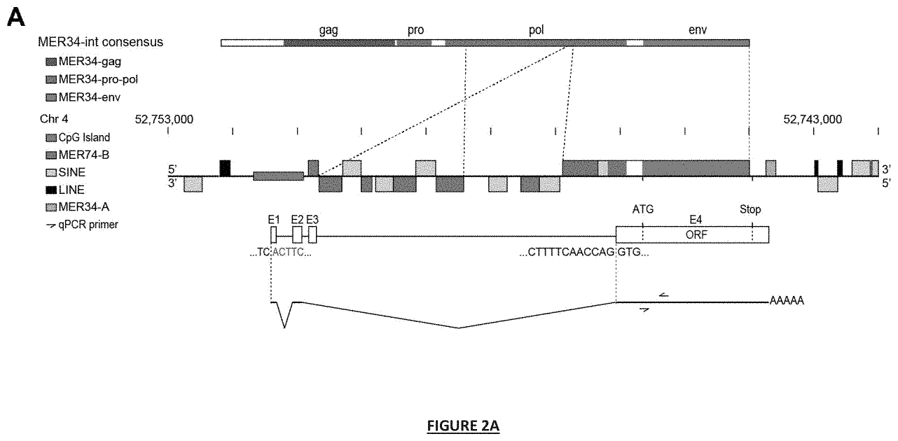

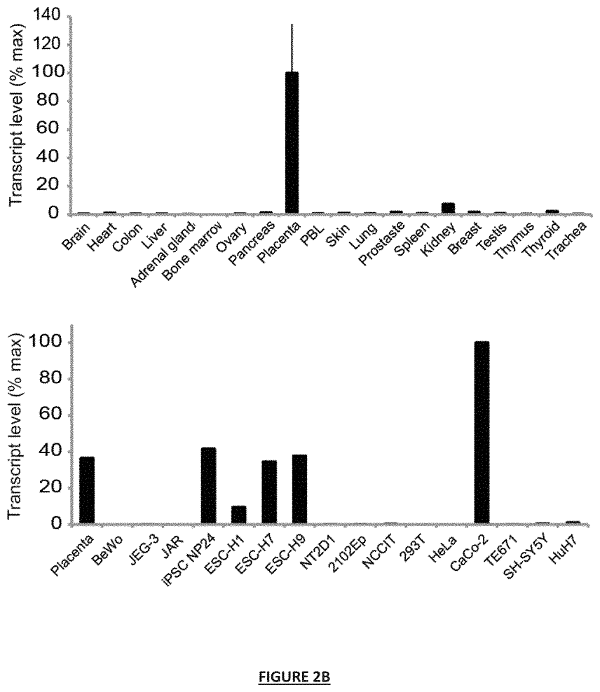

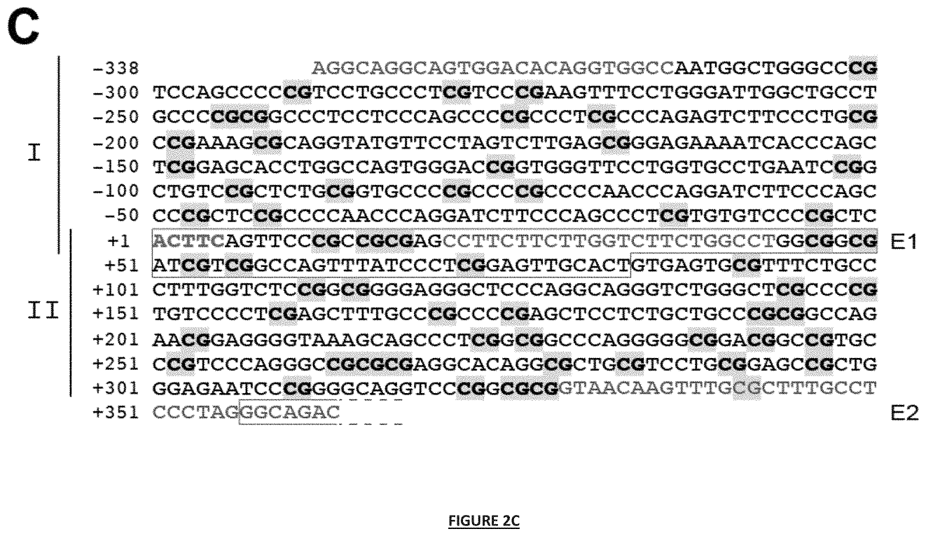

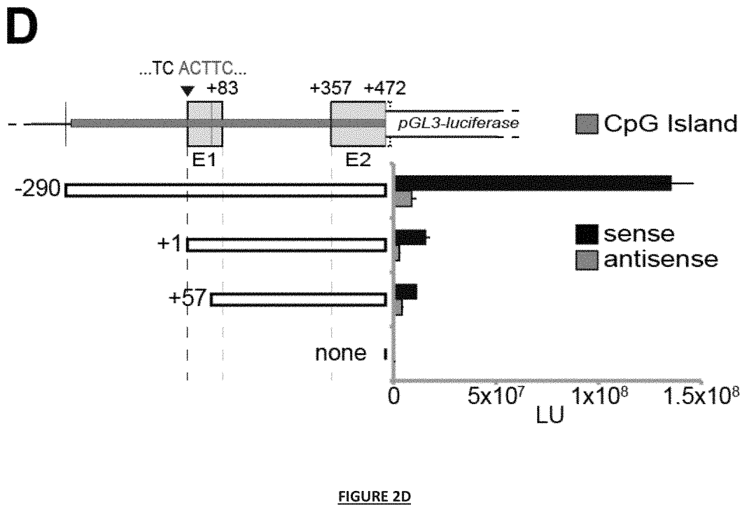

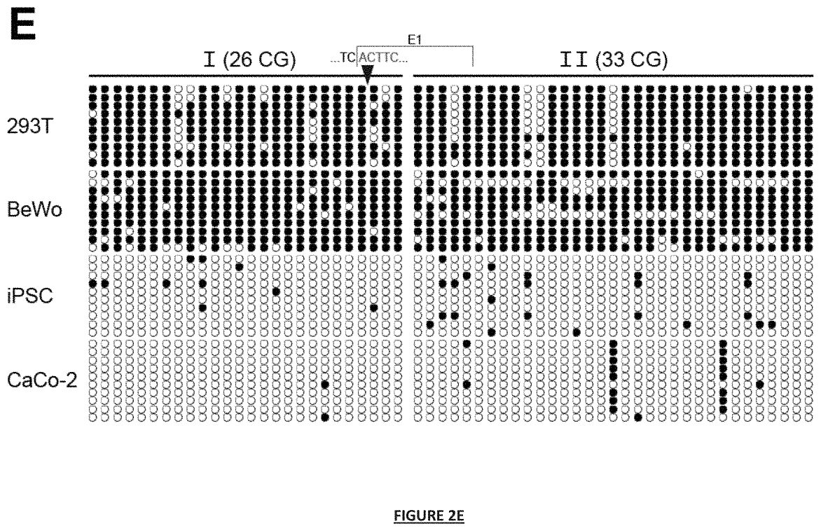

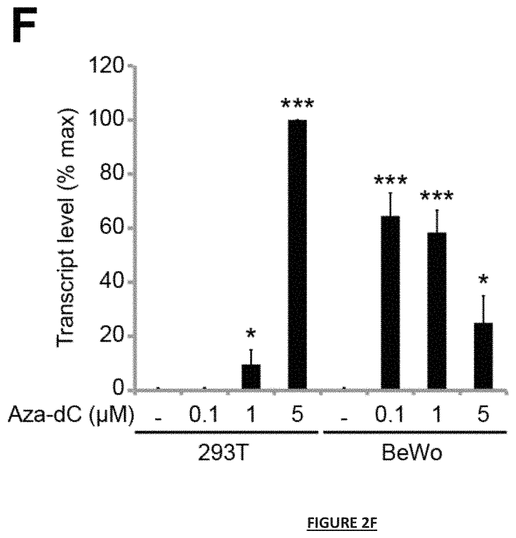

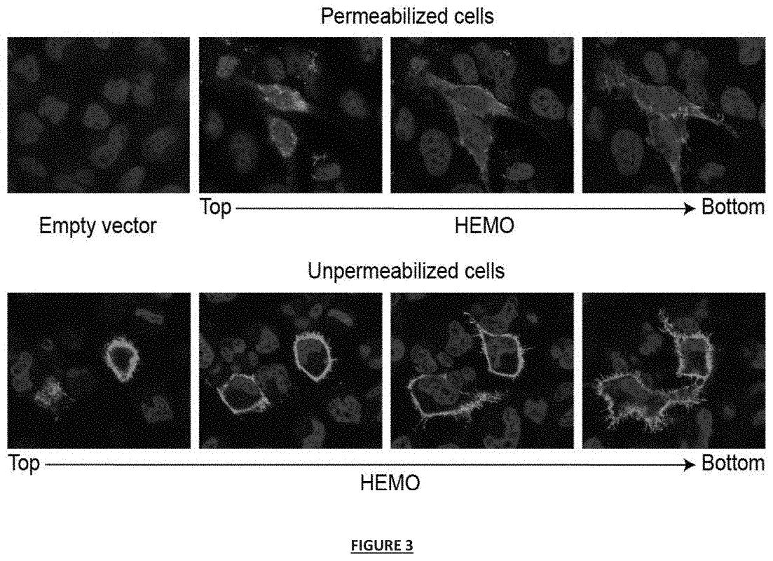

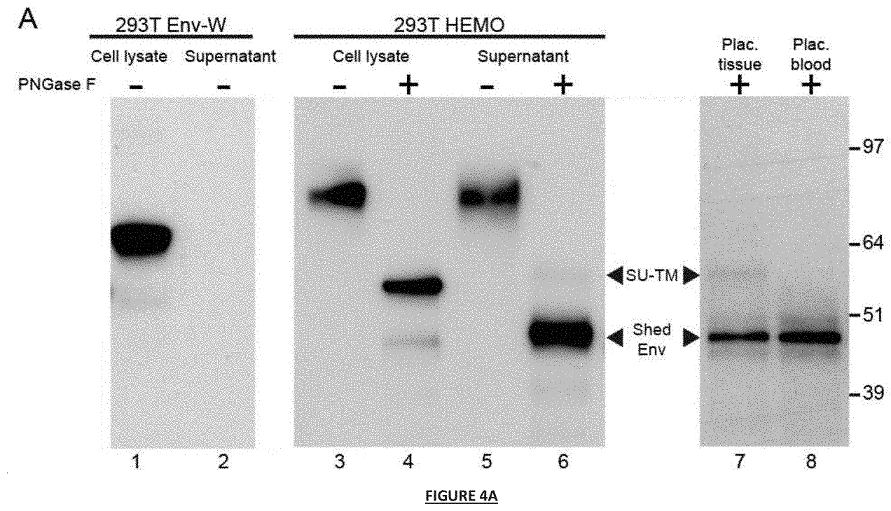

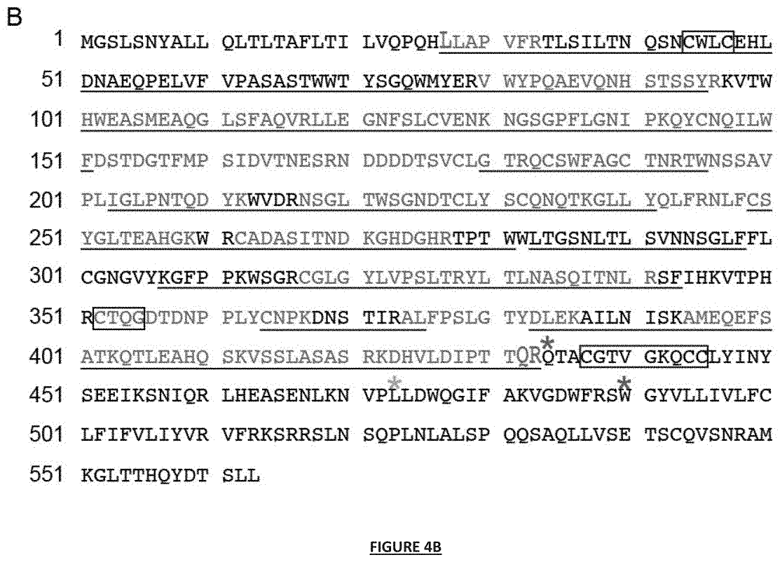

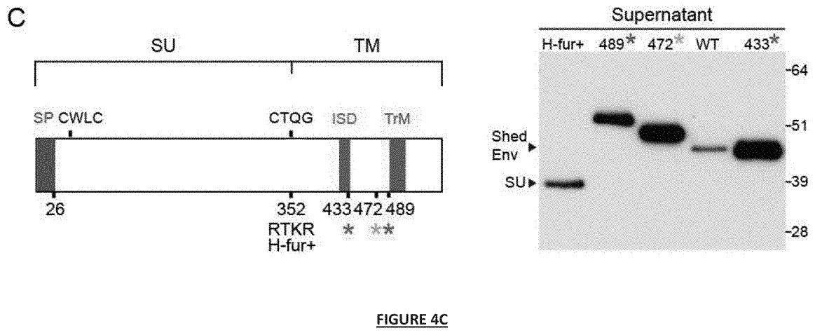

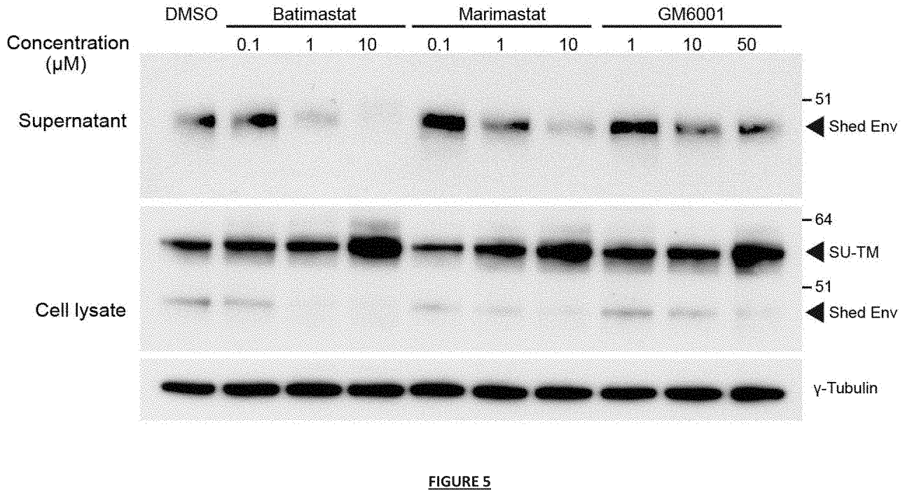

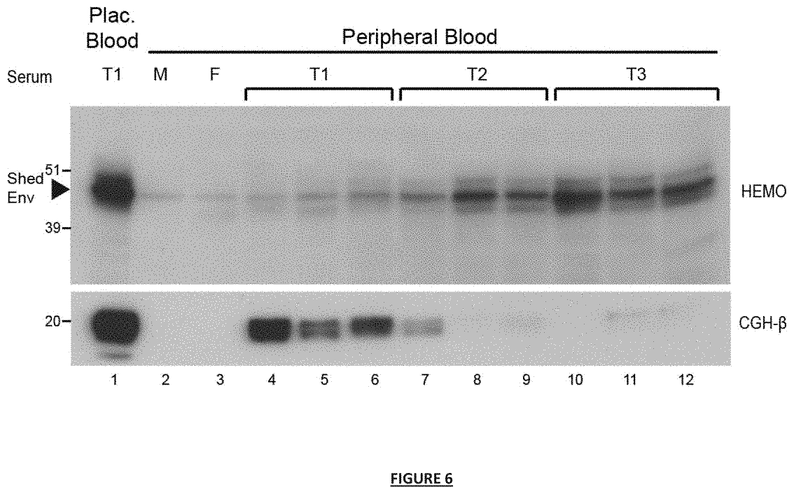

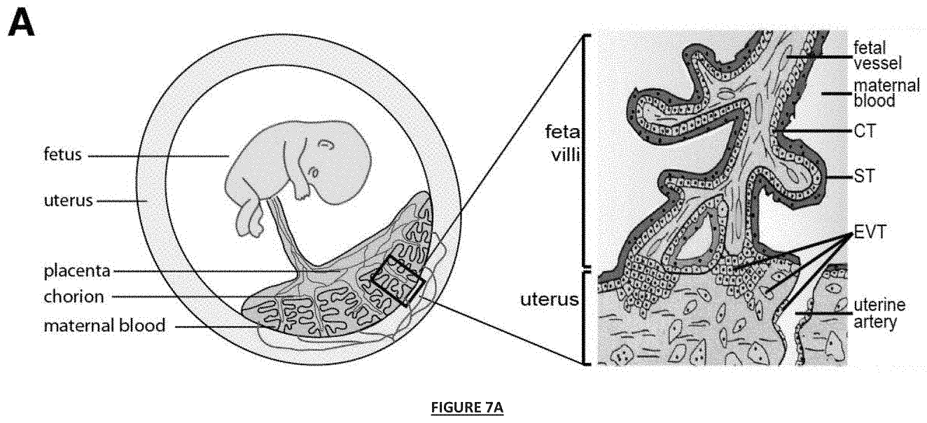

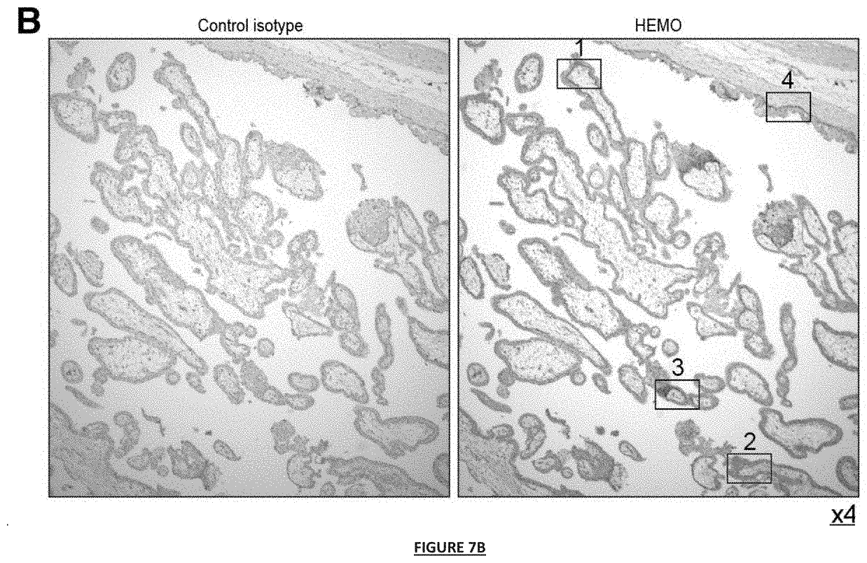

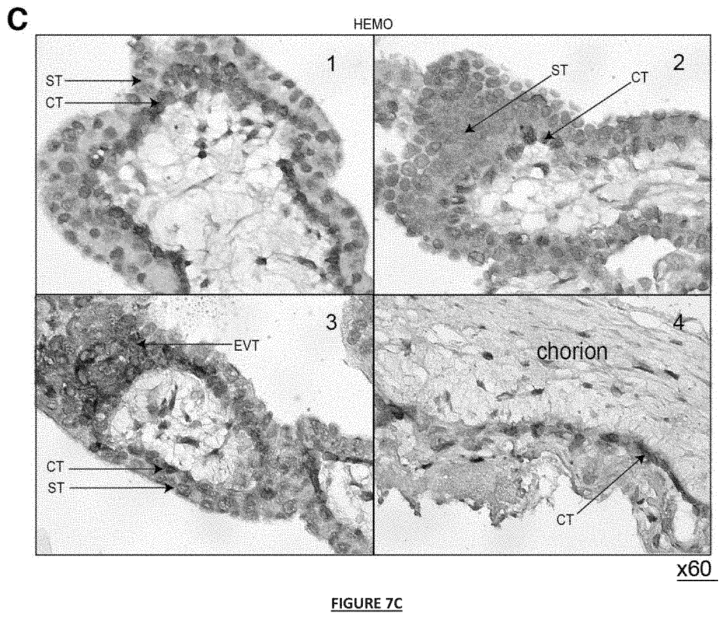

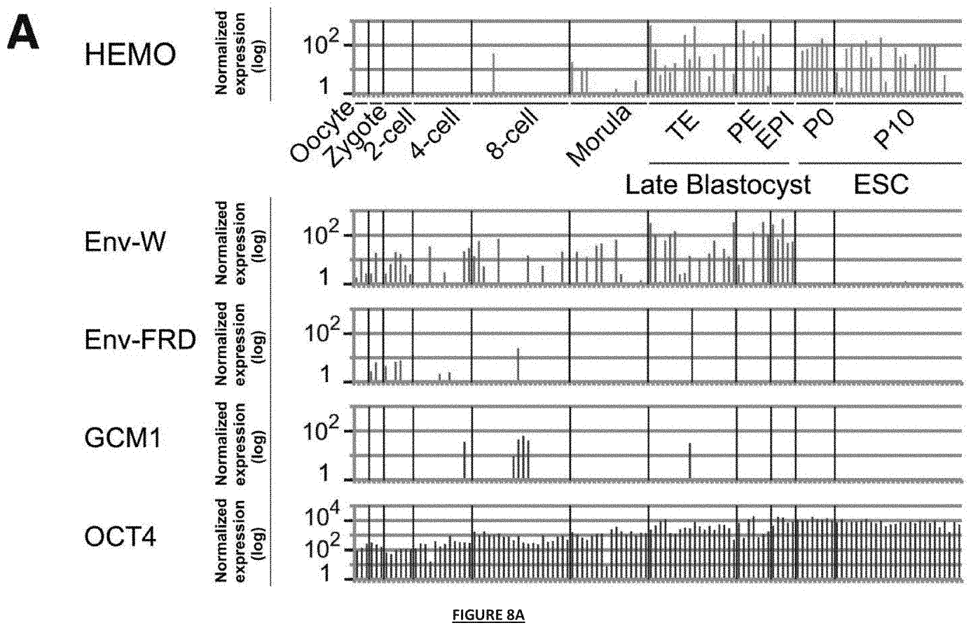

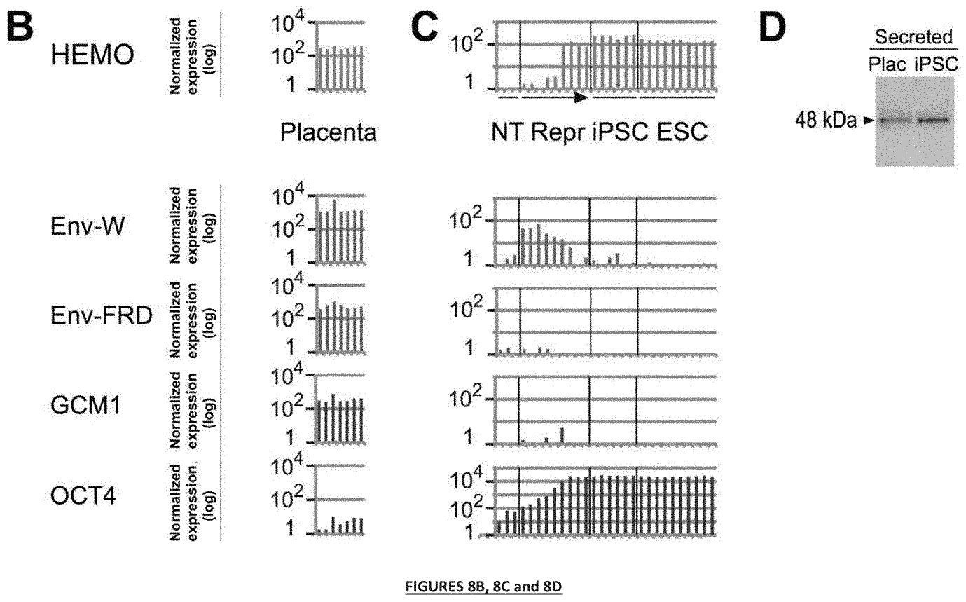

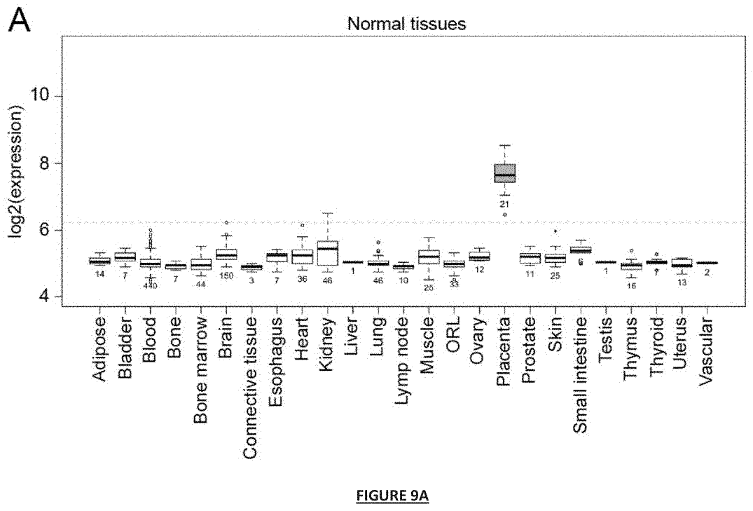

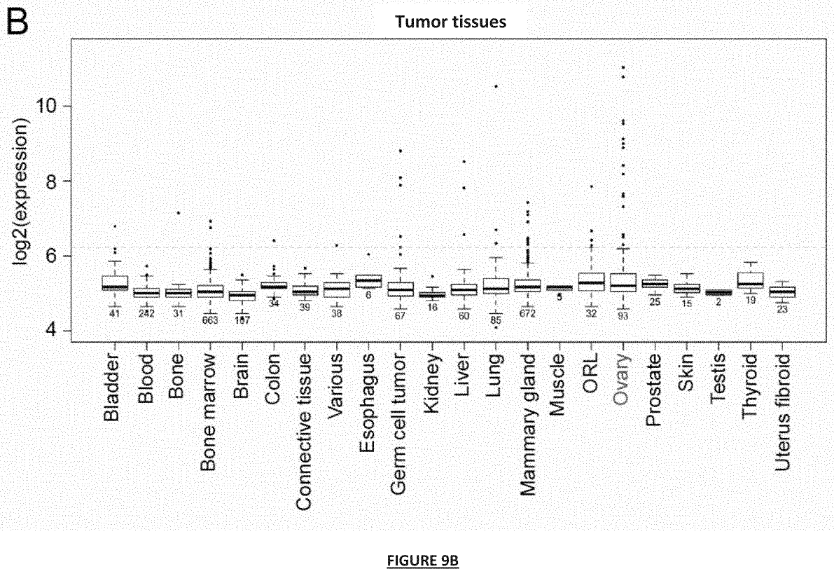

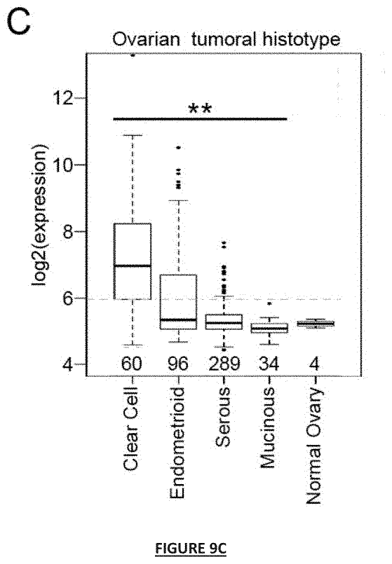

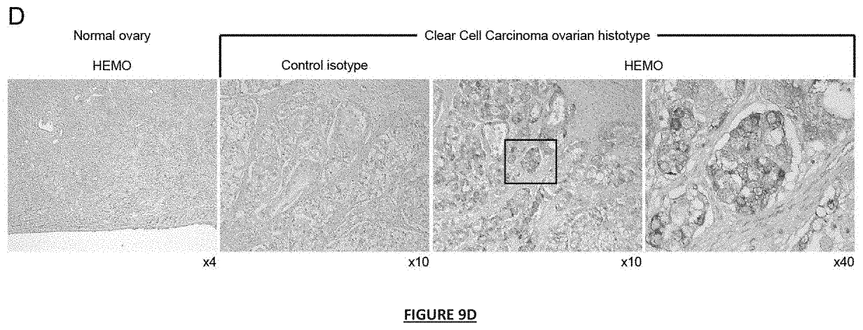

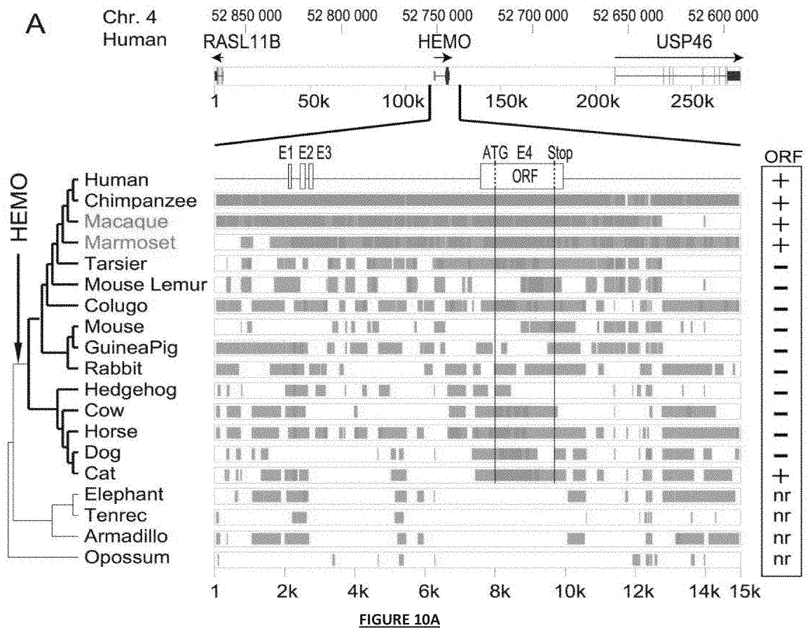

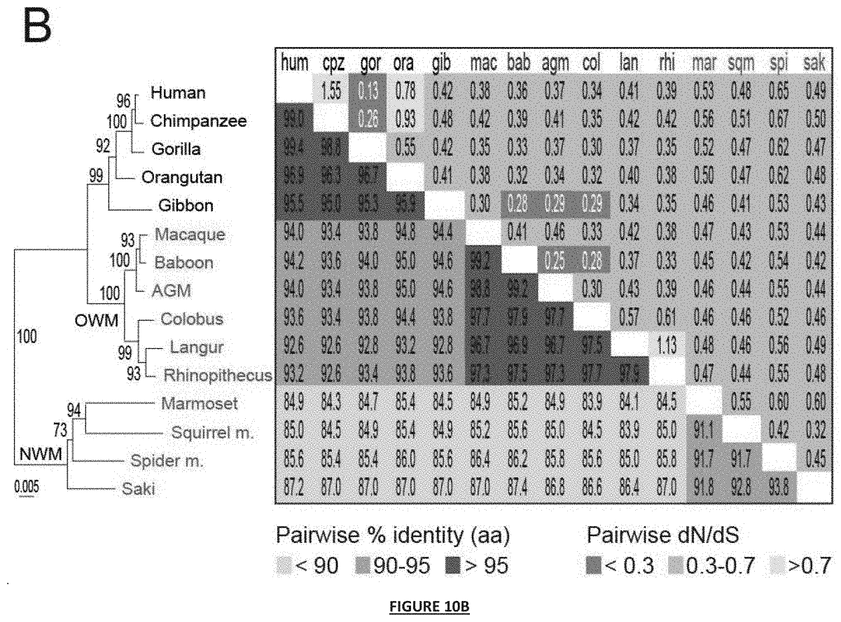

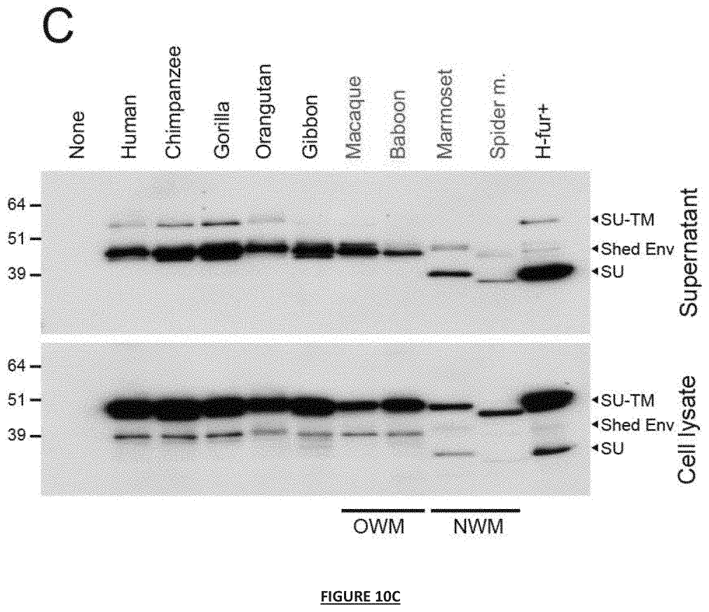

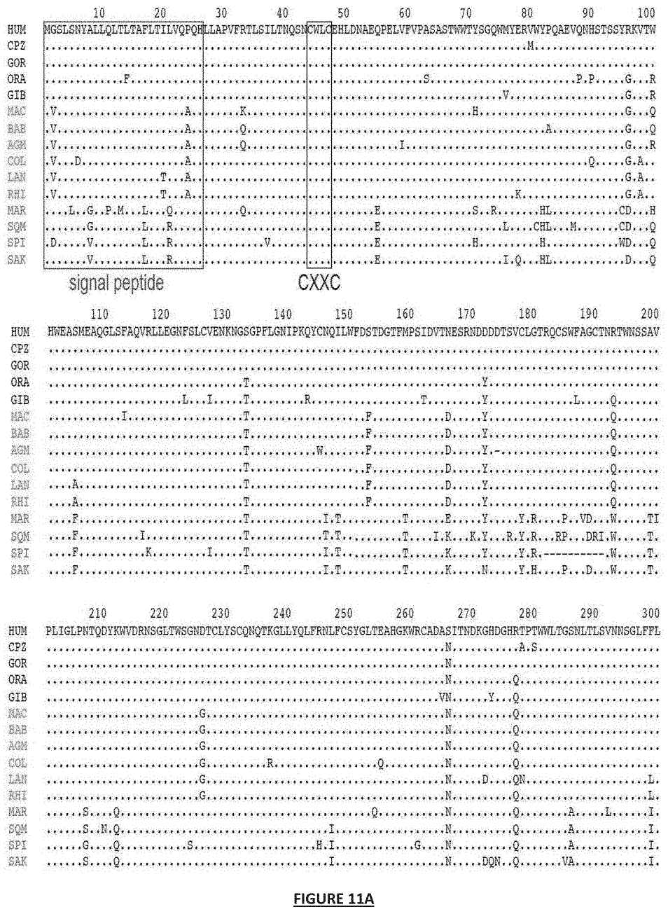

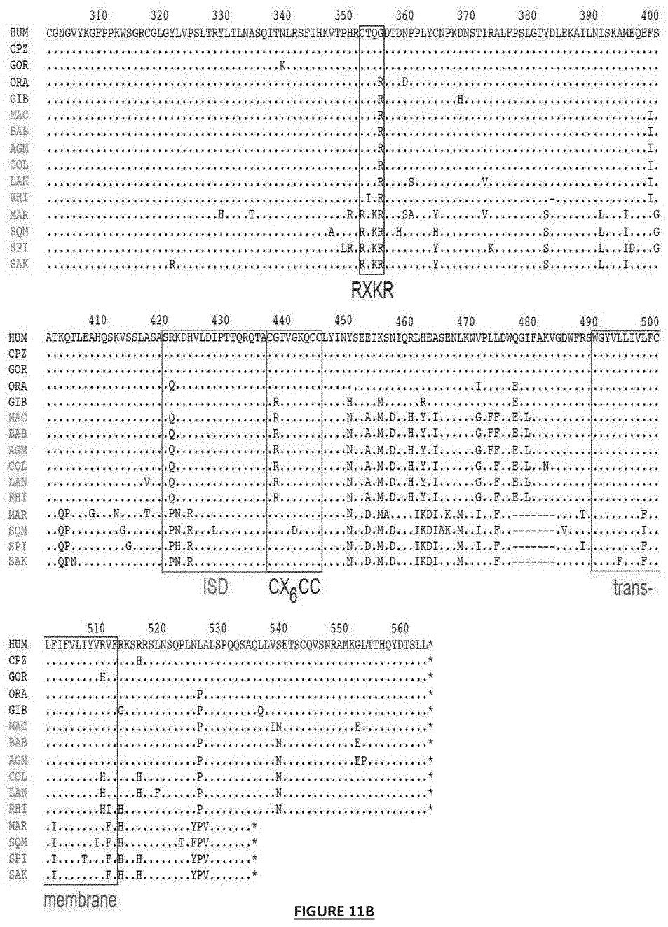

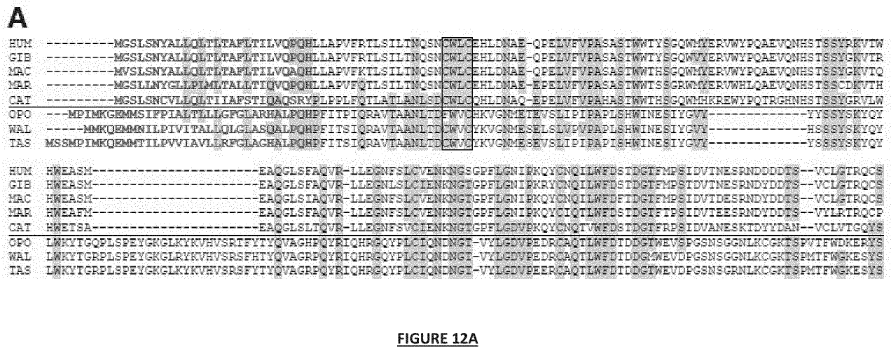

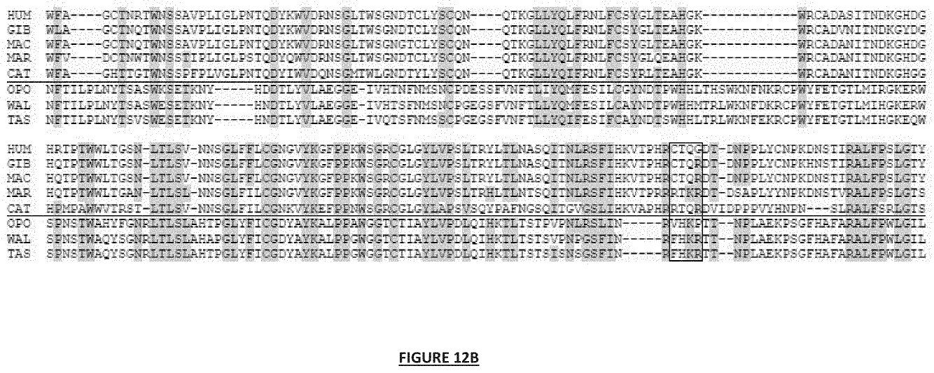

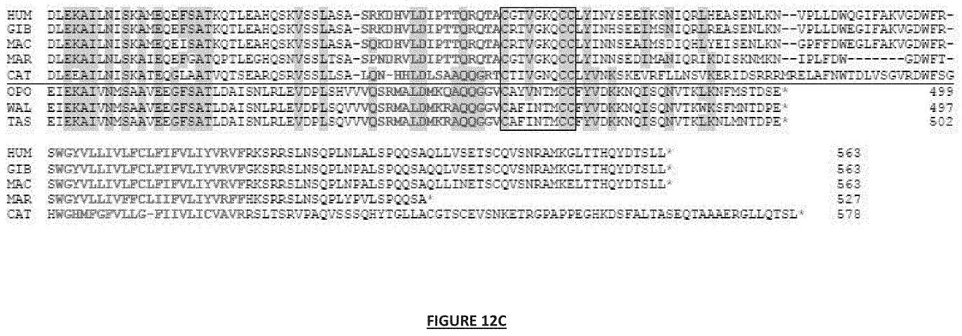

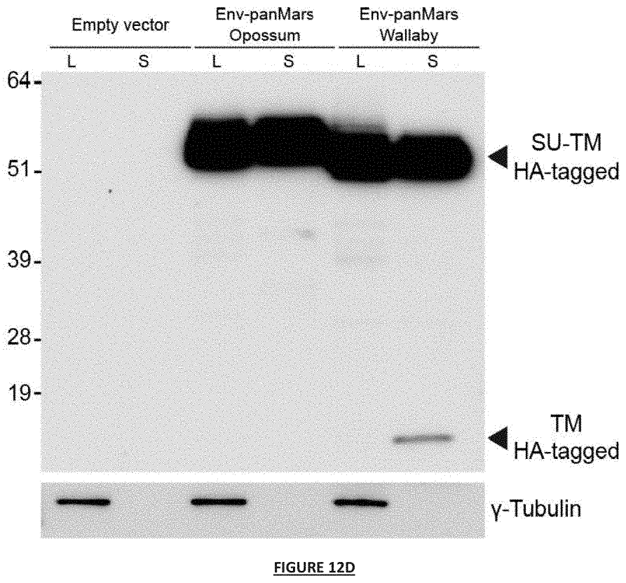

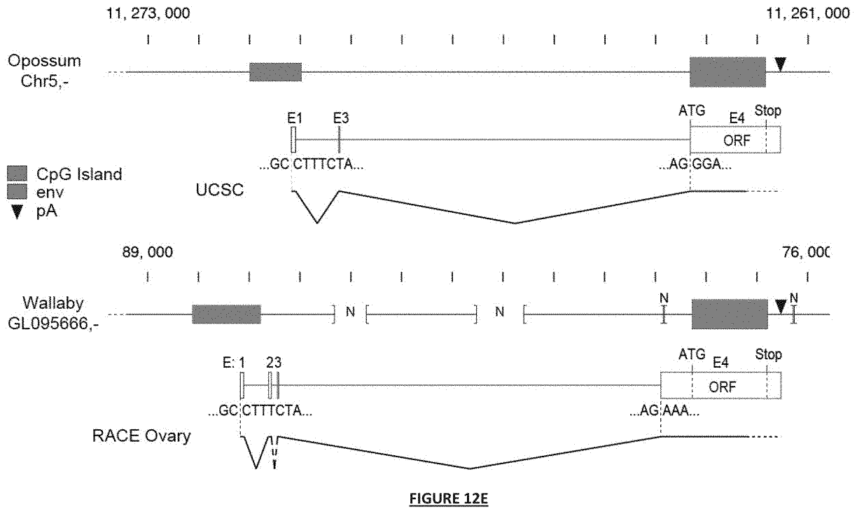

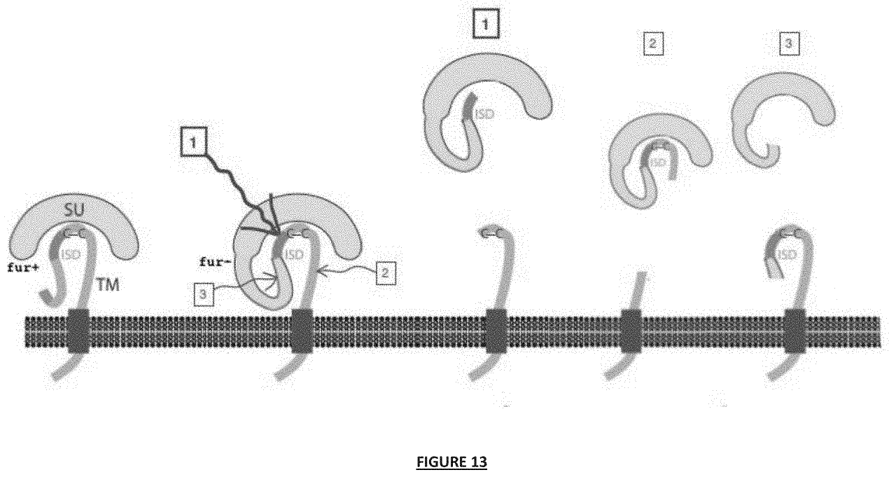

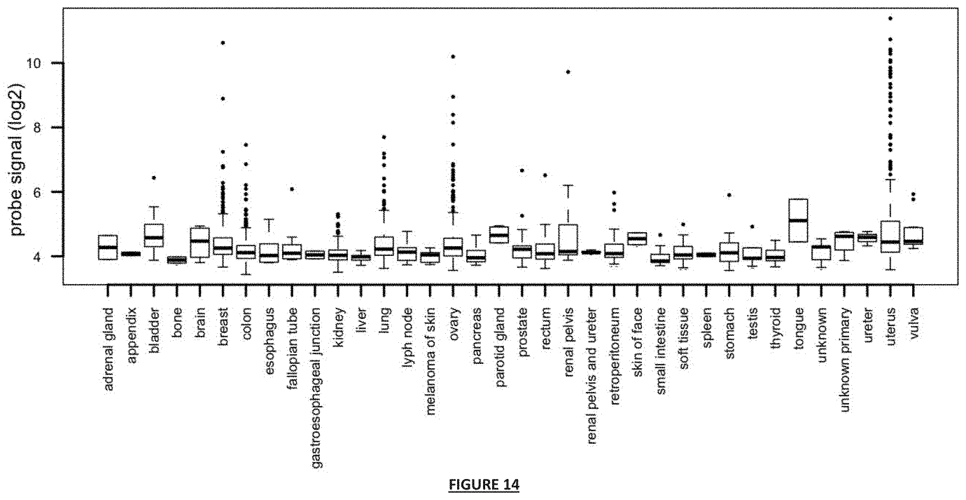

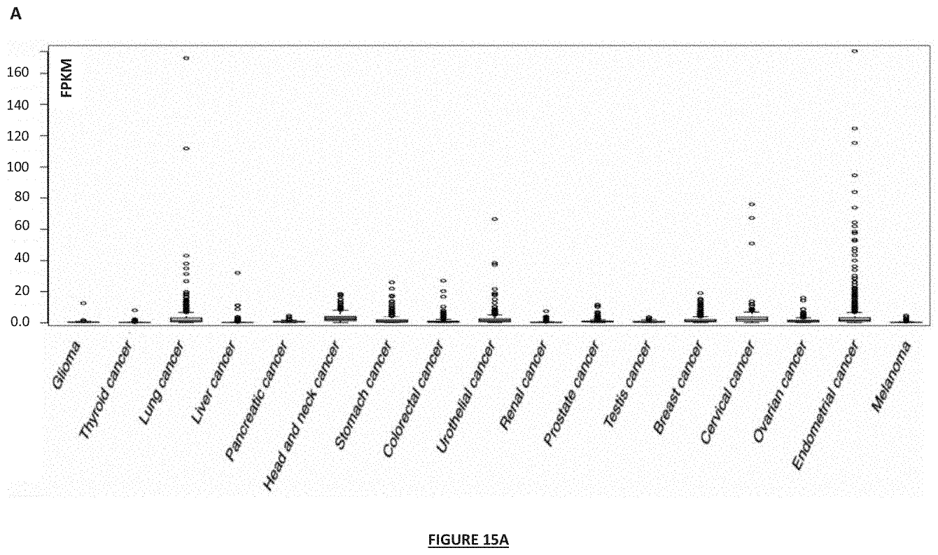

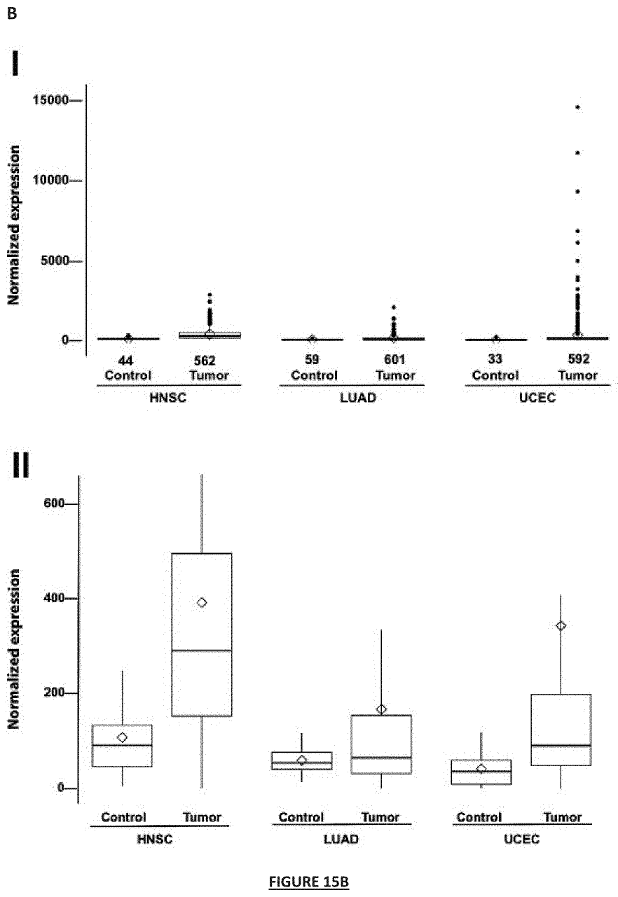

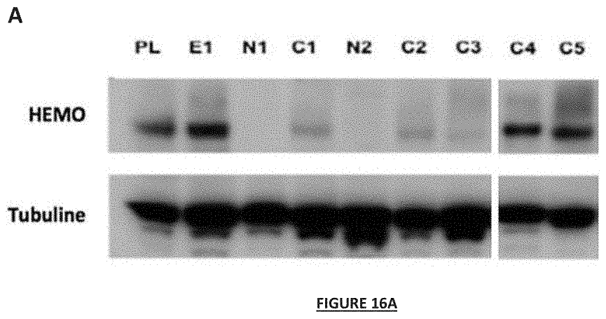

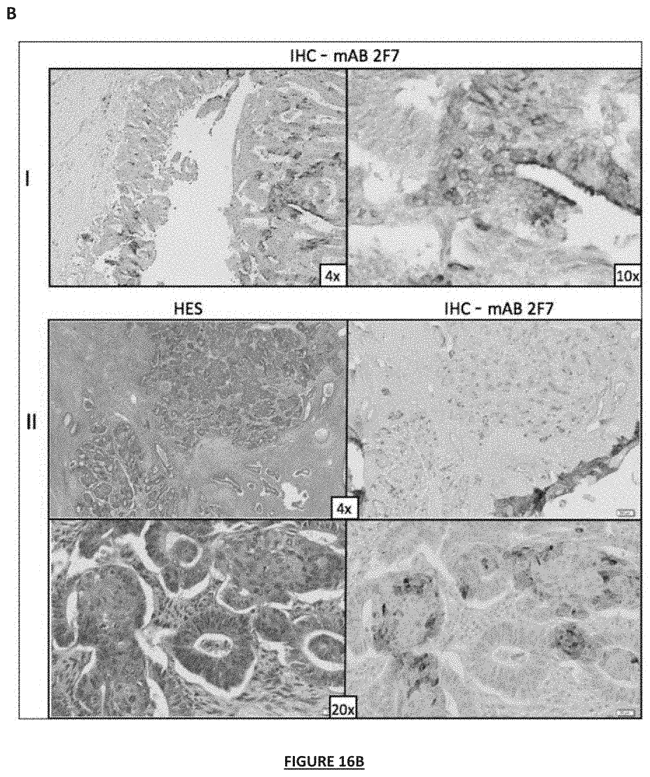

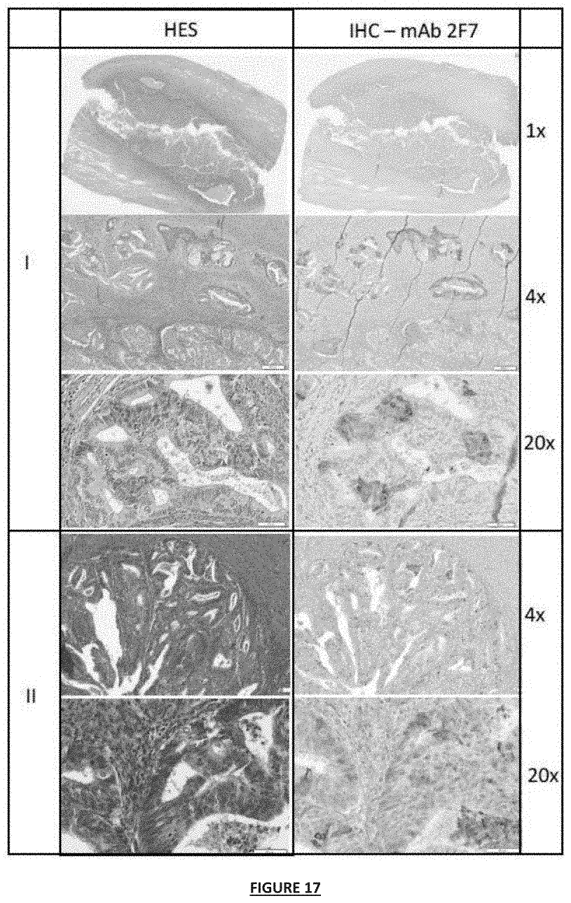

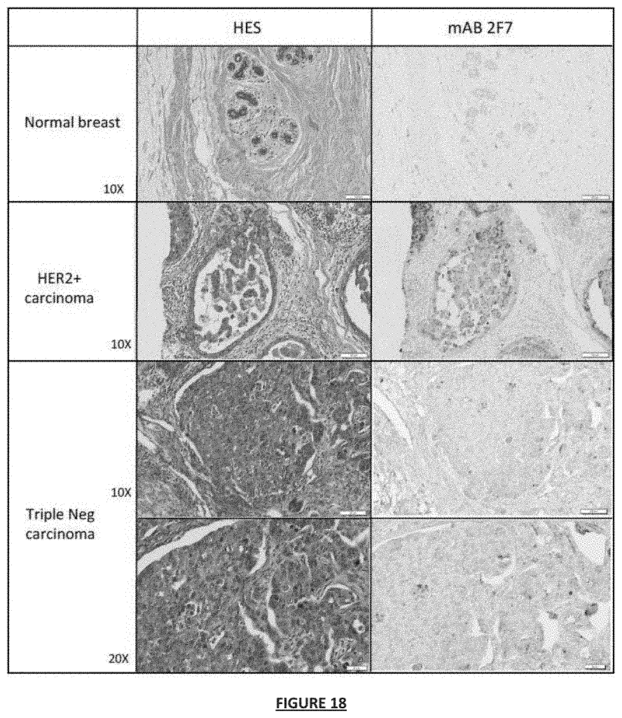

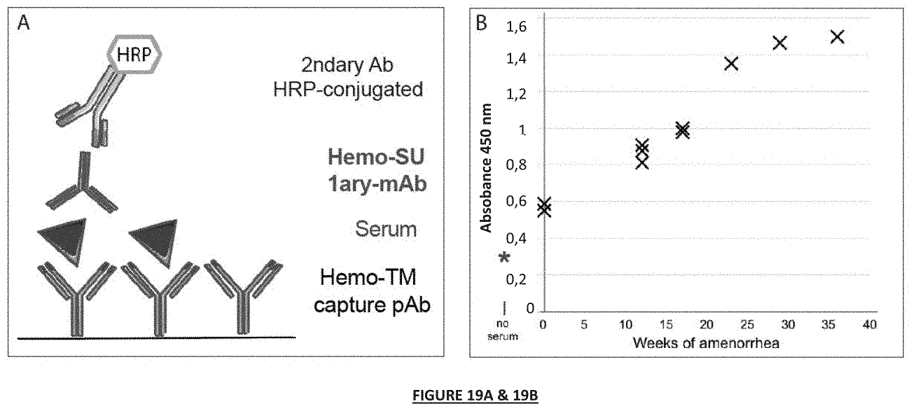

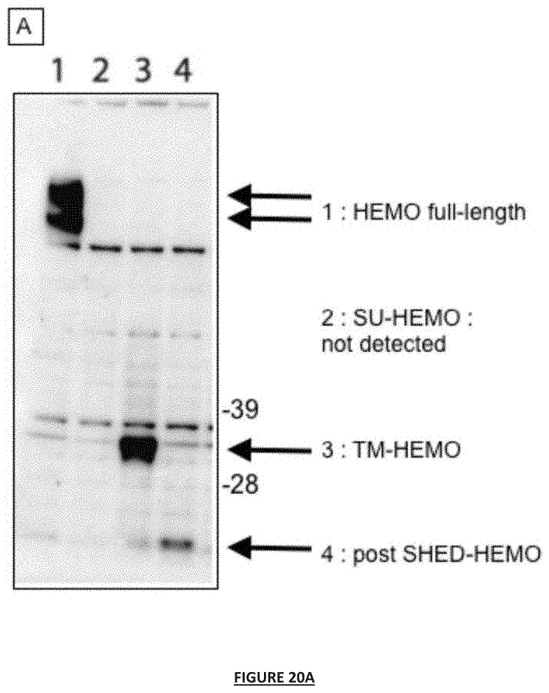

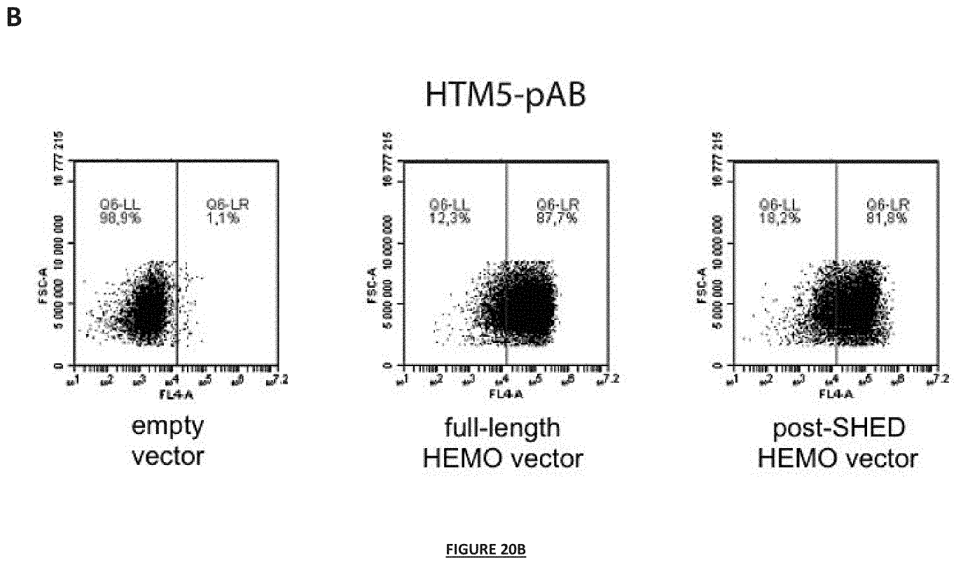

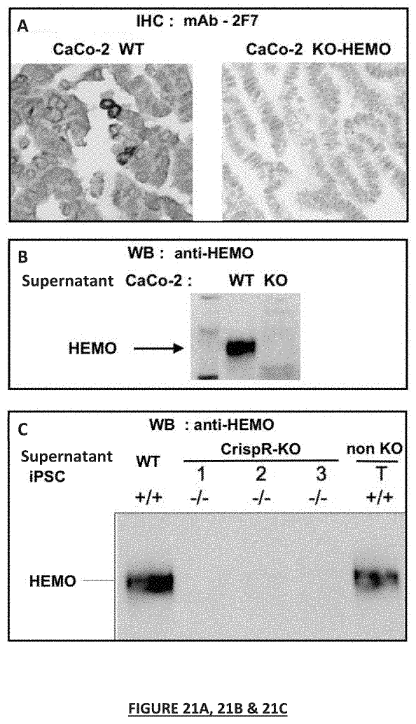

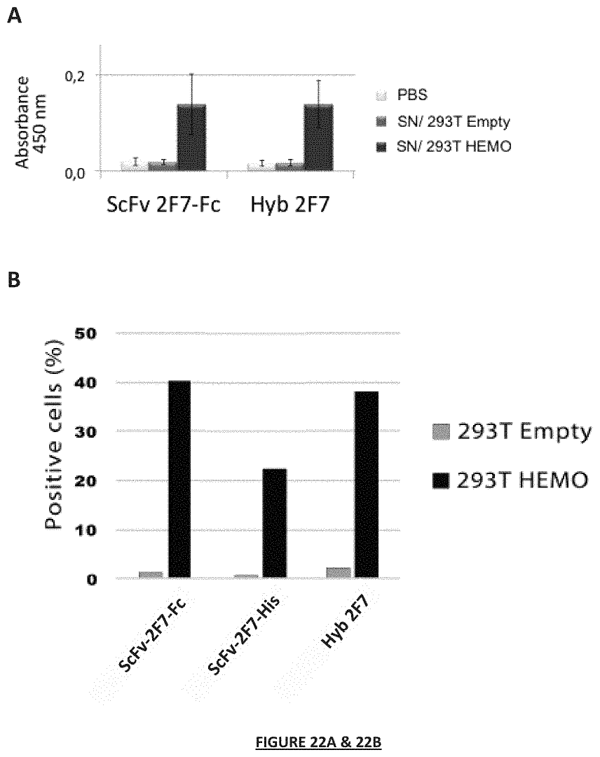

(A, B and C) Amino acid sequence homology between marsupial env-panMars and HEMO proteins from representative simian species and domestic cat. Every amino acid of a marsupial sequence which is found at the same position in a simian or cat sequence is highlight in yellow. Asterisk (*): stop codon. HUM (human), GIB (gibbon), MAC (macaque), MAR (marmoset), CAT (cat), OPO (opossum), WAL (Wallaby) and TAS (Tasmanian Devil). Same color code for the characteristic env domains as in FIG. 1C.

(D) Detection of the HA-tagged Opossum and Wallaby env-panMars proteins. Western Blot of cell lysates (L) and supernatants (S) from 293T cells transfected with the phCMV-empty, phCMV-Opossum-env or phCMV-Wallaby-env expression vectors. Detection with an anti-HA antibody (upper), and with an anti-γ-tubulin antibody (lower).

(E) Structure of the env-panMars gene locus and transcripts for the opossum (upper) and wallaby (lower). Schematic representation of the env-panMars locus, with the env-ORF in orange and the CpG island in green. N represents uncharacterized sequences. Black arrowhead positions the AATAAA polyadenylation signal sequence. Intron-exon structures are from UCSC for the Opossum and were characterized by RACE-PCR experiments for the Wallaby (RNA from the ovaries); nucleotide sequences of the start site (CTTTCTA . . . ) and of the env ORF acceptor splice site are indicated; E2-E3 intron dotted to indicate E3 skipping in part of the Wallaby transcripts, as observed for the HEMO gene.

FIG. 13: Schematic representation of the shedding of the HEMO protein.

“1” is the main cleavage site (C-term end of shed fragment=aa position 432 or 433).

“2” and “3” are secondary cleavage sites (“2”: C-term end of shed fragment is from among aa positions 450-480; “3”: C-term end of shed fragment is from among aa positions 380-420).

FIG. 14: Microarray analysis of HEMO expression within tumor samples.

Box plot representations of normalized values obtained for HEMO gene expression extracted from the GSE2109 dataset (on a logarithmic scale).

FIGS. 15A and 15B: TGCA RNAseq analysis of HEMO expression within tumor samples.

(A) Box plot representations of normalized values obtained for HEMO gene expression (on a FPKM scale). This figure is a copy of an internet TCGA data analyses on a panel of 17 different tumor types, using the complete Mer34-cDNA sequence (about 3000 bp including 5′ and 3′ untranslated sequences).

(B) HEMO expression in RNAseq dataset of a series of TCGA tumors (Head and Neck Squamous Carcinomas, HNSC; Lung AdenoCarninomas, LUAD; Uterine Corpus Endometrial Carcinomas, UCEC), and TCGA controlateral normal tissues (Control).

FIGS. 16A and 16B: HEMO expression in tumor samples from Gustave Roussy.

(A) Western blot analysis on frozen samples of ovarian carcinomas (E=Endometrioid, C=Clear cell) and control (N=Normal ovary, PL=Placenta).

(B) HES staining and HEMO Immunochemistry analysis on FFPE samples of Endometrioid ovarian carcinoma from two patients (I and II), at different magnifications. Normal ovary control is shown in FIG. 9D.

FIG. 17: HEMO expression in tumor samples from Gustave Roussy.

HES staining and HEMO Immunochemistry analysis on FFPE samples of endometrioid uterine carcinomas from two patients (I and II) at different magnifications.

FIG. 18: HEMO expression in tumor samples from Gustave Roussy.

HES staining and HEMO Immunochemistry analysis on FFPE samples of breast carcinomas from two patients (HER2+ and Triple Neg) and normal tissue control, at different magnifications.

FIGS. 19A and 19B: Development of a blood-ELISA assay for detection of circulating HEMO shed protein.

(A) Schematic representation of a sandwich ELISA.

(B) ELISA analysis of circulating HEMO shed protein in woman blood sera.

FIGS. 20A and 20B: Antibodies raised against the C-terminal part of the HEMO-ectodomain

(A) Western blot analysis of transfected 293T (1=full-length HEMO-pHCMV vector, 2=SU-HEMO-pHCMV vector, 3=TM-HEMO-pHCMV vector and 4=post-SHED-HEMO-pHCMV vector) cell lysates with HTM5-polyclonal antibody.

(B) Flow cytometry analysis of transfected 293T cell (left=empty vector, middle=full-length HEMO vector and right=post-SHED HEMO vector) with HTM5-polyclonal antibody. The HTM5 antibody can detect the native form of the full length-HEMO and the C-terminal part of the ectodomain.

FIGS. 21A, 21B and 21C: KO (Knock-Out) cell clones for HEMO by CrispR-Cas9.

(A and B) Immunochemistry and Western blot analysis of WT (Wild Type) and KO-HEMO CaCo-2 cells or supernatants with 2F7 monoclonal antibody (CNCM I-5211). HEMO cannot be detected in CaCo-2 KO HEMO cells, neither by IHC on FFPE cell pellet samples (A), nor in concentrated (20×) supernatant (B).

(C) Western Blot analysis of WT iPSC supernatant, CrispR-KO iPSC supernatants (from cell clones 1, 2 and 3) and non-KO(control CrispR-treated) iPSC supernatant (T) with 2F7 monoclonal antibody (CNCM I-5211). HEMO cannot be detected in supernatant of CrispR-KO iPSC.

FIGS. 22A and 22B: Cloning of the mAB as ScFv fragments.

(A) ELISA analysis of transfected 293T cell (empty vector or full length HEMO vector) supernatant with ScFV-2F7-Fc and 2F7 monoclonal antibody (CNCM I-5211). HEMO is only detected in 293T cell expressing HEMO by both ScFV-2F7-Fc and 2F7 monoclonal antibody (CNCM I-5211).

(B) Flow cytometry analysis of transfected 293T cell (empty vector or full length HEMO vector) with ScFV-2F7-Fc, ScFv-2F7-His and 2F7 monoclonal antibody (CNCM I-5211). HEMO is only detected in 293T cell expressing HEMO by both ScFV fragments (with Fc and His-Tag) and 2F7 monoclonal antibody (CNCM I-5211).

### DETAILED DESCRIPTION

The application relates to the subject-matter as defined in the claims as filed and as herein described.

In the application, unless specified otherwise or unless a context dictates otherwise, all the terms have their ordinary meaning in the relevant field(s).

The application relates to a retroviral Env protein, which is endogenous to the Boreoeutheria mammal clade, more particularly to humans, i.e., to a Human Endogenous RetroVirus (HERV) protein.

Said retroviral Env protein has been named HEMO by the inventors. HEMO stands for Human Endogenous MER34 ORF, but the HEMO protein is not restricted to humans: the HEMO protein is expressed in non-human Boreoeutheria, such as e.g., in non-human primates, as well as in humans (please see FIGS. 10A and 10B).

The RNA transcript of human HEMO has been described in the prior art, e.g., in UNIPROTKB under number Q9H9K5 (MER34_HUMAN) [Name: ERVMER34-1; ORF Name: LP9056]. The sequence of a putative protein has been deduced from said prior art RNA sequence, but the actual occurrence of the protein was hypothetical only.

The inventors provide the demonstration that the protein is actually expressed, and that it is expressed in Boreoeutheria, more particularly in humans. The inventors further describe new functions (characteristics) of the protein.

HEMO is a transmembrane protein: it consists of a signal peptide (which is cleaved off to form the mature protein), an ectodomain, a transmembrane domain and an intracellular domain.

Illustrative sequences of the HEMO proteins comprise:
the human HEMO amino acid sequence of SEQ ID NO: 1 (cf. FIG. 1C, FIG. 1B; cf. sequence HUM in FIGS. 11A-11B and 12A-12C), and
the non-human HEMO proteins of SEQ ID NO: 129-143 (cf. FIGS. 11A-11B and 12A-12C), which are the HEMO protein sequences of chimpanzee (CPZ), gorilla (GOR), orangutan (ORA), gibbon (GIBB), macaque (MAC), baboon (BAB), African Green Monkey (AGM), Colobus (Angolensis palliates) (COL), Langur (LAN), Marmoset (MAR), Rhinopithecus (roxellana) (RHI), Squirrel monkey (SQM), Spider monkey (SPI), Saki monkey (SAK), and cat (CAT), respectively.

In the application, the HEMO reference amino acid sequence, notably for computation of amino acid positions, is the human HEMO protein sequence that includes the N-terminal signal peptide, i.e., the sequence of SEQ ID NO: 1 (563 amino acids).

The sequence alignments shown in FIGS. 11A-11B and 12A-12C enable to identify corresponding positions in non-human Boreoeutheria.

The inventors notably demonstrate that in Boreoeutheria, more particularly in humans, the HEMO protein is highly expressed by placental cells, by stem cells, and by some tumor cells.

The application generally relates to the HEMO protein, the HEMO ectodomain, the HEMO transmembrane domain, and the HEMO intracellular domain, as well as to fragment of these domains.

The inventors further demonstrate that the HEMO protein is cleaved by shedding. Soluble fragments of the HEMO protein can therefore be found in the blood of the Boreoeutheria, more particularly in the circulating blood.

The inventors demonstrate more particularly that the HEMO protein is shed in its ectodomain.

The shedding of the HEMO ectodomain results in the release of soluble fragments, which are N-terminal fragments of the HEMO ectodomain. The C-terminal fragment that results from the cleavage of a (soluble) N-terminal fragment is retained at the cell surface.

The application therefore generally relates to fragments of the HEMO protein, more particularly to fragments of the HEMO ectodomain. The application relates more particularly to:
soluble fragments of the HEMO ectodomain, more particularly to N-terminal soluble fragments of HEMO ectodomain, and to
the fragments of the HEMO ectodomain or of the HEMO protein that result from the shedding of a soluble fragment of HEMO ectodomain, more particularly to the C-terminal fragments of HEMO ectodomain or protein that result from the shedding of a N-terminal soluble fragment of

HEMO ectodomain.

The inventors have identified at least three different cleavage sites in the HEMO ectodomain. The main cleavage site locates in the immunosuppressive domain of the HEMO ectodomain. Other cleavage sites may locate upstream or downstream said immunosuppressive domain (in N- to C-orientation).

The blood, more particularly the circulating blood of a Boreoeutheria, more particularly of a human, may thus comprise one or several of the following three items:
one or several soluble (shed) N-terminal fragments of the HEMO ectodomain, and
one or several cells (or part of cells, e.g. exosomes) that express at their surface a C-terminal fragment of HEMO ectodomain (resulting from the cleavage of one of said soluble (shed) N-terminal fragments).

Placental cells, stem cells and tumor cells of a Boreoeutheria, more particularly of a human, may thus comprise cells (or part of cells, e.g. exosomes), which express at their surface a C-terminal fragment of HEMO ectodomain (which results from the shedding of a N-terminal fragment of HEMO ectodomain). These cells may be part of a cell tissue (e.g., placental cells of a placenta; tumor cells of a tumor tissue; stem cells, which are contained in the bone marrow or in a normal tissue or in a tumor tissue), or may be circulating cells (e.g., circulating placental cells, circulating stem cells or circulating tumor cells).

The application notably relates to:
a polypeptide, which is one of said N-terminal soluble (shed) fragments of HEMO ectodomain, and to
a polypeptide, which is one of said C-terminal fragments of HEMO ectodomain and to a cell (or part of cells, e.g. exosomes), which expresses said C-terminal fragment at its surface.

The application also relates to (sub-)fragments of said polypeptides, more particularly to a (sub-)fragment of said N-terminal soluble (shed) fragments of HEMO ectodomain, wherein said (sub-)fragment is useful for antibody production, more particularly for monoclonal antibody production.

Throughout the application, and unless the context dictates otherwise, a polypeptide [or a polypeptide (sub-)fragment] may be a polypeptide [or a polypeptide (sub-)fragment], which is under soluble form (i.e., non-membranar form).

Throughout the application, and unless the context dictates otherwise, a polypeptide [or a polypeptide (sub-)fragment, a nucleic acid, a nucleic acid vector] may be a polypeptide [or a polypeptide (sub-)fragment, a nucleic acid, a nucleic acid vector, respectively], which is under isolated (or purified) form.

The application also relates to products, which derive from said polypeptides, cell or polypeptide (sub-)fragments. The application relates more particularly to:
a composition, more particularly to a pharmaceutical composition, which comprises at least one polypeptide, cell or polypeptide (sub-)fragment of the application,
an antibody, a monoclonal antibody, a Fab fragment, a Fab′ fragment, a F(ab)2 fragment, a scFv, a sdAb, or the variable domain of a sdAb, which specifically binds to at least one polypeptide, cell or polypeptide (sub-)fragment of the application,
an hybridoma, which produces a monoclonal antibody of the application,
a genetically engineered T cell, more particularly a Chimeric Antigen Receptor T cell (CAR-T cell), wherein the ectodomain of said Chimeric Antigen Receptor (CAR) comprises or is a scFv of the application,
a nucleic acid, more particularly a RNA, which codes for a polypeptide or polypeptide (sub-fragment) of the application,
a nucleic acid vector, which recombinantly comprises at least one nucleic acid of the application,
a host cell, more particularly a genetically engineered cell, which recombinantly comprises at least one nucleic acid or nucleic acid vector of the application,
a nucleic acid probe, which specifically binds to a nucleic acid of the application, or a set of oligonucleotides, which comprises a primer pair (or a primer pair and a probe), wherein said primer pair (or said primer pair and probe) specifically binds(bind) to a nucleic acid of the application,
a kit, which comprises at least one of said products, e.g., at least one antibody, monoclonal antibody, Fab fragment, Fab′ fragment, F(ab)2 fragment, scFv, sdAb, sdAb variable domain or CAR-T cell of the application,
a solid support, such as a synthetic membrane or a nucleic acid chip, onto which is linked at least one of polypeptide, cell or polypeptide (sub-)fragment of the application, or at least one of said products, e.g., a (synthetic) membrane, onto which at least one antibody, monoclonal antibody, Fab fragment, Fab′ fragment, F(ab)2 fragment, scFv, sdAb, sdAb variable domain or CAR-T cell of the application is linked or grafted, or a nucleic acid chip wherein at least one probe of the application is linked or grafted.

The application relates to the uses or applications of at least one polypeptide, polypeptide (sub-)fragment, cell or product of the application, as well as to methods involving at least one polypeptide or product of the application.

### The HEMO Protein

The HEMO protein is expressed in Boreoeutheria, more particularly in humans.

The HEMO protein, more particularly the human HEMO protein, is highly expressed by placental cells, by stem cells, and by some tumor cells.

Throughout the application, mammals or placental mammals notably include a Boreoeutheria, more particularly an Euarchontoglires or a Laurasiatheria, more particularly an Euarchontoglires, more particularly a primate, more particularly a human or a simian.

Throughout the application, a Boreoeutheria include an Euarchontoglires or a Laurasiatheria, more particularly an Euarchontoglires, more particularly a primate, more particularly a human or a simian.

For example, said placental mammal or Boreoeutheria can be a Homo sapiens (a human), a Pan troglodytes (a chimpanzee), a Gorilla (a gorilla), a Pongo (an orangutan), a Hylobates (a gibbon), a Macaca (a macaque), a Papio anubis (a baboon), a Chlorocebus sabeus (an African Green Monkey or AGM), a Colobus angolensis palliatus (a Colobus), a Semnopithecus entellus (a langur), a Rhinopithecus roxellana (a rhinopithecus), a Marmoset (a marmoset), a Saimiri (a squirrel monkey), an Ateles (a spider monkey), or a Pithecia (a saki).

For example, said placental mammal or Boreoeutheria can be a Homo sapiens (a human), a Pan troglodytes (a chimpanzee), a Gorilla (a gorilla), a Pongo (an orangutan), a Hylobates (a gibbon), a Macaca (a macaque), a Papio anubis (a baboon), a Chlorocebus sabeus (an African Green Monkey or AGM), a Colobus angolensis palliatus (a Colobus), a Semnopithecus entellus (a langur), or a Rhinopithecus roxellana (a rhinopithecus).

For example, said placental mammal or Boreoeutheria can be a Homo sapiens (a human), a Pan troglodytes (a chimpanzee), a Gorilla (a gorilla), a Pongo (an orangutan) or a Hylobates (a gibbon). More particularly said placental mammal or Boreoeutheria is a Homo sapiens (a human).

The HEMO protein can be viewed as a retroviral Env protein, which is endogenous to a cell of a Boreoeutheria, more particularly of a human.

Said cell may e.g., be a placental cell (e.g., a trophoblast cell), a stem cell, a tumor cell or a tumor stem cell.

Said trophoblast cell may e.g., be a villous cytotrophoblast, an extravillous cytotrophoblast or a chorionic membrane trophoblast.

Said tumor cell may e.g., be an ovarian cancer, an uterine cancer (more particularly an endometrial cancer, a cervical cancer, a gestational cancer (including placental cancer, e.g., choriocarcinoma)), a breast cancer, a lung cancer, a stomach cancer, a colon cancer, a liver cancer, a kidney cancer, a prostate cancer, an urothelial cancer, a germ cell cancer, a brain cancer, a head and neck cancer, a pancreatic cancer, a thyroid cancer, a thymus cancer, a skin cancer, a bone cancer or a bone marrow cancer. As urothelial cancers encompass carcinomas of the bladder, ureters and renal pelvis, said cancer may also be an urothelial cancer including a bladder cancer, an ureter cancer or a renal pelvis cancer.

The amino acid or nucleic acid sequences are described in Table 8 below, as well as in the Figures and the Examples. A ST25-compliant sequence listing is also provided.

The HEMO reference sequence is the sequence of human HEMO protein of SEQ ID NO: 1 (cf. e.g., FIG. 1C or 11A-11B). The HEMO protein of SEQ ID NO: 1 comprises:
a signal peptide (amino positions 1-26 or 1-24 or 1-25 in SEQ ID NO: 1, i.e., SEQ ID NO: 2, 168 and 169 respectively),
an ectodomain (amino positions 27-489 or 25-489 or 26-489 or 27-488 or 25-488 or 26-488 or 27-491 or 25-491 or 26-491 or 27-486 or 25-486 or 26-486 in SEQ ID NO: 1, i.e., SEQ ID NO: 4 and 172-182 respectively; or amino positions 25-487 or 25-490 or 25-492 or 26-487 or 26-490 or 26-492 or 27-487 or 27-490 or 27-492, i.e., SEQ ID NOs: 910-918 respectively),
a transmembrane domain (e.g. amino positions 490-512 or 489-512 or 492-512 or 487-512 or 490-509 or 489-509 or 492-509 or 487-509 or 490-513 or 489-513 or 492-513 or 487-513 in SEQ ID NO: 1, i.e., SEQ ID NO: 5 and 427-437, respectively), and
an intracellular domain (amino positions 513-563 or 510-563 or 514-563 in SEQ ID NO: 1, i.e., SEQ ID NO: 6, 417 and 418, respectively).

The sequence of HEMO proteins of Boreoeutheria other than humans can be defined as a sequence, which consists of 517-578 amino acids (more particularly of 536, 562, 527, 517 or 578 amino acids, more particularly of 563 or 562 amino acids, more particularly of 563 amino acids), and which is at least 59% identical to SEQ ID NO: 1 (over the entire length of SEQ ID NO: 1).

The expression “at least 59% identical” encompasses at least 60%, at least 61%, at least 62%, at least 63%, at least 64%; at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, more particularly at least 80% identical, more particularly at least 80% identical, more particularly at least 81% identical, more particularly at least 82% identical, more particularly at least 83% identical, more particularly at least 84% identical, more particularly at least 85% identical, more particularly at least 86% identical, more particularly at least 87% identical, more particularly at least 88% identical, more particularly at least 89% identical, more particularly at least 90% identical, more particularly at least 91% identical, more particularly at least 92% identical, more particularly at least 93% identical, more particularly at least 94% identical, more particularly at least 95% identical, more particularly at least 96% identical, more particularly at least 97% identical, more particularly at least 98% identical, more particularly at least 99% identical, to SEQ ID NO: 1 over the entire length of SEQ ID NO: 1. Examples of such non-human HEMO proteins comprise the sequences of SEQ ID NO: 129-143 (cf. FIGS. 11A-11B and 12A-12C), which are the HEMO protein sequences of chimpanzee (CPZ), gorilla (GOR), orangutan (ORA), gibbon (GIB), macaque (MAC), baboon (BAB), African Green Monkey (AGM), Colobus (Angolensis palliates) (COL), Langur (LAN), Marmoset (MAR), Rhinopithecus (roxellana) (RHI), Squirrel monkey (SQM), Spider monkey (SPI), Saki monkey (SAK), and cat (CAT), respectively.

Examples of such non-human HEMO proteins are more particularly the sequences of SEQ ID NO: 129-142 (i.e., from CPZ to SAK), more particularly of SEQ ID NO: 129-138 (i.e., from CPZ to RHI), more particularly of SEQ ID NO: 129-132 (i.e., from CPZ to GIB).

### HEMO Signal Peptide

The signal peptide of the HEMO protein consists of 24, 25 or 26 amino acids, and comprises the sequence of SEQ ID NO: 405.

For example, the signal peptide of the HEMO protein may consist of the sequence of SEQ ID NO: 147 (26 amino acids), of SEQ ID NO: 405 (24 amino acids), or of SEQ ID NO: 406 (25 amino acids).

For example, the signal peptide of the (human) HEMO protein may consist of the sequence of SEQ ID NO: 2 (26 amino acids), of SEQ ID NO: 168 (24 amino acids), or of SEQ ID NO: 169 (25 amino acids).

The mature form of the HEMO protein does not comprise the signal peptide (which has been cleaved off). The human HEMO protein, which matures after cleavage of the signal peptide, may thus be of SEQ ID NO: 3, 170 or 171 (start positions 27, 25 or 26, respectively).

### HEMO Ectodomain

The ectodomain of the HEMO protein comprises, in N-term to C-term orientation,

i. a CWLC amino acid sequence,
ii. an amino acid sequence chosen from among the CTQG sequence, the CTQR sequence, the CIQR sequence, the RTQR sequence and the RTKR sequence,
iii. an optional amino acid sequence of SEQ ID NO: 148 (which features the ImmunoSuppressive Domain [or ISD] of the HEMO protein), and
iv. an amino acid sequence of SEQ ID NO: 149,
which are characteristic of retroviral Env protein.

Please see e.g., the human HEMO sequence shown on FIG. 1C or 4B.

The amino acid sequence of the ectodomain of the HEMO protein may consist of 443-468 amino acids or 443-467 amino acids, more particularly of 460-467 amino acids or of 459-466 amino acids, or of 453-460 amino acids or of 443-450 amino acids, for example of 463 or of 462 or of 456 or of 446 amino acids.

The HEMO ectodomain may start at the first amino acid after the signal peptide (in N- to C-orientation). The HEMO ectodomain ends before the first amino acid of the transmembrane domain (in N- to C-orientation).

The amino acid sequence of ii. is more particularly chosen from among the CTQG sequence, the CTQR sequence and the CIQR sequence. More particularly, the amino acid sequence of ii. is the CTQG sequence.

The optional amino acid sequence of iii., i.e., of SEQ ID NO: 148, features the ImmunoSuppressive Domain [or ISD] of the HEMO protein and is generic for the Boreoeutheria. In the human HEMO protein, said sequence of SEQ ID NO: 148 (ISD) may consist of the sequence of SEQ ID NO: 7.

The amino acid sequence of iv. i.e., of SEQ ID NO: 149, features a CX6CC motif and is generic for the Boreoeutheria. In the human HEMO protein, said sequence of SEQ ID NO: 149 may consist of the sequence of SEQ ID NO: 410.

For example, the HEMO ectodomain may consists of a sequence chosen from among

a/ SEQ ID NO: 4 and 172-182 [human HEMO ectodomain; start positions 27, 25 or 26; stop positions 489, 488, 491 or 486],
b/ SEQ ID NO: 910-918 [human HEMO ectodomain; start positions 25, 26 or 27; stop positions 487, 490 or 492], and
c/ the sequences, which consist of 443-468 or 443-467 amino acids, and which are at least 76% identical to at least one of SEQ ID NO: 4 and 172-182 (over the entire length of said at least one of SEQ ID NO: 4 and 172-182 and 910-918).

The expression at least 76% identical encompasses at least 77%, at least 78%, at least 79%, at least 80%, at least 81%, at least 82%, at least 83%, at least 84%, at least 85%, at least 86%, at least 87%, at least 88% or at least 89% or at least 90% or at least 91% or at least 92% or at least 93% or at least 94% or at least 95% or at least 96% or at least 97% or at least 98% or at least 99% identical.

The sequences of b/ include the HEMO ectodomain of non-human Boreoeutheria. They may e.g., be chosen from among
the sequences of SEQ ID NO: 547-561 and
the sequences, which are fragments of at least one of the sequences of SEQ ID NO: 547-561, and which differ by at most 7 or 8 amino acids in length from said at least one of the sequences of SEQ ID NO: 547-561. The sequences of b/may e.g., be chosen from among the sequences of SEQ ID NO: 442-621.

For example, the (human) HEMO ectodomain may consists of a sequence chosen from among

d/ the sequences of SEQ ID NO: 178, 912 and 547-561, and
e/ the sequences, which are fragments of at least one of the sequences of SEQ ID NO: 172, 911 and 457-471, and which differ by at most 7 amino acids in length from said at least one of the sequences of SEQ ID NO: 178, 912 and 547-561- and

The sequences of d/ and e/ may e.g., be chosen from among the sequences of SEQ ID NO: 4, 172-182 and 442-621.

The sequence, which extends from the first amino acid of the signal peptide to the last amino acid of the ectodomain of the (human) HEMO protein (in N- to C-orientation) may e.g., be a sequence chosen from among the sequences of SEQ ID NO: 438-441 (start position=1; end position=489, 488, 491 or 486).

### HEMO Transmembrane Domain

The transmembrane domain of the HEMO protein extends from the amino acid, which is immediately after the last amino acid of the ectodomain, to the amino acid which immediately precedes the first amino acid of the intracellular domain (in N- to C-orientation).

The amino acid sequence of the transmembrane domain of the HEMO protein may consist of 17 or 18-27 amino acids. It may comprise the sequence of SEQ ID NO: 421

More particularly, the amino acid sequence of said transmembrane domain may consist of 17 or 18-27 amino acids, and may comprise or consist of a sequence chosen from among the sequences of SEQ ID NO: 151, 407-409 and 419-426.

More particularly, the amino acid sequence of the transmembrane domain of the (human) HEMO protein may consist of 17 or 18-27 amino acids, and may comprise the sequence of SEQ ID NO: 432.

More particularly, the amino acid sequence of the transmembrane domain of the (human) HEMO protein may consist of 17 or 18-27 amino acids, and may comprise or consist of a sequence chosen from among the sequences of SEQ ID NO: 5 and 427-437.

More particularly, the amino acid sequence of the transmembrane domain of the (human) HEMO protein consists of a sequence chosen from among the sequences of SEQ ID NO: 5 and 427-437.

### HEMO Intracellular Domain

The intracellular domain of the HEMO protein extends from the amino acid, which is immediately after the last amino acid of the transmembrane, to the last amino acid of the HEMO protein (in N- to C-orientation).

The amino acid sequence of the intracellular domain of the HEMO protein may consist of 20-54 amino acids, e.g., of 30-54, 40-54 or 50-54 amino acids. It may comprise the sequence of SEQ ID NO: 413. More particularly, the amino acid sequence of the intracellular domain of the HEMO protein may consist of a sequence chosen from among the sequences of SEQ ID NO: 411-413.

For example, the amino acid sequence of the intracellular domain of the HEMO protein may consist of 50-54 amino acids and may comprise the sequence of SEQ ID NO: 416. More particularly, the amino acid sequence of the intracellular domain of the HEMO protein may consist of a sequence chosen from among the sequences of SEQ ID NO: 414-416.

For example, the amino acid sequence of the intracellular domain of the (human) HEMO protein may consist of 50-54 amino acids and may comprise the sequence of SEQ ID NO: 418. More particularly, the amino acid sequence of the intracellular domain of the (human) HEMO protein may consist of a sequence chosen from among the sequences of SEQ ID NO: 6, 417 and 418.

### HEMO Shedding

The inventors demonstrate that the HEMO protein is shed in its ectodomain.

The inventors have identified shedding (or cleavage) sites in the HEMO ectodomain, more particularly at least three different shedding sites in the HEMO ectodomain.

These shedding sites can be located in the region, which in the human HEMO protein of SEQ ID NO: 1 extends from amino acid position 380 to amino acid position 480, i.e., in the HEMO polypeptide of SEQ ID NO: 150.

More particularly, at least one of said shedding sites can be located in the region, which in the human HEMO protein of SEQ ID NO: 1 extends from amino acid position 380 to amino acid position 420, or from amino acid position 421 to amino acid position 449, or from amino acid position 450 to amino acid position 480.

More particularly, at least one of said shedding sites can be located in the region, which in the human HEMO protein of SEQ ID NO: 1 extends from amino acid position 421 to amino acid position 449; wherein said shedding sites can be located between amino acid positions 421 and 422, or 422 and 423, or 423 and 424, or 424 and 425, or 425 and 426, or 426 and 427, or 427 and 428, or 428 and 429, or 429 and 430, or 430 and 431, or 431 and 432, or 432 and 433, or 433 and 434, or 434 and 435, or 435 and 436, or 436 and 437, or 437 and 438, or 438 and 439, or 439 and 440, or 440 and 441, or 441 and 442, or 442 and 443, or 443 and 444, or 444 and 445, or 445 and 446, or 446 and 447, or 447 and 448, or 448 and 449.

More particularly, at least one of said shedding sites can be located in the region, which in the human HEMO protein of SEQ ID NO: 1 extends from amino acid position 428 to amino acid position 438, i.e., in the HEMO polypeptide of SEQ ID NO: 623. It is the main shedding site of the HEMO protein, and locates in the immunosuppressive domain of the HEMO ectodomain.

For example, at least one of said shedding sites can be located between amino acid positions 432 and 433, or 433 and 434 (computed by reference to the human HEMO protein of SEQ ID NO: 1; cf. FIG. 11A-11B or 12A-12C to identify the corresponding amino positions in the non-human HEMO proteins).

Other shedding sites may locate upstream or downstream said immunosuppressive domain (in N- to C-orientation).

By reference to the human HEMO protein sequence of SEQ ID NO: 1, a downstream shedding site may locate between two (different) amino acid positions chosen from amino acid positions 450-480 (i.e., it may locate in SEQ ID NO: 624), for example between amino acid positions 472 and 473.

By reference to the human HEMO protein sequence of SEQ ID NO: 1, an upstream shedding site may locate between two (different) amino acid positions chosen from amino acid positions 380-420 (i.e., it may locate in SEQ ID NO: 622), for example between positions 406 and 407.

The shedding of the HEMO ectodomain results in the release of soluble fragments, which are N-terminal fragments of the HEMO ectodomain.

The C-terminal fragment that results from the cleavage of a (soluble) N-terminal fragment is retained at the cell (or part of cells, e.g. exosomes) surface, more particularly at the surface of placental cells (or part of placental cells), of stem cells (or part of stem cells), or of (some) tumor cells (or part of (some) tumor cells).

The inventors notably demonstrate that the shedding of the HEMO protein may be indicative (or may be a marker) of pluripotency and/or of a tumorigenic nature.

### Soluble N-Terminal Fragments of HEMO Ectodomain (Produced by Shedding of the HEMO Protein)

The expression “polypeptide in soluble form” (or similar expression) is intended in accordance with its ordinary meaning in the art. The expression generally refers to an acellular or cell-free polypeptide, i.e., which is not contained in or linked to a cell. The expression refers more particularly to a polypeptide which is not membranar, not transmembranar, and not cytosolic.

The application thus relates to a polypeptide, the amino acid sequence of which is the sequence of a fragment, more particularly the sequence of a N-terminal fragment, of the ectodomain of a retroviral Env protein, wherein said retroviral Env protein is the HEMO protein as above-defined.

Said HEMO protein can e.g., be defined as a retroviral Env protein, which is endogenous to a Boreoeutheria, wherein the amino acid sequence of the HEMO protein may e.g., be

a. the sequence of SEQ ID NO: 1, or the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1, or
b. a sequence, which consists of 517-578 amino acids, and which is at least 78% identical to SEQ ID NO: 1 over the entire length of SEQ ID NO: 1.

The amino acid sequence of the ectodomain of said retroviral Env protein is as above-defined, e.g., it may consist of a sequence of 443-467 amino acids, which may comprise, in N-term to C-term orientation,

i. a CWLC amino acid sequence,
ii. an amino acid sequence chosen from among the CTQG sequence, the CTQR sequence, the CIQR sequence, the RTQR sequence and the RTKR sequence,
iii. an optional amino acid sequence of SEQ ID NO: 148 (which features the ISD of the HEMO protein), and
iv. an amino acid sequence of SEQ ID NO: 149.

The sequence of the fragment of said ectodomain consists of a number of amino acids lower than said ectodomain, said lower number being more particularly chosen from among 344-457, or 352-457, or 354-456, or 374-446, more particularly from among 344-373, or 354-373, or 406-409, or 374-396, or 424-446, or 447-456, or 447-457.

The sequence of the fragment of said ectodomain may comprise said sequence of i. and said sequence of ii.

The application uses the HEMO protein sequences of human as a reference and comprises the HEMO protein sequences of chimpanzee (CPZ), gorilla (GOR), orangutan (ORA), gibbon (GIB), macaque (MAC), baboon (BAB), African Green Monkey (AGM), Colobus (Angolensis palliates) (COL), Langur (LAN), Marmoset (MAR), Rhinopithecus (roxellana) (RHI), Squirrel monkey (SQM), Spider monkey (SPI), Saki monkey (SAK), and cat (CAT), wherein corresponding the different motifs and positions of the ectodomain herein defined are as follows:

TABLE 1
Equivalent positions and motifs on Boreoeutheria HEMO proteins as compared to Human HEMO protein.

| | | | | | | | | | Ectodomain | |
|---|---|---|---|---|---|---|---|---|---|---|
| HEMO protein | | | Motifs (from aa to aa) | | | | Shedding | | | End |
| SEQ ID NO | Species | size (aa) | CWLC | Furin motif | ISU | CXX6CC | Start | End | Start | (from aa to aa) |
| 1 | Human | 563 | 44-47 | 352-355 | 420-436 | 437-445 | 380 | 480 | 25, 26 or 27 | 486-492 |
| 129 | CPZ | 563 | 44-47 | 352-355 | 420-436 | 437-445 | 380 | 480 | 25, 26 or 27 | 486-492 |
| 130 | GOR | 563 | 44-47 | 352-355 | 420-436 | 437-445 | 380 | 480 | 25, 26 or 27 | 486-492 |
| 131 | OO | 563 | 44-47 | 352-355 | 420-436 | 437-445 | 380 | 480 | 25, 26 or 27 | 486-492 |
| 132 | GIB | 563 | 44-47 | 352-355 | 420-436 | 437-445 | 380 | 480 | 25, 26 or 27 | 486-492 |
| 133 | MAC | 563 | 44-47 | 352-355 | 420-436 | 437-445 | 380 | 480 | 25, 26 or 27 | 486-492 |
| 134 | BAB | 563 | 44-47 | 352-355 | 420-436 | 437-445 | 380 | 480 | 25, 26 or 27 | 486-492 |
| 135 | AGM | 562 | 44-47 | 351-354 | 419-435 | 436-444 | 379 | 479 | 25, 26 or 27 | 485-491 |
| 136 | COL | 563 | 44-47 | 352-355 | 420-436 | 437-445 | 380 | 480 | 25, 26 or 27 | 486-492 |
| 137 | LAN | 563 | 44-47 | 352-355 | 420-436 | 437-445 | 380 | 480 | 25, 26 or 27 | 486-492 |
| 138 | RHI | 562 | 44-47 | 352-355 | 419-435 | 436-444 | 380 | 479 | 25, 26 or 27 | 485-491 |
| 139 | MAR | 527 | 44-47 | 352-355 | 420-436 | 437-445 | 380 | 476 | 25, 26 or 27 | 479-485 |
| 140 | SQM | 527 | 44-47 | 352-355 | 420-436 | 437-445 | 380 | 476 | 25, 26 or 27 | 479-485 |
| 141 | SPI | 517 | 44-47 | 342-345 | 410-426 | 427-435 | 370 | 466 | 25, 26 or 27 | 469-475 |
| 142 | SAK | 527 | 44-47 | 352-355 | 420-436 | 437-445 | 380 | 476 | 25, 26 or 27 | 479-485 |
| 143 | CAT | 578 | 44-47 | 352-355 | 418-433 | 434-442 | 378 | 479 | 24, 25, 26 or 27 | 485-491 |

The general aspect of the invention thus relates to a polypeptide consisting of a N-terminal fragment of the ectodomain of an endogenous Boreoeutheria retroviral Env protein,
a. wherein said retroviral Env protein is
   the amino acid sequence of SEQ ID NO: 1; or
   the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1; or
   a sequence chosen from among the sequences of SEQ ID NOs: 129-143,
b. wherein said ectodomain, without its signal peptide or fragment thereof, of said retroviral Env protein consists of:
   443-468 or 443-467 amino acids of said sequences which more particularly comprise in N-term to C-term orientation
      i. a CWLC amino acid sequence,
      ii. an amino acid sequence chosen from among the CTQG sequence, the CTQR sequence, the CIQR sequence, the RTQR sequence and the RTKR sequence, and
      iv. an amino acid sequence of SEQ ID NO: 149;
wherein the ectodomain of said retroviral Env protein consists more particularly of a sequence chosen from among the sequences of SEQ ID NOs: 178, 912 and 547-561, and the sequences, which are the fragments of at least one of the sequences of SEQ ID NOs: 172, 911 and 457-471, and which differ by at most 7 or 8 amino acids in length of at least one of the sequences of SEQ ID NOs: 178, 912 and 547-561,
wherein the sequence of said N-terminal fragment:
   consists of a number of amino acids lower than said ectodomain, said lower number being chosen from among 344-457 or 374-446,
   starts at the N-terminal extremity of said ectodomain; and
   comprises said sequence of b.i. and said sequence of b.ii.

According to a particular embodiment, the invention relates to a polypeptide consisting of a N-terminal fragment of the ectodomain of an endogenous Boreoeutheria (e.g. Human, CPZ, GOR, ORA, GIB, MAC, BAB, AGM, COL, LAN, MAR, RHI, SQM, SPI, SAK) retroviral Env protein as defined above,
a. wherein said retroviral Env protein is
   the amino acid sequence of SEQ ID NO: 1; or
   the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1; or
   a sequence chosen from among the sequences of SEQ ID NOs: 129-142,
b. wherein said ectodomain, without its signal peptide or fragment thereof, of said retroviral Env protein consists of:
   443-468 or 443-467 amino acids of said sequences which more particularly comprise in N-term to C-term orientation
      i. a CWLC amino acid sequence,
      ii. an amino acid sequence chosen from among the CTQG sequence, the CTQR sequence, the CIQR sequence and the RTKR sequence,
      iii. an amino acid sequence of SEQ ID NO: 148, and
      iv. an amino acid sequence of SEQ ID NO: 149;
wherein the ectodomain of said retroviral Env protein consists more particularly of a sequence chosen from among the sequences of SEQ ID NOs: 178, 912 and 547-560, and the sequences, which are the fragments of at least one of the sequences of SEQ ID NOs: 172, 911 and 457-470, and which differ by at most 7 or 8 amino acids in length of at least one of the sequences of SEQ ID NOs: 178, 912 and 547-560,
wherein the sequence of said N-terminal fragment:
   consists of a number of amino acids lower than said ectodomain, said lower number being chosen from among 344-457 or 374-446,
   starts at the N-terminal extremity of said ectodomain; and
   comprises said sequence of b.i. and said sequence of b.ii.

More particularly, the above mention peptides are N-terminal fragments of the ectodomain of an endogenous Boreoeutheria retroviral Env protein as defined above, wherein the sequence of said N-terminal fragments consist of a number of amino acids lower than said ectodomain, said lower number being chosen from among 354-456 or 374-446.

According to another particular embodiment, the invention relates to a polypeptide consisting of a N-terminal fragment of the ectodomain of an endogenous Boreoeutheria (e.g. Human, CPZ, GOR, ORA, GIB, MAC, BAB, AGM, COL, LAN, MAR, RHI, SQM, SPI, SAK) retroviral Env protein as defined above, wherein
a. the C-terminal extremity of said N-terminal fragment is located from amino acid at position 380 to amino acid at position 480 of the sequences SEQ ID NOs: 1, 129-134 and 136-137 and before the N-terminal extremity of the transmembrane domain of the sequences SEQ ID NOs: 1, 129-134 and 136-137 at a location of 6 to 112 amino acids upstream the N-terminal extremity of the transmembrane domain of the sequences SEQ ID NOs: 1, 129-134 and 136-137; or
b. the C-terminal extremity of said N-terminal fragment is located from amino acid at position 379 to amino acid at position 479 of the sequence SEQ ID NO: 135 and before the N-terminal extremity of the transmembrane domain of the sequence SEQ ID NO: 135 at a location of 6 to 112 amino acids upstream the N-terminal extremity of the transmembrane domain of the sequence SEQ ID NO: 135; or
c. the C-terminal extremity of said N-terminal fragment is located from amino acid at position 380 to amino acid at position 479 of the sequence SEQ ID NO: 138 and before the N-terminal extremity of the transmembrane domain of the sequence SEQ ID NO: 138 at a location of 6 to 111 amino acids upstream the N-terminal extremity of the transmembrane domain of the sequence SEQ ID NO: 138; or
d. the C-terminal extremity of said N-terminal fragment is located from amino acid at position 380 to amino acid at position 476 of the sequences SEQ ID NOs: 139-140 and 142 and before the N-terminal extremity of the transmembrane domain of the sequences SEQ ID NOs: 139-140 and 142 at a location of 3 to 105 amino acids upstream the N-terminal extremity of the transmembrane domain of the sequences SEQ ID NOs: 139-140 and 142; or
e. the C-terminal extremity of said N-terminal fragment is located from amino acid at position 370 to amino acid at position 466 of the sequence SEQ ID NO: 141 and before the N-terminal extremity of the transmembrane domain of the sequence SEQ ID NO: 141 at a location of 3 to 105 amino acids upstream the N-terminal extremity of the transmembrane domain of the sequence SEQ ID NO: 141.

The expression “before the N-terminal extremity of the transmembrane domain of the sequences SEQ ID NOs: 1, 129-134 and 136-137 at a location of 6 to 112 amino acids upstream the N-terminal extremity of the transmembrane domain of the sequences SEQ ID NOs: 1, 129-134 and 136-137” corresponds to the fact that, e.g.,

if the human HEMO ectodomain corresponds to positions 27-492 of the sequence SEQ ID NO:1, the N-terminal extremity of the transmembrane domain is the amino acid 493 of the sequence SEQ ID NO:1;
in consequence, if the N-terminal fragment of the human HEMO ectodomain has a size of 354 amino acids, said N-terminal fragment of the human HEMO ectodomain goes from amino acid 27 to amino acid 380 of the sequence SEQ ID NO:1, the amino acid at position 380 being at a location of 112 amino acids upstream the amino acid at position 493 which is the N-terminal extremity of the transmembrane domain of the sequence SEQ ID NO:1;
and if the human HEMO ectodomain corresponds to positions 25-486 of the sequence SEQ ID NO:1, the N-terminal extremity of the transmembrane domain is the amino acid 487 of the sequence SEQ ID NO:1;
in consequence, if the N-terminal fragment of the human HEMO ectodomain has a size of 456 amino acids, said N-terminal fragment of the human HEMO ectodomain goes from amino acid 25 to amino acid 480 of the sequence SEQ ID NO:1, the amino acid at position 480 being at a location of 6 amino acids upstream the amino acid at position 487 which is the N-terminal extremity of the transmembrane domain of the sequence SEQ ID NO:1

According to another particular embodiment, the invention relates to a polypeptide consisting of a N-terminal fragment of the ectodomain of an endogenous human retroviral Env protein as defined above,
a. wherein said human retroviral Env protein is
   the amino acid sequence of SEQ ID NO: 1; or
   the amino acid sequence which is at least 80% identical to SEQ ID NO: 1, more particularly the amino acid sequence which is at least 81%, 82%, 83%, 84%, 85%, 86%, 87%, 88%, 89%, 90%, 91%, 92%, 93%, 94%, 95%, 96%, 97%, 98% or 99% identical to SEQ ID NO: 1,
b. wherein said ectodomain, without its signal peptide or fragment thereof, of said retroviral Env protein consists of:
   443-468 or 443-467 amino acids of said sequences which more particularly comprise in N-term to C-term orientation
      i. a CWLC amino acid sequence,
      ii. an amino acid sequence chosen from among the CTQG sequence, the CTQR sequence, the CIQR sequence and the RTKR sequence,
      iii. an amino acid sequence of SEQ ID NO: 148, and
      iv. an amino acid sequence of SEQ ID NO: 149;
wherein the ectodomain of said human retroviral Env protein consists more particularly of:
   a sequence chosen from among the sequences of SEQ ID NOs: 178 and 912 and the sequences, which are the fragments of at least one of the sequences of SEQ ID NOs: 172 and 911, and which differ by at most 7 or 8 amino acids in length of at least one of the sequences of SEQ ID NO: 178 and 912,
wherein the sequence of said N-terminal fragment:
   consists of a number of amino acids lower than said ectodomain, said lower number being chosen from among 354-456 or 374-446,
   starts at the N-terminal extremity of said ectodomain;
   comprises said sequence of b.i. and said sequence of b.ii.; and
   the C-terminal extremity of said N-terminal fragment is located from amino acid at position 380 to amino acid at position 480 of the sequence SEQ ID NO: 1 and before the N-terminal extremity of the transmembrane domain of the sequence SEQ ID NO: 1 at a location of 6 to 112 amino acids upstream the N-terminal extremity of the transmembrane domain of the sequence SEQ ID NO: 1.

The sequence of said (soluble) ectodomain fragment may not comprise the full-length sequence of iii., but may comprise a fragment of said sequence of iii. It is notably the case when the shedding site is the main shedding site as described above. For example, the sequence of the (soluble) ectodomain fragment may comprise or consist of a sequence chosen from among the sequences of SEQ ID NO: 9-10, 183-184 and 185-186.

The sequence of said (soluble) ectodomain fragment may not comprise the sequence of iii., and may not comprise any fragment of the sequence of iii. It is notably the case when the shedding site is a secondary (upstream) shedding site as described above. For example, the sequence of said (soluble) ectodomain fragment may comprise or consist of a sequence chosen from among the sequences of SEQ ID NO: 13-33, 670-689, 195-215, 690-709, 216-236 and 710-729.

The sequence of said (soluble) ectodomain fragment may comprise the sequence of iii. It is notably the case when the shedding site is a secondary (downstream) shedding site as described above. For example, the sequence of said (soluble) ectodomain fragment may comprise or consist of a sequence chosen from among the sequences of SEQ ID NO: 55-75, 830-839, 300-320, 840-849, 321-341 and 850-859.

More particularly, the invention relates to a polypeptide as defined above,
a. wherein the sequence of the N-terminal fragment of said ectodomain does not comprise the full-length sequence of b.iii., but comprises a fragment of said sequence of b.iii.;
b. more particularly wherein the sequence of the N-terminal fragment of said ectodomain comprises or consists of a sequence chosen from among the sequences of SEQ ID NOs: 9-10, 183-184 and 185-186.

More particularly, the invention relates to polypeptide as defined above,
a. wherein the sequence of the N-terminal fragment of said ectodomain does not comprise the sequence of claim 2.b.iii., and does not comprise any fragment of the sequence of b.iii.,
   more particularly wherein the sequence of the N-terminal fragment of said ectodomain comprises or consists of a sequence chosen from among the sequences of SEQ ID NO: 13-33, 670-689, 195-215, 690-709, 216-236 and 710-729;
or
b. wherein the sequence of the N-terminal fragment of said ectodomain comprises the sequence of b.iii.,
   more particularly wherein the sequence of the N-terminal fragment of said ectodomain comprises or consists of a sequence chosen from among the sequences of SEQ ID NOs: 55-75, 830-839, 300-320, 840-849, 321-341 and 850-859.

More particularly, the invention relates to polypeptide as defined above,
a. wherein the sequence of the N-terminal fragment of said ectodomain starts at the N-terminal extremity of said ectodomain, said N-terminal extremity of said ectodomain corresponding to amino acid at position 25 of the sequence SEQ ID NO: 1, and
   wherein the C-terminal extremity of said N-terminal fragment corresponds to amino acid at position 421, 422, 423, 424, 425, 426, 427, 428, 429, 430, 431, 432, 433, 434, 435, 436, 437, 438, 439, 440, 441, 442, 443, 444, 445, 445, 446, 447, 448 or 449 of the sequence SEQ ID NO: 1; or
b. wherein the sequence of the N-terminal fragment of said ectodomain starts at the N-terminal extremity of said ectodomain, said N-terminal extremity of said ectodomain corresponding to amino acid at position 26 of the sequence SEQ ID NO: 1, and
   wherein the C-terminal extremity of said N-terminal fragment corresponds to amino acid at position 421, 422, 423, 424, 425, 426, 427, 428, 429, 430, 431, 432, 433, 434, 435, 436, 437, 438, 439, 440, 441, 442, 443, 444, 445, 445, 446, 447, 448 or 449 of the sequence SEQ ID NO: 1; or
c. wherein the sequence of the N-terminal fragment of said ectodomain starts at the N-terminal extremity of said ectodomain, said N-terminal extremity of said ectodomain corresponding to amino acid at position 27 of the sequence SEQ ID NO: 1, and
   wherein the C-terminal extremity of said N-terminal fragment corresponds to amino acid at position 421, 422, 423, 424, 425, 426, 427, 428, 429, 430, 431, 432, 433, 434, 435, 436, 437, 438, 439, 440, 441, 442, 443, 444, 445, 445, 446, 447, 448 or 449 of the sequence SEQ ID NO: 1.

More particularly, the invention relates to polypeptide as defined above, wherein the sequence of the N-terminal fragment of said ectodomain comprises or consists of a sequence chosen from among the sequences of SEQ ID NOs: 9-10, 184-186 and 991-1071.

According to a particular embodiment, the invention relates to a polypeptide consisting of a N-terminal fragment of the ectodomain of an endogenous cat retroviral Env protein as defined above,
a. wherein said retroviral Env protein is
   the amino acid sequence of SEQ ID NO: 143; or
   the amino acid sequence which is at least 80% identical to SEQ ID NO: 143, more particularly the amino acid sequence which is at least 81%, 82%, 83%, 84%, 85%, 86%, 87%, 88%, 89%, 90%, 91%, 92%, 93%, 94%, 95%, 96%, 97%, 98% or 99% identical to SEQ ID NO: 143,
b. wherein said ectodomain, without its signal peptide or fragment thereof, of said retroviral Env protein consists of 443-468 or 443-467 amino acids of said sequences which more particularly comprise in N-term to C-term orientation
   i. a CWLC amino acid sequence,
   ii. a RTQR sequence, and
   iv. an amino acid sequence of SEQ ID NO: 149;
wherein the ectodomain of said retroviral Env protein consists more particularly of the sequences of SEQ ID NOs: 561 and 951, and the sequences, which are the fragments of the sequences of SEQ ID NOs: 471 and 949, and which differ by at most 7 or 8 amino acids in length of at least one of the sequences of SEQ ID NOs: 561 and 951,
wherein the sequence of said N-terminal fragment:
   consists of a number of amino acids lower than said ectodomain, said lower number being chosen from among 352-457 or 374-446;
   starts at the N-terminal extremity of said ectodomain; and
   comprises said sequence of b.i. and said sequence of b.ii.

More particularly, the invention relates to polypeptide as defined above having a percentage of identity of at least 90%, 91%, 92%, 93%, 94%, 95%, 96%, 97%, 98% or 99% with said polypeptide herein defined.

In particular, the above mentioned peptides of the invention are under a lyophilized form or a concentrated form (e.g. from 0.001 to 10 000 nM, or from 0.01 to 10 000 nM, or from 0.001 to 1 000 nM, or from 0.01 to 1 000 nM, or from 0.1 to 1 000 nM, or from 0.1 to 100 nM, or from 0.1 to 10 nM, or from 10 to 100 nM, or from 10 to 1 000 nM, or from 100 to 1 000 nM, or from 1 000 to 10 000 nM or from 5 000 to 10 000 nM) in any physiologically and acceptable carrier.

Said (soluble) ectodomain fragment may comprise the first amino acid of the HEMO ectodomain (in N-term to C-term orientation).

Said (soluble) ectodomain fragment may be herein referred to as the soluble polypeptide, or the N-terminal fragment, or the soluble N-terminal ectodomain fragment.

(Sub-)Fragments of the soluble N-Terminal Ectodomain Fragments, which May be Useful e.g., for Antibody Production

The application also relates to (sub-)fragments of the above-described (soluble) ectodomain fragments. These (sub-)fragments notably encompass (sub-)fragments, which are useful for antibody production, more particularly for monoclonal antibody production (cf. example 2 below).

The application thus relates to a (sub-)fragment of said (soluble) N-terminal fragments of HEMO ectodomain (soluble N-terminal fragments produced by shedding of the HEMO protein), wherein said (sub-)fragment comprises:
at least 10 amino acids, more particularly at least 50 amino acids, 100 amino acids, more particularly at least 150 amino acids, more particularly at least 160 amino acids, more particularly at least 164 amino acids; and/or
less than 400 amino acids, more particularly less than 300 amino acids, more particularly less than 250 amino acids, more particularly less than 200 amino acids.

For example, said (sub-)fragment may comprise at least 100 amino acids and less than 200 amino acids, for example 164-199 amino acids, for example 164 amino acids.

For example, said (sub-)fragment may comprise the sequence of SEQ ID NO: 8.

Said (sub-)fragment advantageously comprise at least one antigen or epitope. Said (sub-)fragment may be immunogenic e.g., when administered to a mouse, for example by systemic administration.

C-Terminal Fragments of HEMO Ectodomain (Resulting from the Shedding of One of Said N-Terminal Ectodomain Fragments), and Cells, which Express Said C-Terminal Fragment at their Surface

The application also relates to the (C-terminal) fragments of HEMO ectodomain or of HEMO protein, which result from the shedding of one of said soluble N-terminal ectodomain fragments.

The application also relates to cells (or part of cells, e.g. exosomes), which express (or onto which has been retained) such a (C-terminal) fragment of HEMO ectodomain or of HEMO protein.

The application thus relates to a polypeptide, the amino acid sequence of which is the sequence of a fragment of the HEMO protein (as defined above), wherein said fragment comprises a C-terminal fragment of the ectodomain of the HEMO protein,

wherein said fragment of retroviral Env protein does not comprise the full-length amino acid sequence of said ectodomain, more particularly wherein said C-terminal fragment comprises the C-terminal end of said ectodomain without comprising the N-terminal end of said ectodomain, and
wherein said C-terminal fragment of ectodomain is the C-terminal fragment, which remains after shedding (or cleavage) of one of said soluble N-terminal ectodomain fragments.

The sequence of said polypeptide may comprise (or the sequence of C-terminal fragment of ectodomain may consist of):

a sequence chosen from among SEQ ID NO: 11-12, 189-190, 191-192 and 193-194; or
a sequence chosen from among SEQ ID NO: 34-54, 730-749, 237-257, 750-769, 258-278, 770-789, 279-299 and 790-809; or
a sequence chosen from among SEQ ID NO: 76-96, 860-869, 342-362, 870-, 879, 363-383, 880-889, 384-404 and 890-899.

For example, the sequence of said polypeptide may e.g., be chosen from among the sequences of SEQ ID NO: 625-626, 627-647, 810-829, 648-668 and 900-909.

Said polypeptide may be herein referred to as the C-terminal protein fragment.

The application also relates to an (isolated) cell, more particularly a naturally-occurring or genetically engineered cell, which expresses said C-terminal protein fragment, wherein a portion of the C-terminal protein fragment is expressed at the surface of said cell, and wherein said surface-expressed portion comprises the C-terminal fragment of ectodomain which is comprised in said C-terminal protein fragment.

The application also relates to a polypeptide, the amino acid sequence of which is the sequence of a C-terminal fragment of the ectodomain of the HEMO protein,

wherein said HEMO protein and said ectodomain are as herein defined, and
wherein said C-terminal fragment is the C-terminal fragment of said ectodomain, which remains after shedding (or cleavage) of one of said soluble N-terminal ectodomain fragments from said ectodomain.

Said polypeptide may consist of:

a sequence chosen from among SEQ ID NO: 11-12, 189-190, 191-192 and 193-194; or
a sequence chosen from among SEQ ID NO: 34-54, 730-749, 237-257, 750-769, 258-278, 770-789, 279-299 and 790-809; or
a sequence chosen from among SEQ ID NO: 76-96, 860-869, 342-362, 870-, 879, 363-383, 880-889, 384-404 and 890-899.

Said polypeptide may be referred to as C-terminal ectodomain fragment.

The application also relates to a polypeptide, the sequence of which, in N-term to C-term orientation, starts with the sequence of a C-terminal ectodomain fragment (as described above), and wherein the C-terminal end of sequence of C-terminal ectodomain fragment is (directly) linked to a transmembrane domain of the HEMO protein, or to a transmembrane domain and to an intracellular domain of the HEMO protein, wherein said HEMO protein is as herein defined.

The application also relates to an (isolated) cell, more particularly a naturally-occurring or genetically engineered cell, which expresses said C-terminal ectodomain fragment, wherein said C-terminal ectodomain fragment is expressed at the surface of said cell.

Said cell may be a naturally-occurring (but isolated) cell, or a genetically engineered cell.

Said cell may be a Boreoeutheria cell, more particularly a human cell.

Said cell may be a placental cell (e.g., a trophoblast), a stem cell, a tumor cell, or a tumor stem cell. Said placental cell may e.g., be a trophoblast cell, more particularly a villous cytotrophoblast, an extravillous cytotrophoblast or a chorionic membrane trophoblast.

Said tumor cell may e.g., be an ovarian tumor, an uterine tumor (more particularly an endometrial tumor, a cervical tumor, a gestational tumor (including placental tumor, e.g., choriocarcinoma)), a breast tumor, a lung tumor, a stomach tumor, a colon tumor, a liver tumor, a kidney tumor, a prostate tumor, an urothelial tumor, a germ cell tumor, a brain tumor, a head and neck tumor, a pancreatic tumor, a thyroid tumor, a thymus tumor, a skin tumor, a bone tumor or a bone marrow tumor. As urothelial tumors encompass carcinomas of the bladder, ureters and renal pelvis, said tumor may also be an urothelial tumor including a bladder tumor, an ureter tumor or a renal pelvis tumor.

According to a particular embodiment, the invention relates to a polypeptide consisting of a fragment of the ectodomain of an endogenous Boreoeutheria retroviral Env protein, wherein said fragment comprises a C-terminal fragment of the ectodomain of said retroviral Env protein,
a. wherein said retroviral Env protein and said ectodomain are as herein defined,
b. wherein said C-terminal fragment comprises the C-terminal end of said ectodomain without comprising the N-terminal end of said ectodomain,
c. wherein said C-terminal fragment of ectodomain is the C-terminal fragment, which remains after cleavage of the previously defined polypeptide from said ectodomain, more particularly wherein the sequence of said C-terminal fragment of ectodomain consists of a sequence chosen from among SEQ ID NOs: 11-12 and 189-194, or from among SEQ ID NOs: 34-54, 237-299 and 730-809, or from among SEQ ID NOs: 76-96, 342-404 and 860-899.

More particularly, the invention relates to polypeptide as defined above having a percentage of identity of at least 90%, 91%, 92%, 93%, 94%, 95%, 96%, 97%, 98% or 99% with said polypeptide herein defined.

(Sub-)Fragments of the C-Terminal Fragments of HEMO Ectodomain (Resulting from the Shedding of One of Said N-Terminal Ectodomain Fragments), which May be Useful e.g., for Antibody Production

The application also relates to (sub-)fragments of the above-described C-terminal fragments of HEMO ectodomain (which are retained on the cell surface after shedding of the soluble N-terminal fragments). These (sub-)fragments notably encompass (sub-)fragments, which are useful for antibody production, more particularly for monoclonal antibody production (cf. example 3 below).

The application thus relates to a (sub-)fragment of said C-terminal fragments of HEMO ectodomain, wherein said (sub-)fragment comprises:
at least 10 amino acids, more particularly at least 15, 16 or 17 amino acids; and/or
less than 200 amino acids, more particularly less than 30 amino acids.

Said (sub-)fragment advantageously comprise at least one antigen or epitope. Said (sub-)fragment may be immunogenic e.g., when administered to a mouse, for example by systemic administration.

Products or Uses, which Comprise or Derive from a Polypeptide, Polypeptide (Sub-)Fragment or Cell of the Application

The application also relates to products or uses, which comprise, involves or directly derive from said soluble N-terminal ectodomain fragments, said (sub-)fragments of (soluble) ectodomain fragments, said C-terminal protein fragments, said C-terminal ectodomain fragments and said cells.

More particularly, the application relates to said soluble N-terminal ectodomain fragments, said C-terminal protein fragments, said C-terminal ectodomain fragments and said cells for use in diagnosis, e.g., in the diagnostic of a tumor, in the diagnostic of a placentation defect, or in the diagnostic of a defect in the protection of a fetus against microbial (more particularly viral) infection in a pregnant Boreoeutheria; or therapy, e.g., in the treatment of a tumor, in the treatment of a placentation defect, or in the treatment of a defect in the protection of a fetus against microbial (more particularly viral) infection in a pregnant Boreoeutheria.

More particularly, the application relates to said (sub-)fragments of (soluble) ectodomain fragments, for use in the production of antibodies, more particularly of monoclonal antibodies by immunization in a non-human mammal.

The application also relates to any composition, more particularly any pharmaceutical composition, which comprises at least one polypeptide or cell of the application, more particularly at least one of said soluble N-terminal ectodomain fragments, said C-terminal protein fragments, said C-terminal ectodomain fragments and said cells. Said composition (or pharmaceutical composition) may optionally further comprise at least one (pharmaceutically acceptable) vehicle, and/or blood or tissue cells from a Boreoeutheria and/or at least one immune adjuvant, and/or at least one buffer.

More particularly, the application relates to a composition, which comprises at least one of said soluble N-terminal ectodomain fragments and/or one of said cells, and which optionally further comprises blood of a Boreoeutheria.

More particularly, the application relates to a composition or pharmaceutical composition or drug, which comprises at least one of said soluble N-terminal ectodomain fragments, and which optionally further comprises at least one pharmaceutically acceptable vehicle. Such a pharmaceutical composition or drug might be useful e.g., in the treatment of a defect in placentation, or in the treatment of a defect in the protection of a fetus against microbial (more particularly viral) infection in a pregnant Boreoeutheria.

More particularly, the application relates to a composition or pharmaceutical composition, which comprises at least one of said (sub-)fragments of (soluble) ectodomain fragments, and which optionally further comprises at least one vaccine adjuvant. This composition may be useful for administration to a non-human mammal to produce antibodies, more particularly monoclonal antibodies which binds to, more particularly which specifically binds to, a polypeptide of the application.

More particularly, the application relates to a composition or pharmaceutical composition, which comprises at least one of said C-terminal protein fragments and said C-terminal ectodomain fragments, and which may optionally further comprise at least one buffer.

More particularly, the application relates to a composition or pharmaceutical composition, which comprises at least one of said cells (which expresses at least one of said C-terminal protein fragments and said C-terminal ectodomain fragments), and which may optionally further comprise blood or cell tissue from a Boreoeutheria.

More particularly, the application relates to a composition or pharmaceutical composition or drug, which comprises at least one of said cells or part of said cells, e.g. exosomes, (which expresses at least one of said C-terminal protein fragments and said C-terminal ectodomain fragments), and which optionally further comprises at least one pharmaceutically acceptable vehicle. Such a pharmaceutical composition or drug might be useful e.g., in the treatment of a defect in placentation.

The expression “cell” includes cell and part of a cell such as exosome, more particularly the expression “cell” includes cell and exosome.

The application also relates to a product, more particularly to a protein or proteinaceous product, wherein said product is:
an antibody, or
a monoclonal antibody, more particularly the monoclonal antibody which is produced by the hybridoma deposited at the CNCM on Jun. 20, 2017 under the terms of the Budapest Treaty under accession number I-5211, or
a fragment of (monoclonal) antibody, which has retained the antigen binding specificity of said (monoclonal) antibody, such as a Fab, Fab′ or F(ab)2 fragment, or
a fusion protein, which has retained the antigen binding specificity of said (monoclonal) antibody, such as a scFv, or
a single-domain antibody (sdAb), or
the variable domain of a sdAb,
   wherein said product specifically binds to:
one of said soluble N-terminal ectodomain fragments and (sub-)fragments of (soluble) ectodomain fragments; or to
one of said C-terminal protein fragments and C-terminal ectodomain fragments.

CNCM is Collection Nationale de Culture de Microorganismes (Institut Pasteur; 28, rue du Docteur Roux; 75724 Paris CEDEX 15; France).

More particularly, the application also relates to a (proteinaceous) product, wherein said (proteinaceous) product specifically binds to one of said soluble N-terminal ectodomain fragments and (sub-)fragments of (soluble) ectodomain fragments, without binding to one of said C-terminal protein fragments and C-terminal ectodomain fragments.

More particularly, the application also relates to a product, more particularly to a protein or (proteinaceous) product, wherein said product specifically binds to one of said C-terminal protein fragments and C-terminal ectodomain fragments, without binding to one of said soluble N-terminal ectodomain fragments and (sub-)fragments of (soluble) ectodomain fragments.

The phrase “antibody” or “monoclonal antibody” includes conventional antibody (which comprises a heavy chain and a light chains) as well as single-domain antibody (sdAb; which, by contrast to a conventional antibody, is devoid of light chains and consists of a single monomeric variable antibody domain), such as a Heavy Chain Antibody (hcAb).

The phrase “antibody” or “monoclonal antibody” includes mono-, bi- or tri-specific antibodies.

The expression “fragment of (monoclonal) antibody, which has retained the antigen binding specificity” includes Fab, Fab′ and F(ab)2 fragments (of a conventional Ab or mAb), as well as the variable domain of a sdAb or hcAb (VHH or nanobody).

The expression “fusion protein, which has retained the antigen binding specificity of said (monoclonal) antibody” includes scFv.

The CDRs (or at least one of the CDR1, CDR2 and CDR3) of said antibody, antibody fragment or fusion protein may be the CDRs (or at least one of the CDR1, CDR2 and CDR3, respectively) of the monoclonal antibody which is produced by the hybridoma deposited on Jun. 20, 2017 at the CNCM under accession number I-5211.

Said antibody, antibody fragment or fusion protein may optionally be linked or bound to at least one detection label or marker or tag or drug.

The application also relates to drug-conjugated antibody to target HEMO-tumor cells, wherein said antibody is one of the antibodies herein defined.

Said HEMO tumor cells express at their surface a polypeptide, said polypeptide being one of said N-terminal (soluble) ectodomain fragments or one of said C-terminal protein fragments of the application.

The application also relates to a product, more particularly to a protein or proteinaceous product, wherein said product is:
an antibody, or
a monoclonal antibody, or
a fragment of (monoclonal) antibody, which has retained the antigen binding specificity of said (monoclonal) antibody, such as a Fab, Fab′ or F(ab)2 fragment, or
a fusion protein, which has retained the antigen binding specificity of said (monoclonal) antibody, such as a scFv, or
a single-domain antibody (sdAb), or
the variable domain of a sdAb,
   and wherein said product is optionally linked to at least one detection label or marker or tag or drug, and wherein said product specifically binds to a (human) HEMO antigen chosen from among the sequences of SEQ ID NOs: 8, 919, 924-939, 981-988 and 990.

The application also relates to a hybridoma, which produces a monoclonal antibody of the application. More particularly, the application relates to the hybridoma (2F7-E8) deposited on Jun. 20, 2017 at the CNCM under accession number I-5211 (which is directed against a fragment of the human HEMO ectodomain, i.e., a sub-fragment of a N-terminal soluble polypeptide of the application, i.e., fragment 123-286 from SEQ ID NO: 1 (fragment of SEQ ID NO: 8). More particularly, the application relates to the monoclonal antibody produced by the hybridoma (2F7-E8) deposited on Jun. 20, 2017 at the CNCM under accession number I-5211, possibly humanized, which is linked or bound to at least one detection label or marker or tag or drug. Said hybridoma (2F7-E8) or monoclonal antibody produced by the hybridoma (2F7-E8) deposited on Jun. 20, 2017 at the CNCM under accession number I-5211 is able to recognize a sub-fragment of a N-terminal soluble polypeptide of the application as well as the non-soluble form of said sub-fragment of a N-terminal soluble polypeptide of the application when the HEMO protein has not yet been shedding.

The application also relates to a Chimeric Antigen Receptor T cell (i.e., a CAR-T cell), wherein said Chimeric Antigen Receptor comprises an extracellular single-chain variable fragment (scFv) linked to an intracellular T Cell Receptor (TCR) signaling domain, and wherein said scFv is a scFv of the application.

Said intracellular TCR signaling domain may e.g., be CD3zeta.

Said scFv may be indirectly linked to said TCR signaling domain, e.g., via a hinge/spacer peptide and/or a transmembrane domain. The intracellular portion of the CAR may, in addition to said TCR signaling domain, comprise at least one costimulatory domain, such as CD28 or 4-1BB.

The application also relates to the antibody or monoclonal antibody of the application, or to the CAR-T cell of the application, for use in therapy, e.g., for use in anti-cancer treatment, more particularly in the treatment of a solid tumor.

Said antibody, monoclonal antibody, CAR-T cell may (specifically) bind to a tumor cell or to a tumor stem cell.

Said cancer is e.g., an ovarian cancer, an uterine cancer (more particularly an endometrial cancer, a cervical cancer, a gestational cancer (including placental cancer, e.g., choriocarcinoma)), a breast cancer, a lung cancer, a stomach cancer, a colon cancer, a liver cancer, a kidney cancer, a prostate cancer, an urothelial cancer, a germ cell cancer, a brain cancer, a head and neck cancer, a pancreatic cancer, a thyroid cancer, a thymus cancer, a skin cancer, a bone cancer or a bone marrow cancer. As urothelial cancers encompass carcinomas of the bladder, ureters and renal pelvis, said cancer may also be an urothelial cancer including a bladder cancer, an ureter cancer or a renal pelvis cancer.

The application also relates to the antibody or monoclonal antibody of the application, or to the CAR-T cell of the application, for use in diagnosis, more particularly in (in vitro) cancer diagnosis, more particularly in an (in vitro) method for determining the histotype, grade or stage of a tumor of a subject, or in an (in vitro) method for detecting a defect in the placentation of a pregnant subject, more particularly a placentation defect placing said pregnant subject at risk of placental abruption, pre-eclampsia or eclampsia.

The application also relates to the antibody or monoclonal antibody of the application, or to the CAR-T cell of the application, for use in an (in vitro) method for purifying or isolating circulating cells of a Boreoeutheria, more particularly for purifying or isolating circulating cells, which are tumor cells, or tumor stem cells, or placental cells, or in an (in vitro) method for inducing pluripotent stem cells from somatic cells.

The application also relates to the drug-conjugated (monoclonal) antibody of the application or to the CAR-T cell of the application, for use in therapy, more particularly for use in targeting tumoral cells in a Boreoeutheria suffering from tumor, wherein said tumoral cells expressed at their surface a polypeptide, said polypeptide being one of said N-terminal (soluble) ectodomain fragments or one of said C-terminal protein fragments of the application.

Said tumor is e.g., an ovarian tumor, an uterine tumor (more particularly an endometrial tumor, a cervical tumor, a gestational tumor (including placental tumor, e.g., choriocarcinoma)), a breast tumor, a lung tumor, a stomach tumor, a colon tumor, a liver tumor, a kidney tumor, a prostate tumor, an urothelial tumor, a germ cell tumor, a brain tumor, a head and neck tumor, a pancreatic tumor, a thyroid tumor, a thymus tumor, a skin tumor, a bone tumor or a bone marrow tumor. As urothelial tumors encompass carcinomas of the bladder, ureters and renal pelvis, said tumor may also be an urothelial tumor including a bladder tumor, a ureter tumor or a renal pelvis tumor.

The application also relates to a kit, which comprises product, more particularly to a protein or proteinaceous product, and which further comprises an instruction leaflet instructing to use said product in at least one of the following five uses:
the detection of the appearance of a tumor or the following of the evolution of a tumor in a subject, wherein said subject is a Boreoeutheria,
the determination of the histotype, grade or stage of a tumor, wherein said cancer is more particularly an ovarian cancer, an uterine cancer (more particularly an endometrial cancer, a cervical cancer, a gestational cancer (including placental cancer, e.g., choriocarcinoma)), a breast cancer, a lung cancer, a stomach cancer, a colon cancer, a liver cancer, a kidney cancer, a prostate cancer, an urothelial cancer (including bladder cancer, ureter cancer or renal pelvis cancer), a germ cell cancer, a brain cancer, a head and neck cancer, a pancreatic cancer, a thyroid cancer, a thymus cancer, a skin cancer, a bone cancer or a bone marrow cancer,
the detection of a defect in the placentation of a pregnant subject, more particularly a placentation defect placing said pregnant subject at risk of placental abruption, pre-eclampsia or eclampsia,
the purification or isolation of circulating cells of a Boreoeutheria, more particularly for purifying or isolating circulating cells, which are tumor cells, or tumor stem cells, or placental cells,
the purification or isolation of non-circulating cells of a Boreoeutheria, more particularly for purifying or isolating non-circulating cells in a fresh tumor or biopsy sample from a Boreoeutheria,
   wherein said (proteinaceous) product is the (proteinaceous) product of the application, and
   wherein said (proteinaceous) product is optionally linked or bound to a detection label.

The application also relates to a nucleic acid, which codes a polypeptide, wherein said polypeptide consists of one of said soluble N-terminal ectodomain fragments, said (sub-)fragments of (soluble) ectodomain fragments, said C-terminal protein fragments and said C-terminal ectodomain fragments. Said nucleic acid may be a DNA, a RNA, or a cDNA.

A nucleic acid coding for human HEMO is the sequence of SEQ ID NO: 152. A nucleic acid coding for non-human HEMO can be chosen from among the sequences of SEQ ID NO: 153-167.

The application relates more particularly to the fragments of the sequences of SEQ ID NO: 152 and 153-167, which codes for a polypeptide, wherein said polypeptide consists of one of said soluble N-terminal ectodomain fragments, said (sub-)fragments of (soluble) ectodomain fragments, said C-terminal protein fragments and said C-terminal ectodomain fragments.

The application also relates to a nucleic acid vector, more particularly to a nucleic acid expression vector, which (recombinantly) comprises at least one nucleic acid of the application.

The application also relates to an engineered host cell, which (recombinantly) comprises at least one nucleic acid or vector of the application.

The application also relates to a nucleic acid probe, which specifically hybridizes to a nucleic acid of the application.

The application also relates to a primer pair, which specifically amplifies at least one of the nucleic acids of the application.

The application also relates to the HEMO promoter, more particularly to the human HEMO promoter, more particularly to the human HEMO promoter of SEQ ID NO: 669.

The application also relates to a kit, which comprises at least one (proteinaceous) product of the application, or at least probe, primer pair or set of oligonucleotides of the application, wherein said kit optionally further comprises an instruction leaflet for use of the kit in the detection of shed forms of the HEMO protein and/or in the detection of tumor cells, of stem cells or of tumor stem cells.

The application also relates to a solid support, such a membrane or a chip, onto which at least one (proteinaceous) product of the application, or at least probe, primer pair or set of oligonucleotides of the application is bound, linked or attached.

The application also relates to any composition, pharmaceutical composition or drug, which comprises at least one product of the application, and which optionally further comprises at least one buffer or pharmaceutically acceptable vehicle (or diluent or adjuvant).

The application relates more particularly to such a pharmaceutical composition or drug, wherein said at least one product of the application is at least one antibody, monoclonal antibody or CAR-T cell of the application. Such a pharmaceutical composition or drug might be useful e.g., in the treatment of cancer, more particularly of an ovarian cancer, an uterine cancer (more particularly an endometrial cancer, a cervical cancer, a gestational cancer (including placental cancer, e.g., choriocarcinoma)), a breast cancer, a lung cancer, a stomach cancer, a colon cancer, a liver cancer, a kidney cancer, a prostate cancer, an urothelial cancer, a germ cell cancer, a brain cancer, a head and neck cancer, a pancreatic cancer, a thyroid cancer, a thymus cancer, a skin cancer, a bone cancer or a bone marrow cancer. As urothelial cancers encompass carcinomas of the bladder, ureters and renal pelvis, said cancer may also be an urothelial cancer including a bladder cancer, an ureter cancer or a renal pelvis cancer.

According to a particular embodiment, the invention relates to an isolated cell or part of a cell (e.g. exosome), which expresses the C-terminal protein fragments and C-terminal ectodomain fragments herein defined, wherein a portion of the C-terminal protein fragments and C-terminal ectodomain fragments herein defined is expressed at the surface of said cell or said part of said cell, and wherein said surface-expressed portion comprises the C-terminal fragment of ectodomain which is comprised in said the C-terminal protein fragments and C-terminal ectodomain fragments herein defined,

more particularly wherein said cell or said part of said cell is a placental cell or a part of a placental cell, a stem cell or a part of a stem cell, a tumor cell or a part of a tumor cell, or a tumor stem cell or a part of a tumor stem cell.

According to a particular embodiment, the invention relates to an isolated cell, which expresses the C-terminal protein fragments and C-terminal ectodomain fragments herein defined, wherein a portion of the C-terminal protein fragments and C-terminal ectodomain fragments herein defined is expressed at the surface of said cell, and wherein said surface-expressed portion comprises the C-terminal fragment of ectodomain which is comprised in said the C-terminal protein fragments and C-terminal ectodomain fragments herein defined, more particularly wherein said cell is a placental cell, a stem cell, a tumor cell or a tumor stem cell.

According to another particular embodiment, the invention relates to the soluble N-terminal ectodomain fragments and (sub-)fragments of (soluble) ectodomain fragments herein defined, for use in therapy, more particularly for use in the treatment of a defect in placentation of a Boreoeutheria or in the treatment of a defect in the protection against microbial infection, more particularly viral infection, of a fetus carried by a Boreoeutheria.

According to another particular embodiment, the invention also relates to a product, wherein said product is:
a. an antibody, or
b. a monoclonal antibody, more particularly the monoclonal antibody which is produced by the hybridoma deposited at the CNCM under accession number I-5211, or
c. a Fab, Fab′ or F(ab)2 fragment, or
d. a scFv, or
e. a sdAb, or
f. the variable domain of a sdAb,
   and wherein said product is optionally linked to at least one drug,
   and wherein said product specifically binds to the said soluble N-terminal ectodomain fragments and (sub-)fragments of (soluble) ectodomain fragments herein defined.

According to another particular embodiment, the invention also relates to product, wherein said product is:
a. an antibody, or
b. a Fab, Fab′ or F(ab)2 fragment, or
c. a scFv, or
d. a sdAb, or
e. the variable domain of a sdAb,
   and wherein said product is optionally linked to at least one drug,
   and wherein said product specifically binds to the said C-terminal protein fragments and C-terminal ectodomain fragments herein defined.

According to another particular embodiment, the invention relates to a chimeric Antigen Receptor T cell (CAR-T cell), wherein said Chimeric Antigen Receptor comprises a scFv linked to a TCR signaling domain, and wherein said scFv is the scFv of said product herein defined.

More particularly, the invention relates to said products herein defined, or the CAR-T cell herein defined, for use in therapy, more particularly in anti-cancer therapy, wherein said product or CAR-T cell binds to a tumor cell or to a tumor stem cell, and wherein said cancer is more particularly an ovarian cancer, an uterine cancer, a cervical cancer, a gestational cancer, a breast cancer, a lung cancer, a stomach cancer, a colon cancer, a liver cancer, a kidney cancer, a prostate cancer, an urothelial cancer (including bladder cancer, ureter cancer or renal pelvis cancer), a germ cell cancer, a brain cancer, a head and neck cancer, a pancreatic cancer, a thyroid cancer, a thymus cancer, a skin cancer, a bone cancer or a bone marrow cancer.

More particularly, the invention also relates to a kit which comprises a product, wherein said product is used in at least one of the following five uses:
a. the detection of the appearance of a tumor or the following of the evolution of a tumor in a subject, wherein said subject is a Boreoeutheria;
   and/or
b. the determination of the histotype, grade or stage of a tumor, wherein said cancer is an ovarian cancer, an uterine cancer, a cervical cancer, a gestational cancer, a breast cancer, a lung cancer, a stomach cancer, a colon cancer, a liver cancer, a kidney cancer, a prostate cancer, an urothelial cancer (including bladder cancer, ureter cancer or renal pelvis cancer), a germ cell cancer, a brain cancer, a head and neck cancer, a pancreatic cancer, a thyroid cancer, a thymus cancer, a skin cancer, a bone cancer or a bone marrow cancer;
   and/or
c. the detection of a defect in the placentation of a pregnant subject, more particularly a placentation defect placing said pregnant subject at risk of placental abruption, pre-eclampsia or eclampsia;
   and/or
d. the purification or isolation of circulating cells of a Boreoeutheria, more particularly for purifying or isolating Boreoeutheria circulating cells, which are tumor cells, or tumor stem cells, or placental cells,
   and/or
e. the purification or isolation of non-circulating cells of a Boreoeutheria, more particularly for purifying or isolating Boreoeutheria non-circulating cells in a biopsy or tumor samples from said Boreoeutheria, and
   wherein said (proteinaceous) product is the (proteinaceous) product of the application, and
   wherein said (proteinaceous) product is optionally linked or bound to a detection label.

### Methods

The application relates to methods, which involves or implements at least one product of the application, more particularly at least one of said soluble N-terminal ectodomain fragments, said (sub-)fragments of (soluble) ectodomain fragments, said C-terminal protein fragments, said C-terminal ectodomain fragments, said cells, said (proteinaceous) products, and said probes and/or primers.

The methods of the application notably comprise methods of:
tumor typing,
cancer diagnostic,
cancer immunotherapy,
screening for therapeutic agents, e.g., screening for agents, which may be useful in the treatment of cancer (including palliation or prevention of cancer), or for agents, which may be useful in the treatment of a defect in placenta development (e.g., placental abruption, pre-eclampsia, eclampsia), or for agents, which may be useful in fetus protection (e.g., protection against viral or microbial infection),
purification of circulating cells (e.g., purification of tumoral circulating cells, or of circulating trophoblasts), and
production of induced pluripotent stem cells (from somatic cells).

More particularly, the application relates to an (in vitro) method for detecting that tumor cells or tumor stem cells are present in a subject, wherein said subject is a Boreoeutheria, and wherein said in vitro method comprises at least one of the following two steps i. and ii.:

i. detecting a polypeptide which is contained in soluble form in a sample, wherein said polypeptide in soluble form is one of the soluble N-terminal ectodomain fragments of the application, wherein said sample is a blood sample or an urine sample or an ascites liquid sample from said subject, or a biopsy sample of a tissue from said subject, or a protein extract of said blood sample or urine sample or ascites liquid sample or biopsy sample (more particularly a soluble protein extract of said blood sample or urine sample or biopsy sample), and wherein detecting said polypeptide in soluble form in said sample is indicative that tumor cells or tumor stem cells are present in said subject; and
ii. detecting cells in a sample, wherein said cells are or comprises the cells of the application (which express at least one of said C-terminal ectodomain fragments at their surface), wherein said sample is a blood sample or an urine sample or an ascites liquid sample from said subject, a biopsy sample of a tissue from said subject, or the cell fraction of said blood or urine or ascites liquid or biopsy sample, and wherein detecting said cells in said sample is indicative that tumor cells or tumor stem cells are present in said subject.

The application also relates to an (in vitro) method for detecting the appearance of a tumor or for following the evolution of a tumor in a subject, wherein said subject is a Boreoeutheria, and wherein said (in vitro) method comprises at least one of the following two steps i. and ii.:

i. detecting a polypeptide which is contained in soluble form in a sample, wherein said polypeptide in soluble form is one of the soluble N-terminal ectodomain fragments of the application, wherein said sample is a blood sample or an urine sample or an ascites liquid sample from said subject, or a biopsy sample of a tissue from said subject, or a protein extract of said blood sample or urine sample or ascites liquid sample or biopsy sample (more particularly a soluble protein extract of said blood sample or urine sample or biopsy sample), and wherein detecting said polypeptide in soluble form in said sample is indicative that tumor cells or tumor stem cells are present in said subject; and
ii. detecting cells in a sample, wherein said cells are or comprises the cells of the application (which express at least one of said C-terminal ectodomain fragments at their surface), wherein said sample is a blood sample or an urine sample or an ascites liquid sample from said subject, a biopsy sample of a tissue from said subject, or the cell fraction of said blood or urine or ascites liquid or biopsy sample, and wherein detecting said cells in said sample is indicative that tumor cells or tumor stem cells are present in said subject.

The expression “following the evolution of a tumor in a subject” encompasses the detection of the appearance of said tumor or the detection of the reappearance of said tumor after treatment and the determining of the histotype, grade or stage of said tumor before treatment and during treatment.

More particularly, the application relates to an (in vitro) method herein defined for detecting the appearance of a tumor secreting one of the soluble N-terminal ectodomain fragments of the application at a concentration higher than the average concentration measured in a sample of a control subject.

More particularly, the application relates to an (in vitro) method herein defined for detecting the reappearance of tumor after treatment.

Said tumor may e.g., be an ovarian tumor, an uterine tumor (more particularly an endometrial tumor, a cervical tumor, a gestational tumor (including placental tumor, e.g., choriocarcinoma)), a breast tumor, a lung tumor, a stomach tumor, a colon tumor, a liver tumor, a kidney tumor, a prostate tumor, an urothelial tumor, a germ cell tumor, a brain tumor, a head and neck tumor, a pancreatic tumor, a thyroid tumor, a thymus tumor, a skin tumor, a bone tumor or a bone marrow tumor. As urothelial tumors encompass carcinomas of the bladder, ureters and renal pelvis, said tumor may also be an urothelial tumor including a bladder tumor, an ureter tumor or a renal pelvis tumor.

The application also relates to an (in vitro) method for determining the histotype, grade or stage of a tumor of a subject, wherein said subject is a Boreoeutheria, and wherein said in vitro method comprises at least one of the following two steps i. and ii.:

i. detecting a polypeptide which is contained in soluble form in a sample, wherein said polypeptide in soluble form is one of the soluble N-terminal ectodomain fragments of the application, wherein said sample is a blood sample or an urine sample or an ascites liquid sample from said subject, or a biopsy sample of said tumor, or a protein extract of said blood sample or urine sample or ascites liquid simple or tumor biopsy sample (more particularly a soluble protein extract of said blood sample or urine sample or tumor biopsy sample), and wherein detecting said polypeptide in soluble form in said sample determines the histotype, grade or stage of said tumor; and
ii. detecting cells in a sample, wherein said cells are or comprise the cells of the application (which express at least one of said C-terminal ectodomain fragments at their surface), wherein said sample is a blood sample or an urine sample or an ascites liquid sample from said subject, a biopsy sample of said tumor, or the cell fraction of said blood or urine or ascites liquid or biopsy sample, and wherein detecting said cells in said sample determines the histotype, grade or stage of said tumor.

More particularly, the application relates to an (in vitro) method for determining the histotype, grade or stage of a tumor of a subject, wherein said subject is a Boreoeutheria, and wherein said in vitro method comprises detecting cells in a sample, wherein said cells are or comprise the cells of the application (which express at least one of said C-terminal ectodomain fragments at their surface), wherein said sample is a biopsy sample of said tumor, or the cell fraction of said biopsy sample, and wherein detecting said cells in said sample determines the histotype, grade or stage of said tumor.

Detecting said polypeptide in soluble form or detecting said cells may comprise measuring the quantity or concentration of said polypeptide or cells, respectively, and optionally comparing the measured quantity or concentration to a reference quantity or concentration (e.g., a control quantity or concentration).

Said tumor may e.g., be an ovarian tumor, an uterine tumor (more particularly an endometrial tumor, a cervical tumor, a gestational tumor (including placental tumor, e.g., choriocarcinoma)), a breast tumor, a lung tumor, a stomach tumor, a colon tumor, a liver tumor, a kidney tumor, a prostate tumor, an urothelial tumor, a germ cell tumor, a brain tumor, a head and neck tumor, a pancreatic tumor, a thyroid tumor, a thymus tumor, a skin tumor, a bone tumor or a bone marrow tumor. As urothelial tumors encompass carcinomas of the bladder, ureters and renal pelvis, said tumor may also be an urothelial tumor including a bladder tumor, an ureter tumor or a renal pelvis tumor.

A concentration (significantly) higher than
the average concentration measured in the blood of a control subject (control subject with no cancer and no tumor), or
the average concentration routinely measured in the blood of a subject
   might be indicative of the presence of a tumor in said subject. The average concentration measured in the blood of a control subject (with no cancer/no tumor) may e.g., be of 1 fM-1 mM or 1 fM-1 μM or 1 fM-1 nM or 1 fM-1 pM or 1 pM-1 mM or 1 pM-1 μM or 1 pM-1 nM or 1 nM-1 mM or 1 nM-1 μM or 1 μM-1 mM or 1 fM-100 pM or 1 fM-10 pM or 1 pM-100 nM or 1 pM-10 nM or 1 nM-100 μM or 1 nM-10 μM or 1 μM-100 mM or 1 μM-10 mM or 1 pM-10 nM or 1 pM-10 pM or 1 pM-100 pM or 100 pM-10 nM or 1 nM-100 nM or 1 nM-10 nM or 10 nM-100 nM or 5 nM-100 nM or 1 nM-5 nM. A concentration outside these normal ranges herein and higher than the superior extremity of said ranges, e.g. 1 mM or 1 μM or 1 nM, or 1 pM or 100 nM or 100 pM or 10 nM or 10 pM or 5 nM, may be indicative of the presence of a tumor in said subject.

The tumor histotype, grade or stage, which has been thus determined, may guide the physician in selecting the anti-tumor treatment and/or in adjusting the anti-tumor treatment. The application thus relates to a method for selecting an anti-cancer treatment for a subject in need thereof, which comprises determining the histotype, grade or stage of said tumor using the histotyping/grading/staging method of the application, and selecting among the anti-cancer treatments by surgery, chimiotherapy, radiotherapy and hormonotherapy, a treatment, which is adapted to said tumor histotype, grade or stage.

The application also relates to an (in vitro) method for detecting a defect in the placentation of a pregnant subject, more particularly a placentation defect placing said pregnant subject at risk of placental abruption, pre-eclampsia or eclampsia, wherein said subject is a Boreoeutheria, and wherein said in vitro method comprises at least one of the following two steps i. and ii.:

i. measuring the quantity or concentration of a polypeptide in soluble form in a sample, wherein said polypeptide in soluble form is one of the soluble N-terminal ectodomain fragments of the application, wherein said sample is a blood sample or an urine sample or an amniotic liquid sample from said subject or a (soluble) protein extract of said blood or urine or amniotic liquid sample, and wherein (measuring) said quantity or concentration in said sample is indicative of a defect in the placentation of said subject; and
ii. measuring the quantity or concentration of cells in a sample, wherein said cells are the cells of the application (which express at least one of said C-terminal ectodomain fragments at their surface), wherein said sample is a blood sample or an urine sample or an amniotic liquid sample from said Boreoeutheria or a placenta sample from said Boreoeutheria or a cell extract from said blood or urine or amniotic liquid or placenta sample, and wherein (measuring) said quantity or concentration in said sample is indicative of a defect in the placentation of said subject.

More particularly, the application relates to an in vitro method for detecting a defect in the placentation of a pregnant subject (e.g., placental abruption, pre-eclampsia, eclampsia), wherein said subject is a Boreoeutheria, wherein said in vitro method comprises measuring the quantity or concentration of a polypeptide in soluble form in a sample, wherein said polypeptide in soluble form is one of the soluble N-terminal ectodomain fragments of the application, wherein said sample is a blood sample from said Boreoeutheria or a (soluble) protein extract from said blood sample, and wherein (measuring) said quantity or concentration in said sample is indicative of a defect in the placentation of said subject.

A concentration (significantly) higher or lower than the average concentration measured in the blood of a control subject (pregnant control subject with normal placentation) might be indicative of a defect in the placentation of said subject.

The average concentration measured in the blood of a control subject (pregnant control subject with normal placentation) may e.g., be of be of 1 fM-1 mM or 1 fM-1 μM or 1 fM-1 nM or 1 fM-1 pM or 1 pM-1 mM or 1 pM-1 uM or 1 pM-1 nM or 1 nM-1 mM or 1 nM-1 μM or 1 μM-1 mM or 1 fM-100 pM or 1 fM-10 pM or 1 pM-100 nM or 1 pM-10 nM or 1 nM-100 μM or 1 nM-10 μM or 1 μM-100 mM or 1 μM-10 mM or 1 pM-10 nM or 1 pM-10 pM or 1 pM-100 pM or 100 pM-10 nM or 1 nM-100 nM or 1 nM-10 nM or 10 nM-100 nM or 5 nM-100 nM or 1 nM-5 nM, particularly of 1 nM-10 nM (at e.g., the third trimester of pregnancy). A concentration outside this normal range at e.g., the third trimester of pregnancy (for example a concentration, which at the third trimester of pregnancy is below said normal range), may be indicative of a defect in the placentation of said subject.

The application also relates to an (in vitro) method for testing a Boreoeutheria for pregnancy, which comprises

i. measuring the quantity or concentration of a polypeptide in soluble form in a blood sample from said subject, wherein said polypeptide in soluble form is one of the soluble N-terminal ectodomain fragments of the application, and wherein measuring said quantity or concentration in said sample is indicative of the pregnancy or non-pregnancy of said Boreoeutheria; and
ii. measuring the quantity or concentration of cells in a blood sample from said subject, wherein said cells are the cells of the application (which express at least one of said C-terminal ectodomain fragments at their surface), and wherein measuring said quantity or concentration in said sample is indicative of the pregnancy or non-pregnancy of said Boreoeutheria.

A quantity or concentration (significantly) higher or lower than the average quantity or concentration measured in the blood of a control subject (non-pregnant control subject) might be indicative of said Boreoeutheria being pregnant or non-pregnant.

The application also relates to an (in vitro) method for detecting a defect in the protection of a fetus against microbial (more particularly viral) infection in a pregnant Boreoeutheria, wherein said in vitro method comprises at least one of the following two steps i. and ii.:

i. measuring the quantity or concentration of a polypeptide in soluble form in a blood sample from said pregnant Boreoeutheria, wherein said polypeptide in soluble form is one of the soluble N-terminal ectodomain fragments of the application, and wherein measuring said quantity or concentration in said sample is indicative of a defect in the protection of said fetus against microbial (more particularly viral) infection; and
ii. measuring the quantity or concentration of cells or part of cell (e.g. exosome) in a blood sample from said pregnant Boreoeutheria, wherein said cells are the cells of the application (which express at least one of said C-terminal ectodomain fragments at their surface), [optionally determining by genetic analysis whether said cells are maternal cells or fetal cells], and wherein measuring said quantity or concentration in said sample is indicative of a defect in the protection of said fetus against microbial (more particularly viral) infection.

A quantity or concentration (significantly) higher or lower than the average concentration measured in the blood of a control subject (pregnant control subject with normal placentation) might be indicative of a defect in the placentation of said subject.

The application also relates to an (in vitro) method for determining whether a compound is a candidate active principle for therapy in a Boreoeutheria, wherein said therapy is the treatment of a defect in placentation of said Boreoeutheria or the treatment of a defect in the protection against microbial (more particularly viral) infection of a fetus carried by said Boreoeutheria, wherein said (in vitro) method comprises placing said compound in contact with a ligand of one of the soluble N-terminal ectodomain fragments of the application to perform a ligand binding assay, and detecting whether said compound binds to said ligand, wherein detecting said binding is indicative that said compound is a candidate active principle for said therapy.

In addition to being placed in contact with a ligand of one of the soluble N-terminal ectodomain fragments of the application, said compound can be placed in contact with one of the soluble N-terminal ectodomain fragments of the application to perform a competitive binding assay. Detecting competition between said compound and said polypeptide for binding to said ligand may be indicative that said compound is a candidate active principle for said therapy.

Said ligand may e.g., be a monoclonal antibody or scFv of the application.

The application also relates to an (in vitro) method for determining whether a compound is a candidate active principle for therapy in a Boreoeutheria, wherein said therapy is the treatment of a cancer in said Boreoeutheria, wherein said method comprises

placing said compound in contact with one of the soluble N-terminal ectodomain fragments of the application to perform a polypeptide binding assay, and detecting whether said compound binds to said polypeptide, wherein detecting said binding is indicative that said compound is a candidate active principle for said therapy.

In addition to being placed in contact with one of the soluble N-terminal ectodomain fragments of the application, said compound can be placed in contact with a ligand of said polypeptide to perform a competitive binding assay. Detecting competition between said compound and said ligand for binding to said polypeptide may be indicative that said compound is a candidate active principle for said therapy.

Said ligand may e.g., be a monoclonal antibody or scFv of the application.

The application also relates to an (in vitro) method for purifying or isolating circulating cells of a Boreoeutheria, wherein said method comprises purifying or isolating cells from a sample of circulating blood of said Boreoeutheria or from the cell fraction of such a sample, wherein said cell purification or isolation comprises positively sorting cells, which express a cell marker at their surface, and wherein said cells are the cells of the application (which express at least one of said C-terminal ectodomain fragments at their surface).

More particularly, the application also relates to an (in vitro) method for purifying or isolating circulating cells of a Boreoeutheria, more particularly for purifying or isolating Boreoeutheria circulating cells, which are tumor cells, or tumor stem cells, or placental cells, wherein said method comprises purifying or isolating cells from a sample of circulating blood of said Boreoeutheria or from the cell fraction of such a sample, wherein said cell purification or isolation comprises positively sorting cells that bind to a ligand, wherein said ligand is a (proteinaceous) product of the application.

Said positive sorting can be e.g., performed using a ligand, which specifically binds to a polypeptide that is expressed at the surface of said circulating cells, and wherein said polypeptide is one of said N-terminal (soluble) ectodomain fragments or one of said C-terminal protein fragments and C-terminal ectodomain fragments, more particularly one of said C-terminal protein fragments and C-terminal ectodomain fragments.

Said circulating cells may e.g., be tumor cells, or tumor stem cells, or placental cells.

The application also relates to an (in vitro) method for purifying or isolating non-circulating (tumoral) cells in a fresh tumor or biopsy sample from a Boreoeutheria to characterize the said non-circulating (tumoral) cells (with, e.g., RNAseq or DNAseq or PDXmice techniques), wherein said non-circualting (tumoral) cells purification or isolation comprises positively sorting cells that bind to a ligand, wherein said ligand is a (proteinaceous) product of the application.

The application also relates to an (in vitro) method for inducing pluripotent stem cells from somatic cells, which comprises introducing pluripotency-associated genes into somatic cells (e.g., into fibroblasts), and selecting those cells, which express the introduced pluripotency-associated genes, wherein said pluripotency-associated genes comprises a gene coding for a polypeptide, which consists of one of the soluble N-terminal ectodomain fragments of the application.

Said pluripotency-associated genes may further comprise one or several genes, which code for a transcription factor, for example one or several genes chosen from among the genes coding for the Oct4 (Pou5f1), Sox, Klf, Myc, Nanog, LIN28 and Glis1 transcription factors, for example one or several genes chosen from among the genes coding for the Oct4 (Pou5f1), Sox2, cMyc, and Klf4 transcription factors. Said pluripotency-associated genes can be carried on one or several viral vectors, more particularly on one or several retroviruses.

Said method may further comprise growing the selected cells in a cell culture medium. Said cell culture medium may comprise one or several components chosen from among basic Fibroblast Growth Factor (bFGF), cytokines (such as Tumor Growth Factor (TGF) or Wnt3a), Fetal Bovine Serum (FBS), human serum, collagen, albumin, cholesterol and insulin.

The application also relates to an (in vitro) method for detecting the soluble N-terminal ectodomain fragments and (sub-)fragments of (soluble) ectodomain fragments herein defined in a sample from a subject, which comprises or consists in an ELISA sandwich assay using at least:
a first monoclonal or polyclonal antibody directed against a first epitope of said soluble N-terminal ectodomain fragments or said (sub-)fragments of (soluble) ectodomain fragments, and
a second monoclonal or polyclonal antibody directed against a second epitope of said soluble
N-terminal ectodomain fragments or said (sub-)fragments of (soluble) ectodomain fragments,
   said first and second epitopes being different and wherein said sample is a blood or an urine or an amniotic liquid or an ascites liquid sample from said subject or a (soluble) protein extract of said blood or urine or amniotic liquid or ascites liquid sample.

For example, such an ELISA assay can be used to detect variability in the HEMO sera level, in normal and pathological conditions and/or follow the evolution in pathological conditions.

The term “comprising”, which is synonymous with “including” or “containing”, is open-ended, and does not exclude additional, un-recited element(s), ingredient(s) or method step(s), whereas the term “consisting of” is a closed term, which excludes any additional element, step, or ingredient which is not explicitly recited.

The term “essentially consisting of” is a partially open term, which does not exclude additional, un-recited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

According to a particular embodiment, the invention relates to an in vitro method for detecting the soluble N-terminal ectodomain fragments and (sub-)fragments of (soluble) ectodomain fragments herein defined or the cells herein defined which express the C-terminal protein fragments and C-terminal ectodomain fragments, which comprises at least one of the following two steps a. and b.:
a. detecting a polypeptide which is contained in soluble form in a sample, wherein said polypeptide is the soluble N-terminal ectodomain fragments and (sub-)fragments of (soluble) ectodomain fragments herein defined;
   and/or
b. detecting cells in a sample, wherein said cells are or comprise the cells herein defined which express the C-terminal protein fragments and C-terminal ectodomain fragments.

According to another particular embodiment, the invention relates to the above in vitro method for determining the histotype, grade or stage of a tumor of a subject, wherein said subject is a Boreoeutheria, and wherein said in vitro method comprises at least one of the following two steps a. and b.:
a. detecting a polypeptide which is contained in soluble form in a sample, wherein said polypeptide is the soluble N-terminal ectodomain fragments and (sub-)fragments of (soluble) ectodomain fragments herein defined and wherein said sample is:
   a blood or urine or ascites liquid sample from said subject, or
   a biopsy sample of said tumor,
   or a soluble protein extract of said blood or urine or ascites liquid or tumor biopsy sample, and
      wherein detecting said polypeptide in soluble form in said sample determines the histotype, grade or stage of said tumor;
      and/or
b. detecting cells in a sample, wherein said cells are or comprise the cells herein defined which express the C-terminal protein fragments and C-terminal ectodomain fragments and wherein said sample is:
   a blood or urine or ascites liquid sample from said subject, or
   a biopsy sample of said tumor, or
   the cell fraction of said blood or urine or ascites liquid or biopsy sample, and
      wherein detecting said cells in said sample determines the histotype, grade or stage of said tumor.

According to another particular embodiment, the invention relates to the above in vitro method for detecting a defect in the placentation of a pregnant subject, more particularly a placentation defect placing said pregnant subject at risk of placental abruption, pre-eclampsia or eclampsia, wherein said subject is a Boreoeutheria, and wherein said in vitro method comprises at least one of the following two steps a. and b.:
a. measuring the quantity or concentration of a polypeptide in soluble form in a sample, wherein said polypeptide in soluble form is the soluble N-terminal ectodomain fragments and (sub-)fragments of (soluble) ectodomain fragments herein defined and wherein said sample is:
   a blood or urine or amniotic liquid sample from said subject,
   or a soluble protein extract of said blood or urine or amniotic liquid sample, and
      wherein said quantity or concentration in said sample is indicative of a defect in the placentation of said subject;
      and/or
b. measuring the quantity or concentration of cells in a sample, wherein said cells are the cells herein defined which express the C-terminal protein fragments and C-terminal ectodomain fragments and wherein said sample is:
   a blood or urine or amniotic liquid sample from said Boreoeutheria,
   or a placenta sample from said Boreoeutheria,
   or a cell extract from said blood or urine or amniotic liquid or placenta sample, and
      wherein said quantity or concentration in said sample is indicative of a defect in the placentation of said subject.

According to another particular embodiment, the invention relates to an in vitro method for purifying or isolating circulating cells of a Boreoeutheria, more particularly for purifying or isolating Boreoeutheria circulating cells, which are tumor cells, or tumor stem cells, or placental cells,

wherein said method comprises purifying or isolating cells from a sample of circulating blood of said Boreoeutheria or from the cell fraction of such a sample, wherein said cell purification or isolation comprises positively sorting cells that bind to a ligand, wherein said ligand is the product herein defined.

According to another particular embodiment, the invention relates to an in vitro method for purifying or isolating non-circulating cells in a fresh tumor or biopsy sample from a Boreoeutheria to characterize the said non-circulating cells,

wherein said non-circulating cells purification or isolation comprises positively sorting cells that bind to a ligand, wherein said ligand is a (proteinaceous) product of the application.

According to another particular embodiment, the invention relates to an in vitro method for inducing pluripotent stem cells from somatic cells, which comprises introducing pluripotency-associated genes into somatic cells, and selecting those cells, which express the introduced pluripotency-associated genes,

wherein said pluripotency-associated genes comprises a gene coding for a polypeptide, which consists of the soluble N-terminal ectodomain fragments and (sub-)fragments of (soluble) ectodomain fragments herein defined and/or of the C-terminal protein fragments and C-terminal ectodomain fragments herein defined.

### EXAMPLES

### Example 1

Capture of retroviral envelope genes has been pivotal to the emergence of placental mammals, with evidence for multiple, reiterated and independent capture events occurring in mammals and responsible for the diversity of present-day placental structures. Here we uncover a full-length endogenous retrovirus envelope protein with unprecedented characteristics as it is actively shed in the blood circulation in humans, via specific cleavage of the precursor envelope protein upstream of the transmembrane domain. At variance with previously identified retroviral envelope genes, its encoding gene is found to be transcribed from a unique CpG-rich promoter not related to a retroviral LTR, with sites of expression including the placenta as well as other tissues, and rather unexpectedly stem cells as well as reprogrammed iPS cells where the protein can also be detected. We provide evidence that the associated retroviral capture event most probably occurred >100 Mya, before the split of Laurasiatheria and Euarchontoglires, with the identified retroviral envelope gene encoding a full-length protein in all simians, under purifying selection and with similar shedding capacity. Finally a comprehensive screen of the expression of the gene discloses high transcript levels in several tumor tissues such as germ cell, breast and ovarian tumors, with in the latter case evidence for a histotype dependence and specific protein expression in clear-cell carcinoma. Conclusively, the identified protein is likely to constitute a “stemness marker” of the normal cell, and a “target” for immunotherapeutic approaches in definite tumors.

### Significance

Endogenization of retroviruses is a rare but common event in vertebrates, with the captured retroviral envelope syncytins playing a major role in placentation in mammals—including marsupials. Here we identify an endogenous retroviral envelope protein with unprecedented properties, including a specific cleavage process resulting in the shedding of its extracellular moiety in the human blood circulation. This protein is conserved in all simians—with a homologous protein found in marsupials—with a “stemness” expression in embryonic and reprogrammed stem cells, as well as in the placenta and some human tumors, especially ovarian tumors. This protein is likely to constitute a versatile marker—and possibly an effector—of specific cellular states, and, being shed, can be immuno-detected in the blood.

### Methods

### Biological Samples

First trimester human placenta tissues were obtained from legal elective terminations of pregnancy (gestational age 8 to 12 weeks), with parent's written informed consent, from the Department of Obstetrics and Gynecology at the COCHIN Hospital, Paris 75014, France.

All blood samples were obtained with written informed consent. Samples from pregnant (11 to 18 weeks of amenorrhea) and non-pregnant (before ovulation induction hormonal therapy) women were from Laboratoire EYLAU (34, avenue du Roule; 92200 Neuilly sur Seine; France) under MTA protocol MTA2015-45. Male blood samples were from ETABLISSEMENT FRANÇAIS DU SANG (20 Avenue du Stade de France; 93218 Saint-Denis; France) under agreement 15EFS018.

Ovary tissue samples were from the Biological Resource Centre and the Department of Laboratory Medicine and Pathology of the GUSTAVE ROUSSY INSTITUTE (114, rue Edouard Vaillant; 94800 Villejuif; France) under Research Agreement RT09916.

RNAs from hESC (H1, H7, H9) were from U1170-INSERM of the GUSTAVE ROUSSY INSTITUTE.

iPSC (reprogrammed CD34+ human cells, at passage 24) and their supernatant were from the iPSC Platform of the GUSTAVE ROUSSY INSTITUTE.

The source of non-human primate genomic DNAs is described in Esnault et al. 2013, and the source of Wallaby RNA in Cornelis et al. 2015.

### Ethics Statement

Experiments were approved by the Ethics Committee of the GUSTAVE ROUSSY INSTITUTE. This study was carried out in strict accordance with the French and European laws and regulations regarding Animal Experimentation (Directive 86/609/EEC regarding the protection of animals used for experimental and other scientific purposes).

### Polyclonal and Monoclonal Anti-HEMO Antibodies

A DNA fragment coding for 163 amino acids of the HEMO SU envelope subunit (aa 123 to 286; SEQ ID NO: 8) was inserted into the pET28b (NOVAGEN) prokaryotic expression vector and expressed in BL21(DE3) bacteria. The recombinant C-term-His-tagged protein was purified from bacteria lysates by nickel affinity chromatography. Mice immunization was performed in accordance with standard procedures. Sera containing polyclonal antibodies were recovered independently from 10 mice and tested by Western blot analyses using lysates of 293T cells transiently transfected with HEMO Env expression vector. One mouse was selected for monoclonal antibody production by AGRO-BIO (2 allée de la Chavannerie; 45240 La Ferté Saint-Aubin; France), and one hybridoma clone was isolated (2F7, IgG2a isotype) for IgG production.

### Database Screening and Sequence Analyses

Retroviral endogenous env gene sequences were searched by BLAST® on the human genome (GRCh38/hg38 Genome Reference Consortium Human Reference 38 (GCA_000001405.15), December 2013). First, all genomic sequences containing an ORF longer than 400 aa (from start to stop codons) were extracted from the hg38 human database using the GETORF program of the EMBOSS package (http://emboss.sourceforge.net/apps/cvs/emboss/apps/getorf.html) and translated into amino acid sequences. These amino acid sequences were then BLASTed against the SU-TM amino acid sequences of 42 retroviral envelope glycoproteins (from representative ERVs among which are known syncytins, and infectious retroviruses), using the BLASTP program of the National Center for Biotechnology Information (NCBI®; www.ncbi.nlm.nih.gov/BLAST). Positive envelope-containing ORFs were classified by multiple alignments of their amino acid sequences using the ClustalW protocol (www.ebi.ac.uk). ORFs consisting of highly repetitive sequences were discarded.

Maximum-likelihood phylogenetic trees were constructed with RaxML 7.3.2, with bootstrap percentages computed after 1,000 replicates using the GAMMA+GTR model for the rapid bootstrapping algorithm.

Sequences were analyzed using various platforms and softwares: UCSC browser of the Santa Cruz University of California (https://genome.ucsc.edu/); REPBASE (http://www.girinst.org/repbase/); REPEATMASKER (http://www.repeatmasker.org); DFAM of the University of Montana (http://www.dfam.org/); EMBOSS® softwares at the CBiB-Bordeaux, France (http://services.cbib.u-bordeaux.fr/galaxy/), prediction servers at (http://www.cbs.dtu.dk/services/) and (http://www.expasy.org/) and NEWCPGREPORT for CpG island characterization at (http://www.ebi.ac.uk/Tools/emboss/).

dN/dS ratios were obtained with the PAML program package, on the PAMLX graphical user interface (version 1.2). Coordinates of the selected HEMO ORF sequences are listed in Table 2 below. The gibbon, baboon, spider monkey and saki nucleotide HEMO ORF were PCR-amplified as indicated below, and the sequences deposited in GEN BANK®.

Syntenic loci were recovered for a representative number of species from the UCSC browser, on a 250 kb genomic region located between two genes conserved in all species, 5′ and 3′ to the HEMO locus, namely RASL11B and USP46. They were analyzed using the MULTIPIPMAKER alignment tool (http://pipmaker.bx.psu.edu/pipmaker/), with the human genome sequence as a reference. Coordinates of the selected sequences are listed in Table 3 below.

TABLE 2
List of genomic coordinates of the simian HEMO ORF sequences

| Species | Assembly | Coordinates |
|---|---|---|
| Human | GRCh38/hg38, 2013 | chr4: 52,743,829-52,745,520 |
| Chimpanzee | CSAC 2.1.4/panTro4 | chr4: 77,303,213-77,304,904 |
| Gorilla | gorGor4.1/gorGor4, 2014 | chr4: 76,069,071-76,070,762 |
| Orangutan | WUGSC 2.0.2/ponAbe2, 2007 | chr4: 67,458,017-67,459,708 |
| Gibbon | deposited | |
| Macaque | BCM Mmul_8.0.1/rheMac8, 2015 | chr5: 82,025,079-82,026,770 |
| Baboon | deposited | |
| AGM | Chlorocebus_sabeus 1.1/chlSab2, 2014 | chr7: 15,740,103-15,741,791 |
| Colobus angolensis palliatus | Cang.pa_1.0 | Scf473: 131,759-473,133,450 |
| Langur | deposited | |
| Marmoset | WUGSC 3.2/calJac3, 2009 | chr3: 140,763,785-140,765,366 |
| Rhinopithecus roxellana | isolate Xiao Hai Rrox_v1 | ENSRROG025365: 167,098-168,786 |
| Squirrel monkey | Broad/saiBol1, 2011 | JH378162: 9,809,510-9,811,090 |
| Spider monkey | deposited | |
| Saki monkey | deposited | |
| Cat | ICGSC/Felis_catus_8.0/felCat8, 2014 | chrB1: 164,136,405-164,138,141 |

TABLE 3
List of genomic coordinates of the 250 kb RAS-USP46 locus

| Species | Assembly | Coordinates |
|---|---|---|
| EUARCHONTOGLIRES | | |
| Human (Simian-Ape) | GRCh38/hg38, 2013 | chr4: 52,590,972-52,866,835 |
| Chimpanzee-Bonobo (Simian-Ape) | Max-Planck/panPan1, 2012 | JH650087: 949069-1220194 |
| Rhesus macaque (Simian-OWM) | BCM Mmul_8.0.1/rheMac8, 2015 | chr5: 81,899,847-82,185,019 |
| Marmoset (Simian-NWM) | WUGSC 3.2/calJac3, 2009 | chr3: 140669174-140925562 |
| Tarsier (Prosimian) | Tarsius_syrichta-2.0.1/tarSyr2, 2013 | KE926088v1: 194120-271011 |
| | | KE938719v1: 458231-525407 |
| Mouse Lemur (Prosimian) | Mouse lemur/micMur2, 2015 | KQ053245v1: 1118456-1287657 |
| Colugo (Dermoptera) | G_variegatus-3.0.2 | scaffold969: 20581-246600 |
| Mouse (Rodentia) | GRCm38/mm10, 2011 | chr5: 74000038-74199471 |
| Guinea Pig (Rodentia) | Broad/cavPor3, 2008 | scaffold_24: 23257653-23479169 |
| Rabbit (Lagomorpha) | Broad/oryCun2, 2009 | chrUn0056: 1207035-1383532 |
| LAURASIATHERIA | | |
| Hedgehog (Insectivora) | EriEur2.0/eriEur2, 2012 | JH835325: 6037893-6282794 |
| Cow (Ruminantia) | Bos_taurus_UMD_3.1.1/bosTau8, 2014 | chr6: 69950422-70183806 |
| Horse (Perissodactyla) | Broad/equCab2, 2007 | chr3: 79263527-79467858 |
| Dog (Carnivora) | Broad/CanFam3.1/canFam3, 2011 | chr13: 45379782-45583929 |
| Cat (Carnivora) | ICGSC/Felis_catus_8.0/felCat8, 2014 | chrB1: 164058766-164262324 |
| AFROTHERIA | | |
| Elephant (Proboscidae) | Broad/loxAfr3, 2009 | scaffold_38: 3843013-4119717 |
| Tenrec (Tenrecidae) | Broad/echTel2, 2012 | JH980315: 5379386-5641477 |
| XENARTHRA | | |
| Armadillo (Dasypodidae) | Baylor/dasNov3, 2011 | JH568349: 4112648-4401060 |
| MARSUPIAL | | |
| Opossum (Didelphimorphia) | Broad/monDom5, 2006 | chr5: 173087922-173327904 |

Cell Culture, 5-Aza-2′-deoxycytidine Treatment and Metalloprotease Inhibitors

Cells were maintained at 37° C., 5% CO2 in DULBECCO'S MODIFIED EAGLE MEDIUM for 293T (embryonic kidney), HeLa (cervix adenocarcinoma), CaCo-2 (colon adenocarcinoma), TE671 (rhabdomyosarcoma), SH-SY5Y (neuroblastoma) and HuH7 (hepatoma) human cells, in RPMI Media 1640, for JAR (choriocarcinoma), 2102Ep (teratocarcinoma) and NCCIT (teratocarcinoma) human cells, and in F-12K Medium for BeWo (choriocarcinoma), JEG-3 (choriocarcinoma) and NTera2D1 (teratocarcinoma) human cells. All media were supplemented with 10% heat-inactivated fetal calf serum (FCS), 100 U/ml penicillin, and 100 μg/ml streptomycin (all reagents are from LIFE TECHNOLOGY). iPSC were grown on irradiated MEFs at the GUSTAVE ROUSSY iPSC-platform. When reaching confluence, cells were serum-deprived for 36 hours and supernatant was harvested, filtered (0.22 μm Millipore filters) and concentrated 20-fold on AMICON Ultra 0.5 mL (MILLIPORE, 10K).

For treatment with 5-Aza-2′-deoxycytidine (5-Aza-dC; SIGMA-ALDRICH), 2×105 BeWo and 293T cells were plated in 6-well dishes. Doses ranging from 0.1 to 5 μM of 5-Aza-dC were then added to the culture for 3 days, with fresh medium each day. Cells were harvested for RNA extraction one day later.

For treatment with metalloprotease inhibitors, 5×105 293T cells were seeded in 6-well dishes with 2 mL/well of culture medium. One day after seeding, cells were transiently transfected using 1.5 μg phCMV-HEMO plasmid and 4.5 μL Lipofectamine LTX (THERMOFISHER) per well. One day post-transfection, cells were incubated with culture medium supplemented with the indicated concentrations of metalloprotease inhibitors (CALBIOCHEM): BATIMASTAT (0.1 to 10 μM), MARIMASTAT (0.1 to 10 μM) or GM6001 (1 to 50 μM). Medium with inhibitors was replenished for 2 other days, and supernatants were collected and filtered through 0.45 μm MILLIPORE filters one day later. Cells were harvested the same day for protein analysis.

### Luciferase Promoter Assay

For HEMO promoter activity assay, fragments of different sizes containing the TSS (+1) were PCR-amplified from human genomic DNA, and cloned in sense and antisense orientation, into the HindIII-NheI sites of the pGL3 Basic vector (PROMEGA) upstream of the luciferase reporter gene (757 bp fragment: from −290 to +472; 467 bp fragment: from +1 (TSS) to +472; 408 bp fragment: from +57 to +472; primers used are listed in Table 4 below, with (NNN) representing HindIII and NheI sites).

293T cells were seeded in 96-well dishes with 2×10⁴ cells per well. One day after seeding, cells were transfected with 100 ng DNA plasmid and 0.2 μL JETPRIME® (POLYPLUS TRANSFECTION; 850 boulevard Sébastien Brant; 67400 Illkirch; France). Two days post-transfection, culture medium was discarded and the activity of luciferase was detected using the PIERCE™ RENILLA-Firefly Luciferase Dual Assay Kit and the GLOMAX®-Multi+ Luminescence Apparatus (PROMEGA) following the manufacturer's instructions.

TABLE 4
list of primers

| Primer names | Primer sequences |
|---|---|
| qRT-PCR | |
| hemo-F1 | 5′-ACTATGGGCTCCCTTTCAAACT (SEQ ID NO: 77) |
| hemo-R1 | 5′-CATAGGAGGAAGTAGAGTGATT (SEQ ID NO: 78) |
| RPLP0-F | 5′-GGCGACCTGGAAGTCCAACTA (SEQ ID NO: 79) |
| RPLP0-R | 5′-CCATCAGCACCACAGCCTTC (SEQ ID NO: 80) |
| G6PD-F | 5′-TGCAGATGCTGTGTCTGG (SEQ ID NO: 81) |
| G6PD-R | 5′-CGTACTGGCCCAGGACC (SEQ ID NO: 82) |
| RACE experiments | |
| hemo-5′-RACE-R | 5′-CCTTGGGAGGTCCTAGTGCTAAGTGC (SEQ ID NO: 83) |
| hemo-3′-RACE-F | 5′-AAGCCACAGGAAGCTAGATTGAGATCAT (SEQ ID NO: 84) |
| hemo-R2 | 5′-GCTGTCTACTTCATCTGCTCAT (SEQ ID NO: 85) |
| hemo-R4 | 5′-CCGCAGACGTAGACAACGAA (SEQ ID NO: 86) |
| hemo-F4 | 5′-TTTCAAATAGGGCAATGAAGG (SEQ ID NO: 87) |
| panMars-5′-RACE-R | 5′-CATCTGTCCTCTGGAACATCGCCCAAG (SEQ ID NO: 88) |
| panMars-R2 | 5′-TCAGTTTCCATATTACCCACTT (SEQ ID NO: 89) |
| panMars-R3 | 5′-CAAGGAGTGAACTGAAGTGG (SEQ ID NO: 90) |
| panMars-R4 | 5′-ATTCGTCAGAACAACCCAATAG (SEQ ID NO: 91) |
| Bisulfite experiments | |
| Fragment I | I-F 5′-AGGTAGGTAGTGGATATAGGTG (SEQ ID NO: 92) |
| | I-R 5′-AAACCAAAAAACCAAAAAAA (SEQ ID NO: 93) |
| Fragment I Nested | I-F2 5′-GTAGTGGATATAGGTGGTT (SEQ ID NO: 94) |
| | I-R2 5′-AAACCAAAAAACCAAAAAAAAAAC (SEQ ID NO: 95) |
| Fragment II | II-F 5′-TTTTTTTTTTGGTTTTTTGG (SEQ ID NO: 96) |
| | II-R 5′-ATCTACCCTAAAAAACAAA (SEQ ID NO: 97) |
| Fragment II Nested | II-F2 5′-TTTTTTTTTTGGTTTTTTGG (SEQ ID NO: 98) |
| | II-R2 5′-AAAAAACAAAACRCAAACTTATTAC (SEQ ID NO: 99) |
| Amplification of | |
| genomic hemo in | |
| primate species | |
| hemoGe-F-Xho | 5′-ATACATCTCGAGCATTGTCTGGAGTTTGCTTGT (SEQ ID NO: 100) |
| hemoGe-R-Mlu | 5′-ATACATACGCGTGGGTAAGGGTTTACAGATCAG (SEQ ID NO: 101) |
| hemoNWM-R-Mlu | 5′-ATACATACGCGTACACCTTGGGAGGTCCTAGT (SEQ ID NO: 102) |
| Amplification of | |
| promoter fragments | |
| hemo-(−290)F | 5′-(NNN)GTCCTGCCCTCGTCCCGAAG (SEQ ID NO: 103) |
| hemo-(+1)F | 5′-(NNN)CACTTCAGTTCCCGCCGCGA (SEQ ID NO: 104) |
| hemo-(+57)F | 5′-(NNN)GCCAGTTTATCCCTCGGAGTT (SEQ ID NO: 105) |
| hemo-(472)R | 5′-(NNN)CCGCAGACGTAGACAACGAA (SEQ ID NO: 106) |
| Furin site mutation | |
| hemo-RTKR-F | 5′-CACCGCATAGACGCACCAAACGAGACACAGACA (SEQ ID NO: 107) |
| hemo-RTKR-R | 5′-TGTCTGTGTCTCGTTTGGTGCGTCTATGCGGTG (SEQ ID NO: 108) |

### Bisulfite Genomic Sequencing Analysis

Genomic DNA from 293T, BeWo, iPSC-NP24, and CaCo-2 cells were subjected to bisulfite treatment with the EpiTect Plus DNA Bisulfite Kit (QIAGEN). 2 DNA fragments of the promoter region were amplified via nested PCR (2 rounds of 35 cycles) with ACCUPRIME™ High Fidelity polymerase (INVITROGEN, @THERMO-FISCHER), on 50 to 150 ng bisulfite treated DNA, using specific primers listed in Table 4 above. PCR products were then cloned into pGEMT-Easy vector (PROM EGA) and a minimum of 10 clones were selected for sequencing.

Expression Vectors for the HEMO ORF from Human and Simians and Ex Vivo Assays

The HEMO ORF from human and selected simians (FIGS. 11A and 11B) were PCR-amplified from the corresponding genomic DNAs using the PHUSION DNA Polymerase (THERMO SCIENTIFIC) with a unique forward primer due to high conservation 5′ to the ATG codon (hemoGe-F-Xho), and one of the two reverse primers (hemoGe-R-Mlu or a specific NWM monkey hemoNWM-R-Mlu primer), see Table 4 above. PCR products were directly sequenced (BIGDYE TERMINATOR v3.1, THERMOFISCHER). The amplified HEMO gene fragments were then cloned into the Xho I and Mlu I sites of the phCMV-G expression vector (GENBANK® accession AJ318514), for transfection experiments. Premature stop codon HEMO mutants (FIG. 4) were constructed by inserting a TGA-stop codon in a reverse primer used to PCR-amplify the indicated fragments from phCMV-HEMO, and recloning as above. Substitution of the CTQG sequence by the consensus furin site RTKR (as in the NWM HEMO genes) was performed by site-directed mutagenesis with multiple PCR reactions.

HEMO protein production and release were assayed using 5×10⁵ 293 T cells transfected with 1.5 μg of phCMV-HEMO plasmid and 7.5 μl Fugene 6 (PROM EGA) in 6-well dishes. Cell media were replaced 12 h post-transfection by serum-free media. Forty-eight hours post-transfection, supernatant and cells were collected. Supernatants were filtered (0.45 μm MILLIPORE filters) and stored at −80° C. For cell lysates, samples were solubilized in RIPA buffer (150 mM NaCl, 25 mM Tris HCl pH 7.6, 0.1% SDS and 1% sodium deoxycholate, THERMO SCIENTIFIC) with 1×-Protease and Phosphatase Inhibitor Cocktail (THERMO SCIENTIFIC), centrifuged (14,000 g for 20 min to eliminate debris), and stored at −80° C. before testing.

### Immunofluorescence and Immunohistochemistry Assays

For HEMO immunofluorescence assays, Hela cells were grown on glass coverslips, and transiently transfected with the phCMV-HEMO expression vector or a control empty vector (500 ng) and 1.5 μL Lipofectamine LTX (THERMOFISHER) per well of 12-well dishes. Forty-eight hours post-transfection, cells were fixed in 4% paraformaldehyde, permeabilized or not with 0.2% TRITON X100, and stained with the mouse anti-HEMO polyclonal antibody (see above) and an ALEXA Fluor 488-conjugated anti-mouse secondary antibody (MOLECULAR PROBES). Nuclei were stained in blue with DAPI (SIGMA-ALDRICH). Observations were made under a LEICA TCS SP8 MP confocal microscope.

For immunohistochemistry assays, freshly collected placental tissues were fixed in 4% paraformaldehyde and embedded in paraffin. Sections (4 μm) were stained with hematoxylin eosin and safran. Paraffin sections were processed for heat-induced antigen retrieval (Tris EDTA pH 9, ABCAM) and incubated overnight with the monoclonal mouse anti-HEMO (2F7) antibody (1/10 dilution) or a control IgG2a isotype. Staining was visualized by using the peroxidase/diaminobenzidine Mouse PowerVision kit (IMMUNOVISION TECHNOLOGIES).

### Western Blot Analyses, Wheat Germ Agglutinin Purification and Peptide N-Glycosidase F Treatment

Samples, cell supernatants or cell lysates were analyzed by SDS/PAGE on gradient precast gels (NuPAGE NOVEX 4-12% Bis-Tris gels, LIFE TECHNOLOGIES), and transfer onto nitrocellulose membranes using a semi-dry transfer system. After blocking in PBS containing 0.1% Tween-20 and 5% nonfat milk, membranes were incubated overnight at 4° C. with primary antibodies (anti-HEMO mouse polyclonal antibody 1/5000, anti-CGB/hCG-beta rabbit polyclonal antibody (ABGENT) 1/100000, anti-γ-tubulin mouse monoclonal antibody (SIGMA-ALDRICH) 1/1000), washed 3-times and then incubated with species-appropriate horseradish peroxidase (HRP)-conjugated secondary antibodies for 45 min at RT. Proteins were detected by using an enhanced chemiluminescence system (ECL, PIERCE).

When specified, glycoproteins were first extracted from placental tissue or sera, using the lectin wheat germ agglutinin (WGA) kit (THERMO SCIENTIFIC). Six hundred microliters of whole protein extracts were prepared according to the manufacturers' guidelines and eluted in 200 μl elution buffer. When specified, samples were treated with peptide N-glycosidase F (PNGase F; NEB BIOLABS) before SDS-PAGE.

### Mass Spectrometry Characterization of the N- and C-Termini of the HEMO Protein

To get sufficient amounts of HEMO protein for MS characterization, 293T cells (4 10-cm dishes with 3×10⁶ cells/dish) were transfected with the phCMV-HEMO expression vector (from human), in DMEM-FCS medium (10 μg per plate). Medium was replaced by serum-free DMEM 2 days later, and supernatants recovered after 2 more days. Total secreted proteins were concentrated about 60-fold using VIVASPIN 20 (SARTORIUS, 30,000 MWCO PES). Glycoproteins from the concentrated extract were recovered using the WGA-kit, eluted in 200 μL, and loaded on a 4-12% NuPAGE gel. The 80 kDa part of the acrylamide gel was excised and proteins eluted in a dialysis bag electrophoretically. Proteins were again concentrated using AMICON Ultra Centrifugal Filters (ULTRACEL-50K), treated with PNGase and re-loaded on a 4-12% NuPAGE gel for an additional purification step. The main band (seen upon Coomassie Blue staining and corresponding to the shed 48 kDa HEMO protein) was excised and subjected independently to different enzymatic digestions (Trypsin, Chymotrypsin). The shed HEMO protein associated fragments were characterized by the IMAGIF platform of Gif-sur-Yvette (France), by nanoLC-MS/MS analyses with a Triple-TOF 4600 mass spectrometer (AB Sciex, Framingham, Mass., USA), thus allowing the determination of the N- and C-termini of the protein.

### RNA, Real-Time RT-PCR and RACE Experiments

Total RNAs from human tissues and cells were either purchased from ZYAGEN (San Diego), or isolated using the RNAeasy Isolation Kit (QIAGEN) according to the manufacturer's instructions, and treated with Dnase I (AMBION). Reverse transcription was performed with 1 μg of RNA using the MLV reverse-transcriptase (APPLIED BIOSYSTEMS). Real-time quantitative PCR was carried out with 5 μl of diluted (1:20) cDNA in a final volume of 25 μl by using SYBR green PCR master mix (QIAGEN). qPCR was carried out with an ABI Prism 7000 sequence detection system, using primers listed in Table 4 above. Transcript levels were normalized relative to the amount of a housekeeping gene (RPLPO or G6PD) mRNA. Samples were assayed in duplicate.

5′-RACE and 3′-RACE were performed with 100 ng of DNase-treated RNA using the SMARter RACE cDNA Amplification Kit (CLONTECH), and the primers listed in Table 4 above.

### RNA-Seq Data Mining

RNA-seq raw data were downloaded from NCBI® Sequence Read Archive (SRA) with Accession numbers: SRP011546 (GSE36552), ERP003613 (PRJEB4337) and SRP042153 (GSE57866). RNA-seq raw data were aligned with TOPHAT2 (v2.0.14) to a custom gene database of interest, including some retroviral envelope and housekeeping genes, with the following parameters: “--read-mismatches 0 -g 1 --no-coverage-search”. Uniquely mapped reads were selected using SAMtools (v0.1.19) for further analysis. Only hits with exact matches were counted in order to avoid detection of other analogous ERV genes. Read counts were normalized by the length of the gene (after merging in kilobases) and by the read counts of two housekeeping genes (RPLPO and RPS6) and log transformed. Specific transcripts of the gene (absence of read counts in intronic and flanking sequences, and presence of split RNA-seq reads corresponding to specific splice junctions) were also verified by blast on the NCBI®-Trace Archive Nucleotide BLAST® platform. For each gene of interest, read counts were verified to be equally distributed over the coding sequence, on the Integrative Genomics Viewer visualization tool (http://software.broadinstitute.org/software/igv).

### Microarray Data Mining

To get insight into the expression profile of the HEMO gene in normal and tumoral human tissues, an in silico analysis of microarray data was performed, using the dataset E-MTAB-62 elaborated in Lukk et al. 2010 (https://www.ebi.ac.uk/arrayexpress/experiments/E-MTAB-62/files/), which includes 1033 samples from normal tissues and 2315 neoplasm samples, obtained from various AE and Gene Expression Omnibus (GEO) studies. The dataset E-MTAB-62 was downloaded as processed expression data. Statistical significance was assessed using Wilcoxon's rank sum test. For larger panel analyses, additional ovarian cancer datasets (AE E-GEOD-63885, E-GEOD-30311, E-GEOD-54809, E-GEOD-6008, E-GEOD-14764, from https://www.ebi.ac.uk/arrayexpress) were pre-processed using the “expresso” function of the affy package (v1.48.0) (Gautier et al. 2004), with the following parameters: robust multiarray average (RMA) for background correction, keeping only the perfect match (PM) probes (“pmonly”), and quantile normalization. Both “medianpolish” and “avgdiff” were applied as summarization methods, in order to have normalized values in both log 2-transformed values and probeset intensities log 2-transformed values. After data pre-processing, the expression values of the HEMO gene was extracted and plotted with R 3.2.3 (www.r-project.org). The ovarian cancer datasets were merged using the inSilicoMerging R package (Taminau et al. 2012) (version 1.14), applying COMBAT as batch effect correction method.

### Results

### Identification of HEMO, an HERV Env Gene Encoding a Full-Length Protein

The most recent human genome sequence release (GRCh38 Genome Reference Consortium Human reference 38, December 2013) was screened for the presence of genes encoding full-length ERV Env proteins by a BLAST search for ORFs (from the Met start codon to the stop codon) >400 aa, using a selected series of 42 Env sequences representative of both infectious retrovirus and ERV families, including all the previously identified syncytins (Methods). It yielded 45 Env-encoding ORF, which could be, for all except one, grouped by clustalW alignments into already known HERV Env families (among which 24 Env-encoding ORFs for HERV-K, and 20 Env-encoding ORFs belonging to the set of 12 previously described HERV Envs, see Table 5 below).

TABLE 5
Human Envelope protein coding sequences genomic coordinates

| | Name | Length | Coordinates |
|---|---|---|---|
| ENV GAMMA-type | EnvW | 538aa | chr7: 92468768-92470381 (REVERSE SENSE)) |
| | EnvW-like | 475aa | (chrX: 107052509-107053933 (REVERSE SENSE)) |
| | EnvW-like | 472aa | (chr20: 55351277-55352692) |
| | EnvW-like | 468aa | (chr4: 72926505-72927908 (REVERSE SENSE)) |
| | EnvFRD | 538aa | (chr6: 11103697-11105310 (REVERSE SENSE)) |
| | EnvERV3 | 604aa | (chr7: 64991215-64993026 (REVERSE SENSE)) |
| | EnvERV3-like | 406aa | (chrX: 52569735-52570952 (REVERSE SENSE)) |
| | EnvE | 428aa | (chr19: 20748111-20749394 (REVERSE SENSE)) |
| | EnvV1 | 477aa | (chr19: 53014091-53015521) |
| | EnvV2 | 535aa | (chr19: 53049252-53050856) |
| | EnvH1 | 584aa | (chr2: 165708193-165709944 (REVERSE SENSE)) |
| | EnvH2 | 563aa | (chr3: 166823237-166824925 (REVERSE SENSE)) |
| | EnvH3 | 555aa | (chr2: 154872220-154873884) |
| | EnvH-like | 474aa | (chrX: 72228564-72229985) |
| | EnvPb | 665aa | (chr14: 92622888-92624882 (REVERSE SENSE)) |
| | EnvRb | 514aa | (chr3: 16770303-16771844) |
| | EnvFc1 | 584aa | (chrX: 97847263-97849014) |
| | EnvFc2 | 527aa | (chr7: 153409531-153411111 (REVERSE SENSE)) |
| | EnvT | 626aa | (chr19: 20369432-20371309) |
| | EnvT-like | 427aa | (chr14: 106197668-106198948 (REVERSE SENSE)) |
| | EnvHEMO | 563aa | (chr4: 52743832-52745520 (REVERSE SENSE)) |
| ENV BETA-type | Env-K-like | 412aa | (chr16: 10418516-10419751 (REVERSE SENSE)) |
| | Env-K-like | 439aa | (chr1: 242457592-242458908) |
| | Env-K-like | 475aa | (chr5: 34462280-34463704 (REVERSE SENSE)) |
| | Env-K-like | 482aa | (chr16: 2661368-2662813 (REVERSE SENSE)) |
| | Env-K-like | 487aa | (chr11: 118722384-118723844 (REVERSE SENSE)) |
| | Env-K-like | 550aa | (chr16: 34413088-34414737 (REVERSE SENSE)) |
| | Env-K-like | 550aa | (chr16: 34997093-34998742) |
| | Env-K-like | 560aa | (chr1: 160697328-160699007) |
| | Env-K-like | 588aa | (chr1: 75380770-75382533) |
| | Env-K-like | 597aa | (chr3: 113025711-113027501 (REVERSE SENSE)) |
| | Env-K-like | 658aa | (chr12: 105311338-105313311) |
| | Env-K-like | 661aa | (chr11: 101701507-101703489) |
| | Env-K-like | 687aa | (chr2: 129962883-129964943 (REVERSE SENSE)) |
| | Env-K-like | 698aa | (chr12: 58328384-58330477 (REVERSE SENSE)) |
| | Env-K-like | 698aa | (chr6: 77717862-77719955 (REVERSE SENSE)) |
| | Env-K-like | 699aa | (chr7: 4583351-4585447 (REVERSE SENSE)) |
| | Env-K-like | 699aa | (chr7: 4591855-4593951 (REVERSE SENSE)) |
| | Env-K-like | 699aa | (chr8: 7498800-7500896 (REVERSE SENSE)) |
| | Env-K-like | 699aa | (chr19: 27638542-27640638 (REVERSE SENSE)) |
| | Env-K-like | 738aa | (chr3: 101696851-101699064) |
| | Env-K-like | 885aa | (chr3: 185564943-185567597 (REVERSE SENSE)) |
| | Env-K-like | 930aa | (chr5: 156659966-156662755 (REVERSE SENSE)) |
| | Env-K-like | 1171aa | (chr22: 18943415-18946927) |
| | Env-K-like | 1375aa | (chr1: 155627591-155631715 (REVERSE SENSE)) |

Yet, an unrelated env gene (HEMO, for Human Endogenous MER34 ORF) can be identified (see FIG. 1) with a full-length 563 amino acid ORF displaying some—but not all—of the characteristic features of a bona fide retroviral Env protein, namely a signal peptide, a CWLC motif in the putative SU subunit and a C-X6-CC motif in the putative TM subunit, a 23 aa hydrophobic domain located in the TM transmembrane domain, and an ISD domain. Noteworthily, the putative HEMO protein lacks a clearly identified furin cleavage site (CTQG instead of the canonical R/K-X-R/K-R), as well as an adjacent hydrophobic fusion peptide (FIG. 1B). The HEMO sequence was incorporated into the Env phylogenetic tree shown in FIG. 1D, containing the 42 retroviral envelope aa sequences used for the genomic screen. The figure shows that the sequence most closely related to the HEMO protein is Env-panMars, encoded by a conserved, ancestrally captured retroviral env gene found in all Marsupials, and which has a premature stop codon upstream of the transmembrane domain (FIG. 12A-E).

Finally, BLAST analysis of the human genome indicates that the HEMO gene is part of a very old degenerate multigenic family known as MER34 (for MEdium Reiteration frequency family 34, first described in Toth and Jurka 1994). In this family, a MER34-int consensus sequence with a Gag-Pro-Pol-Env retroviral structure and LTR-MER34 sequences have been described and reported in RepBase (Jurka et al. 2005). Genomic blast with the MER34-int consensus sequence could not detect any full length putative ORFs for the gag or pol genes. Among the env sequences of the MER34 family scattered in the human genome (20 copies with >200 bp homology identified by blast, cf. Table 6 below), HEMO is clearly an outlier (1692 bp/563 aa), with all the other sequences containing numerous stop codons, Alu or LINE insertions, and no ORF longer than 147 aa.

TABLE 6
MER34-related env sequences in the human genome

| | | max ORF |
|---|---|---|
| Chromosome | extracted sequences^{a} | bp/aa |
| 2 | 162084066-162086565 (rev) | 195/64 |
| 2 | 110061551-110064050 | 201/66 |
| 2 | 110307369-110309868 (rev) | 195/64 |
| 2 | 208368352-208370851 | 279/93 |
| 3 | 83422568-83425067 (rev) | 213/70 |
| 4 HEMO | 52743421-52745920 (rev) | 1692/563 |
| 6 | 24704890-24713439 (rev) | 168/55 |
| 7 | 123922822-123925321 (rev) | 156/51 |
| 8 | 59785528-59788027 | 297/98 |
| 8 | 88712235-88714734 | 339/112 |
| 9 | 11114167-11116166 | 243/80 |
| 11 | 102483445-102485944 | 249/82 |
| 12 | 54733146-54735645 | 189/62 |
| 14 | 70462086-70464585 | 150/49 |
| 14 | 70237764-70240263 (rev) | 228/75 |
| 14 | 53023713-53026212 | 369/122 |
| 15 | 5078981-5081480 (rev) | 387/128 |
| 22 | 23932542-23935041 | 444/147 |
| 22 | 23938277-23940776 (rev) | 324/107 |
| X | 43460042-43462541 | 111/36 |

^{a}correspond to genomic sequences sorted out by BLAST with the MER34-env consensus (Repbase MER34-int, bp 6555-8207), and with >200 bp homology

### The HEMO Gene Locus and Transcription Profile

The HEMO gene is located on chromosome 4q12, between the RASL11B and USP46 genes, at about 120 kb from each gene (see also FIG. 10). Close examination of the HEMO env gene locus (10 kb), by BLAST comparison with the RepBase MER34-int consensus (Jurka et al. 2005), reveals only remnants of the retroviral pol gene in a complex scrambled structure (see FIG. 2A) with part of it being in reverse orientation and further disrupted by numerous Alu (SINE) insertions. The locus organization indicates low selection pressure for the proviral non-env genes, as often observed in the previously characterized loci harboring captured envs.

A quantitative RT-PCR (RT-qPCR) analysis using primers within the identified ORF and RNAs from a panel of human tissues and cell lines (FIG. 2B) shows that HEMO is expressed at a high level in the placenta. It is also significantly expressed in the kidney but at a lower level. In cell lines, expression of the HEMO gene looks heterogeneous, except for its systematic expression in stem cells (ESC and iPSC). Quite unexpectedly there is an absence of detectable transcripts in several placental choriocarcinoma cell lines (BeWo, JAR, JEG-3), as well as in a series of embryonal carcinoma (NT2D1, 2102Ep, NCCIT) and tumor cell lines (but see the CaCo-2 colon adenocarcinoma).

The structure of the HEMO env transcripts was determined by RACE-PCR analysis of Env-encoding transcripts from the placenta. It allowed the identification of multiply-spliced transcripts, with the intron boundaries corresponding to donor/acceptor splice sites predicted from the genomic sequence and, as classically observed for retroviral env genes, a functional acceptor site located close to the env ATG start site. Interestingly, the transcript 3′-end falls within an identifiable MER34 LTR, as expected for a retroviral transcript. Yet, the transcription start site, located approximately 5 kb 5′ to the env gene, does not correspond to any identifiable LTR structure. Rather, the sequence associated with the transcript start site is located in a CpG-rich domain (FIGS. 2A and 2C), and most probably corresponds to a cellular promoter unrelated to any retroviral element. The transcript 5′-end, i.e. tc|ACTTC, falls within a canonical RNA Polymerase II Core Promoter Initiator Motif (yy|ANWYY).

The CpG-rich start site containing region (CpG island, reviewed in Deaton and Bird 2011) was studied further for its promoter activity by ex vivo transfection assays, using luciferase reporter genes. As illustrated in FIG. 2D, a 760 bp fragment including the identified start site acts as a strong promoter in this assay (>500 fold compared to none). Lower expression is observed (10 to 50 fold compared to none) in partial deletion mutants and, as expected for a CpG promoter, when placed in antisense orientation.

DNA methylation patterns of sequences surrounding the transcription start site within the identified CpG island were analyzed by bisulfite treatment. As shown in FIG. 2E, the majority of the CpGs are methylated in the HEMO-negative cell lines (293T, BeWo), whereas they are unmethylated in HEMO-expressing cell lines (iPSC and CaCo-2). To get further insight into this dependence of the promoter activity on the CpG island methylation pattern, 5-Aza-2′-deoxycytidine (5-Aza-dC) treatment was performed on BeWo and 293T cells at doses ranging from 0.1 to 5 μM (FIG. 2F). Transcripts were detectable by qRT-PCR after a 3-day treatment, at low dose for BeWo cells (0.1 μM), and higher dose for 293T cells (5 μM). Of note, the high transcript level of the HEMO gene in CaCo-2 cells was not further amplified by a similar 5-Aza-dC treatment. Altogether these results indicate that HEMO expression is sensitive to the methylation status of the CpG promoter.

### HEMO Protein Synthesis and Structure: Specific Shedding

The capacity of the identified gene to produce an envelope protein was tested by introduction of the env ORF into a CMV promoter-driven expression vector, and ex vivo transient transfection assays. Polyclonal and monoclonal antibodies were raised by immunization of mice with a recombinant protein corresponding to a 163 aa fragment of the putative SU moiety of the protein (see Methods). As illustrated by the immunofluorescence assay shown in FIG. 3—upper panel, using the anti-HEMO antibodies, a strong labeling can be observed upon permeabilization of the transfected cells (and not of control cells, transfected with an empty vector). Furthermore, HEMO proteins can be detected at the cell surface, as evidenced by the specific immunofluorescence labelling of the cell membrane of non-permeabilized transfected HeLa cells, in the successive confocal images shown in FIG. 3—lower panel, consistent with HEMO being a retroviral env gene.

As illustrated in the Western blot of a whole cell lysate (FIG. 4A, lane 3), transfection with the above HEMO expression vector yielded a strong band with an apparent molecular weight >80 kDa, much larger than expected for the HEMO full-length SU-TM protein (theoretical MW 61 kDa), but consistent with its glycosylation—as expected for a retroviral protein. Indeed, treatment of the cell extract with Peptide N-Glycosidase F (PNGase F)—to de-glycosylate proteins—resolved the >80 kDa band into two bands, of lower molecular weight (lane 4): a major band of approximately 58 kDa, and a fainter one of 48 kDa. The major band most probably corresponds to the full-length SU-TM protein (expected size 61 kDa), whereas the lower band has a size inconsistent with that of the sole SU subunit (expected size 37 kDa)—that could be potentially generated by SU-TM cleavage at a furin site (although not canonical in human HEMO (CTQG instead of RXKR, see below).

Analysis of the cell supernatants provided an unexpected answer as to the origin of the 48 kDa protein. Indeed, this 48 kDa protein turns out to be the major form in the cell supernatant (see FIG. 4A, lane 6, with PNGase F-treatment of the supernatant), whereas the larger 58 kDa band observed in the whole cell extract (FIG. 4A, lane 4, with similar PNGase F-treatment) is almost undetectable, as expected for a cell membrane-attached full-length Env protein (FIG. 4A, lane 6). This secreted 48 kDa protein is glycosylated, being observed at a much higher molecular weight in the cell supernatant without PNGase-F treatment (FIG. 4A, lane 5). Altogether, these data strongly suggest that the HEMO protein, which is a transmembrane protein exported at the cell surface, can nevertheless be quantitatively released—shed—in the supernatant, in the form of a protein whose MW is larger than that of the SU alone. This property, unexpected for a retroviral Env protein, is indeed not observed using the same protocols and expression vectors for syncytin-1 (HERV env-W) used as a negative control (FIG. 4A lanes 1, 2).

To go further into the characterization of this shed, soluble protein, we purified it from the supernatant of transfected 293T cells (see Method) and characterized its sequence by using Mass Spectrometry (MS) for the determination of both its N- and C-terminus. As illustrated in FIG. 4B, which provides the HEMO protein sequence coverage by MS analysis of trypsin- or chymotrypsin-generated peptides, it turns out that the shed protein is truncated at its C-terminus, mainly at a position located in the ISD domain, with two C-terminal sites identified with a different abundance (namely, Q432 and R433 at a 4 to 1 ratio). At the N-terminus, the HEMO protein begins at position 27, i.e. 2 aa after the predicted signal peptide cleavage site (using SignalP 4.1 Server software, http://www.cbs.dtu.dk/services/SignalP/). To further ascertain the MS size determination of the shed HEMO protein, several mutants were constructed by inserting stop codons at the indicated positions (marked in FIG. 4B, C with asterisks (*): 433R-stop, 472P-stop and 489S-stop) or by introducing a consensus furin site RTKR at the expected position (human furin+ construct, H-fur+). Western blot analysis of the supernatant of the HEMO mutant transfected cells then clearly showed that the wild-type deglycosylated shed HEMO protein migrates as the R433-stop mutant. In addition, and as expected, the H-fur+ mutant displays a smaller 37 kDa band, consistent with the size expected for the deglycosylated SU subunit. Of note, the 472 and 489 stop mutants, although they still contain the shedding site sequence (aa 432/433) but not the transmembrane domain (aa 490 to 512), are simply secreted (and not further processed as the wild-type HEMO protein), suggesting that anchoring the env protein at the cell surface is required for the shedding process.

To determine if the shed form of the HEMO protein could be observed under in vivo conditions, placental tissues (which show high transcription levels for the HEMO gene, FIG. 2B) were recovered from first trimester legal abortions, together with the local placental blood (which bathes the placental villi and can be analyzed in parallel), and proteins were extracted and deglycosylated for Western blot analyses. As shown in FIG. 4A, lane 7, the small 48 kDa band (and a very faint SU-TM 58 kDa band) can be detected in the placental tissue extract. The 48 kDa band is also detected in the placental blood, most probably corresponding to the protein secreted by the placenta. Mass Spectrometry analysis (as above) of the 48 kDa protein in the corresponding gel bands confirmed the relevance of the immunological detection.

The release of a processed HEMO protein is reminiscent of what has been observed for the viral envelope protein of a completely unrelated virus, i.e. the Ebola filovirus, for which it has been further demonstrated that cleavage was mediated by a cell-associated ADAM protein (Dolnik et al. 2004). Accordingly, we tested whether chemical inhibitors of metalloproteinases (including the ADAM and MMP proteins, (Dolnik et al. 2004; Okazaki et al. 2012; Weber and Saftig 2012) had any effect on HEMO shedding in 293T transfected cells. As illustrated in FIG. 5, the broad range ADAM and MMP inhibitors BATIMASTAT and MARIMASTAT, and the MMP inhibitor GM6001 clearly inhibited HEMO release in the supernatant, to various extents and in a dose-dependent manner, with visible accumulation of the non-secreted form in the cell lysates. These experiments suggest that, in vivo, HEMO shedding could be driven by one or several metalloproteinases, known to be present notably in placental cells.

### HEMO Expression In Vivo: HEMO Release in the Blood Circulation of Pregnant Women

The combined results of the RT-qPCRs on the panel of human tissues shown in FIG. 2B and of the shedding of the protein shown in FIG. 4 led us to hypothesize that HEMO could be detected in the blood circulation, especially in pregnant women. Sera were therefore collected and assayed for the presence of shed HEMO by Western blotting. Sera were treated with wheat germ agglutinin (WGA) to isolate glycosylated proteins, which were then deglycosylated. As illustrated in FIG. 6 lower panel, the hCG-beta protein, which is a well-known early biomarker of pregnancy (Cole 2009), shows undetectable levels in the peripheral blood of men and non-pregnant women (lanes 2, 3), whereas a very high level is observed for women on the first trimester of pregnancy (20 kDa band, lanes 4 to 6), with a decrease at later stages (lanes 7 to 12). Remarkably, the de-glycosylated shed HEMO form (48 kDa, previously identified in the placental blood, FIG. 4A lane 8 and FIG. 6 upper panel, lane 1) can also be detected in the peripheral blood of pregnant women, beginning at a faint level in first trimester pregnancies (FIG. 6 upper panel, lanes 4-12). As pregnancy proceeds, the level of HEMO protein increases very significantly, consistent with the large increase in placental mass during pregnancy. HEMO concentration at the peak can be estimated to be in the 1-10 nM range (by comparative Western blot analysis of serial dilutions of a purified recombinant shed HEMO protein), i.e., is about 1 to 2 logs below that for hCG at the peak (T1) and, for further comparison, about the same as that for alpha-fetoprotein in the blood of pregnant women at the peak (T2). Of note, a faint level of shed HEMO protein can also be observed in men and non-pregnant women blood (FIG. 6, upper panel, lane 2 and 3), consistent with its non-negligible expression in other organs such as the kidney (see RTqPCR results in FIG. 2B).

As illustrated by FIG. 6, bands observed at both higher and lower MW might correspond to minor alternatively processed/shed forms of the HEMO protein (i.e., other than the 27-432/433 fragment; cf. FIG. 1C for the computation of the aa positions; SEQ ID NO: 9; SEQ ID NO: 10). These alternatively processed/shed forms include fragments, which extend from aa position 27 (first aa after signal peptide) up to, and including, an aa position chosen from among positions 450-480 and 380-420 (SEQ ID NOs: 35-55 and 13-23). These other HEMO soluble fragments correspond to cleavage sites n° 2 and n° 3 in FIG. 13 (cleavage site n° 1: aa positions 27 up to 432-433 (main cleavage site); cleavage site n° 2=aa positions 27 up to 450-480; cleavage site no 3=aa positions 27 up to 380-420).

### Identification of HEMO-Producing Cells in the Placenta

The human placenta is of the hemochorial type and is characterized by the presence of fetal villi in direct contact with—and bathed by—the maternal placental blood (FIG. 7A). These villi arise from the chorionic membrane—of fetal origin—and have an inner mononucleated cytotrophoblast layer (CT) underlying the surface syncytial layer, the syncytiotrophoblast (ST) (reviewed in Bischof et al. 2005; Maltepe and Fisher 2015). The placenta invades the maternal uterine part, with anchoring villi characterised by invasive extravillous trophoblasts (EVT).

To localize precisely HEMO expression in the placenta, immunohistochemistry experiments were then performed on sections of first trimester placental tissues from abortion cases. As illustrated in FIGS. 7B and 7C, specific staining was obtained with the monoclonal anti-HEMO antibody—and not with a control isotype as shown in panel B (4× magnification). In the four enlargements (60× magnification) shown in panel C—corresponding to the boxed placental villi and chorionic membrane of panel B—, strong staining is observed in the trophoblast cells, including the villous cytotrophoblasts (CT), the extravillous cytotrophoblasts (EVT) and the chorionic membrane trophoblasts, suggesting that HEMO is indeed produced by these cells. More diffuse staining is observed in the syncytiotrophoblast layer (ST), which is generated by CT fusion and is involved in the exchanges between fetal and maternal blood.

Altogether, the immunohistochemical analyses of the placenta carried out with the above anti-HEMO antibody show strong labeling essentially at the trophoblast level, and are consistent with the observed shedding of HEMO in the mother's blood (FIG. 6).

### Profile of HEMO Expression in Development

To get insight into the possible involvement of HEMO in embryonic development, we further analyzed by data mining, a series of human RNA-seq experiments deposited at the SRA-NCBI platform, corresponding to different stages of development (Yan et al. 2013; Xue et al. 2013; Friedli et al. 2014; Uhlén et al. 2015). Extraction of the expression profiles of a set of human genes was performed and the results are illustrated in FIG. 8 A-C for the HEMO, the syncytin 1 (Env-W), and the syncytin 2 (Env-FRD) env genes, as well as for specific genes expressed either in the placenta (GCM1) or in stem cells (OCT4/POU5F1). For each gene of interest, read counts were verified to be equally distributed over the coding sequence (see Methods). FIG. 8A clearly shows that HEMO has a wide expression profile, being expressed early in embryonic development, starting at the 8-cell stage up to the late blastocyst stage and being permanently expressed in the derived embryonic stem cells, from passage 0 up to passage 10. The HEMO gene RNA-seq expression profile found in stem cells confirms the RT-qPCR results shown in FIG. 2B and is clearly different from what is observed for the two human syncytin genes: Env-W which is expressed very early in development is completely down-regulated in the human stem cells, and Env-FRD remains almost undetectable. All three env genes (together with the placental GCM1 specific gene) are found in the RNA-seq samples of placental tissues (FIG. 8B), as expected. Finally, RNA-seq expression of HEMO was analyzed in the reprogramming experiments of differentiated somatic cells into iPSCs as described in Friedli et al. 2014, and hits reported in FIG. 8C highlight the specific reprogramming of the HEMO gene—not observed with Env-W and Env-FRD—which parallels the expected profile of expression of the OCT4/POU5F1 transcription factor. Of note, as illustrated in FIG. 8 D at the protein level, we could verify by Western blot analysis of iPSC in culture, that the HEMO gene expression unraveled above also results in the shedding of HEMO proteins, with a 48 kDa band detected in the iPSC supernatants.

Conclusively, the HEMO gene displays a specific pattern of expression—that includes ES cells—a feature possibly linked to the “capture” of a specific CpG-rich promoter of non-LTR origin, with the bona fide production of HEMO in the form of a soluble protein from at least trophoblast and stem cells.

### HEMO Expression in Tumors

To get insight into the possible expression of the HEMO gene in human tumors, we performed an in silico analysis of microarray data, using the dataset E-MTAB-62 elaborated in Lukk et al. 2010, which includes 1033 samples from normal tissues and 2315 from neoplasm tissues, obtained from various ArrayExpress (AE) and Gene Expression Omnibus (GEO) studies. In normal tissues, as expected from the RT-qPCR analysis in FIG. 2B, significant levels of expression were essentially observed in placental tissues, and to a limited extent in the kidney (FIG. 9A). In several tumors, as illustrated in FIG. 9B, heterogeneity was detected among samples from the same organ (represented by the outliers plotted as black dots), with in some cases evidence for high level expression of the HEMO gene: for instance in germline, liver, lung or breast tumors, with the most salient heterogeneity being observed for ovary tumors. In the latter case, further search for annotation data related to various histological types of ovarian carcinoma (Cho and Shih 2009; Kurman and Shih 2016), led to correlate the highest values with specific tumor histotypes, mainly Clear Cell Carcinoma.

To enlarge this data set, ovary tumor samples from 5 other GEO databases were collected and further normalized (see Methods) together with E-MTAB-62, giving a total of 479 tumor samples. As shown in FIG. 9C, higher expression values of the gene are observed for Clear Cell Carcinomas (60 samples) and, to a lesser extent, Endometrioid Cancer samples (96 samples). No clear-cut upregulation of the HEMO gene is observed in Serous Cancer histotype (289 samples, albeit with some heterogenity) and in the Mucinus histotype (34 samples).

In agreement with these transcription data, immunohistochemistry analyses of normal versus Clear Cell Carcinoma ovarian tissues, using the anti-HEMO monoclonal antibody, disclose a highly specific staining of the tumoral clear cells, as compared to the control isotype staining (FIG. 9D).

### HEMO Insertion Date and Conservation Across Mammalian Genomes

A strong hint for a physiological role of a captured gene is its conservation in evolution and the nature of the selection to which it is subjected. Accordingly, we performed an extensive search for the HEMO gene in eutherian mammals, both by in silico screening and by PCR-cloning and sequencing, and further extended it to marsupials (the phylogenetic tree in FIG. 1 shows homology of HEMO with env-panMars and see below). These analyses also aimed at the determination of the HEMO date of insertion into the genome of a mammalian ancestor, the determination of the coding capacity of the identified genes in the various species, and in some cases the determination of the presence of a shed HEMO protein after introduction of the cloned gene into an expression vector and transfection of 293T cells. The overall data are summarized in FIG. 10. We performed an in silico analysis of syntenic loci, by using the MultiPipMaker synteny building tool, between the RASL11B and USP46 genes, conserved in all mammalian genomes (and each found at about 120 kb from the human HEMO gene). Focus on the 15 kb HEMO region (FIG. 10A) shows that the HEMO gene entered the genome of mammals before the radiation of Laurasiatherians and Euarchontoglires, i.e. between 100-120 Mya (37), being found neither in Afrotherians (Elephant, Tenrec) nor in Xenarthrans (Armadillo). It also allowed the identification of the orthologous HEMO gene in primates (and as a very degenerate sequence in rodents) and, among Laurasiatherians in a series of ruminants and carnivores. Closer analysis further discloses that the HEMO gene has been conserved as a full-length protein-coding sequence in all simians (FIGS. 11A and 11B), and unexpectedly, in the cat (FIG. 12A-E). The identified full-length HEMO ORFs demonstrate high similarities, ranging from 84 to 99% amino acid identities (FIG. 10B, lower triangle) and show signs of purifying selection, with nonsynonymous to synonymous ratios (dN/dS) between all pairs of species lower than unity (mean value 0.46), except for very close species (e.g. human/chimpanzee) for which the number of mutations is not high enough to provide significant dN/dS values. For example, dN/dS values of 0.29-0.42 are observed between great apes and old world monkeys (OWM; FIG. 10B, upper triangle), as expected for a bona fide cellular gene.

To test the conservation of the specific shedding property observed in humans, a series of simian HEMO genes were cloned, introduced into the phCMV expression vector and tested by transfection of 293T cells as described above. As shown in FIG. 10C, the HEMO genes from all the tested species encode a protein which can be detected with the human HEMO antibodies (yet with a lower intensity for the distant New World Monkeys (NWM)), with in all cases evidence for protein shedding in the cell supernatant. Even in the NWM branch, where the HEMO protein has retained a functional furin site (see FIGS. 11A and 11B), a shed form of the protein is released in the supernatant, together with a smaller SU form. The smaller size observed for the Spider monkey protein is consistent with a small 10 aa deletion in the 5′ part of the gene (amino acid 182 to 191, FIGS. 11A and 11B). Accordingly, it appears that the shedding of the HEMO protein is a very well conserved property among simians, a feature which, together with the purifying selection applying to this gene, is a hint for a possible role of this secreted protein, notably in pregnant females. Of note, the domains 3′ to the shed protein form are much less conserved at the sequence level among simians, except for the transmembrane anchoring domain most probably required for shedding of the HEMO protein at the cell membrane (see FIGS. 11A and 11B).

### A Related HEMO Gene in Marsupials

To determine whether HEMO-like sequences could be present in some species where the orthologous gene could not be identified, a less stringent BLAST search was performed, which provided hits in Marsupials—but still neither in Afrotherians nor in Xenarthrans. Of note, the closest env gene identified is a conserved marsupial env gene that we had previously identified (Cornelis et al. 2015), namely env-panMars (see phylogenetic tree in FIG. 1D).

Amino acid sequence comparison of this conserved marsupial envelope protein with HEMO indicates only 20-30% similarity, but alignment of simian, cat and marsupial (from Opossum, Wallaby and Tasmanian Devil) sequences (FIG. 12A-E) shows significant identity regions, all along the extracellular domains. The env-panMars sequences correspond to truncated env due to a stop codon upstream of the transmembrane domain. The encoded proteins are therefore expected to be soluble proteins. As illustrated in FIG. 12D with HA-tagged env-panMars proteins, the Opossum and Wallaby env proteins are indeed released in the supernatant of cells transfected with the corresponding expression vectors. In the supernatant from Wallaby-transfected cells, a 15 kDa faint band can also be observed, which probably corresponds to the HA-tagged-TM subunit produced after partial cleavage at a degenerate furin site (FHKR). No similar band is observed for the Opossum (sequence at the furin site, VHKP).

Furthermore, RACE-PCR experiments performed on Wallaby RNA transcripts from ovary (FIG. 12E), locate the transcription start site within a CpG-rich region, with multiply-spliced RNAs in the promoter region, as observed for the HEMO gene. In the case of the Opossum, RNAseq data compiled in UCSC (FIG. 12E) show similar organization (with identical Transcription Start Site, located in a homologous CpG island and the use of the same E3 exon). Altogether, these data could indicate that both simian and marsupial env genes have a common retroviral ancestor, and that they probably correspond to the independent capture of related infectious retroviruses.

### Discussion

Here we have identified an endogenous retroviral envelope gene, HEMO, with a full-length protein-coding sequence, conserved in simians including humans, and with an unprecedented characteristic feature for a retroviral envelope since it is shed and released in the extracellular medium, being found at a high level in the blood of pregnant women. Several retroviral envelope gene “captures” have been reported, among most mammalian species, and in a number of cases these genes were demonstrated to be “syncytins”, i.e. genes playing a role in placentation, with the canonical immunosuppressive and fusogenic properties inherited from their ancestral retroviral progenitors being involved in a physiological function of benefit to the host (Mangeney et al. 2007; reviewed in Lavialle et al. 2013; Denner 2016). The presently identified HEMO gene shares some of the properties of syncytins, but is different, as it is shed in the extracellular environment with no evidence for fusogenic activity. In addition, its pattern of expression is not strictly restricted to the placenta—although it is the organ where its expression is highest. Yet, its conservation in evolution with characteristic features of a bona fide gene, i.e. evidence for purifying selection, together with the identification of a closely related retroviral env gene captured and conserved in the remote Marsupial clade (which diverged from eutherian mammals more than 150 Mya) sharing with HEMO a CpG-rich promoter and the capacity of its protein product to be released in the extracellular medium (in that case due to a stop codon located just upstream of the transmembrane domain of the TM subunit (Cornelis et al. 2015)), constitute a strong hint for a potential physiological role in simians (see below).

The identified retroviral env gene belongs to a poorly characterized and moderately reiterated ERV family, namely the MER34 family, with only highly degenerated elements (Vargiu et al. 2016; Toth and Jurka 1994; Jurka et al. 2005). Analysis of the structure of the genomic locus where HEMO can be identified only reveals traces of an ancestral provirus, with a highly rearranged gene organization. Of note, an LTR structure is only barely detectable 3′ to HEMO, and the 5′ LTR is no longer present. Actually, RACE-PCR analysis of the HEMO transcripts reveals a transcription start site within a CpG-rich domain, unrelated to a LTR, but clearly possessing a promoter activity as shown by transfection of reporter plasmids—with the promoter in both orientations—in cells in culture. This unusual promoter is most probably responsible for the specific pattern of expression of the HEMO gene, which is found to be active in a series of stem cells ex vivo, as well as in vivo very early in the developing embryo. The encoded protein itself has some unusual features, since it no longer possesses a furin cleavage site (although a functional one can still be demonstrated for the HEMO ortholog present within the New World Monkey genome), and more importantly because it is specifically cleaved at the cell membrane, via a metalloproteinase-mediated processing that results in the shedding of its ectodomain into the extracellular medium—observed for all simians including New World Monkeys. Shedding is a process that has not been reported previously for a retroviral envelope, although such a process is used by the cellular machinery for a series of cellular genes (e.g. Notch, TNF-alpha) involved for instance in signaling, cell mobility and migration. Of note, a closely related molecular event also takes place in the case of the Ebola filovirus envelope protein, which is in part shed in the cell medium by a specific ADAM-mediated cleavage upstream of the transmembrane domain. In that case also, the shed protein is detected in the blood, and is anticipated to play a critical role in the associated pathology, either by exerting a decoy effect on anti-Env antibodies, or even through direct immune activation and increased vascular permeability in the infected individuals. The presently observed shedding of the HEMO retroviral envelope protein de facto makes a link between unrelated viruses (e.g. a filovirus and a retrovirus). At the evolutionary level, this may be a hint for gene captures between distinct classes of viral elements, and/or of convergent evolution for the triggering of a systemic effect via a shedding process.

A further question concerns the possible role of HEMO in human physiology and/or pathology. Due i) to the high level of purifying selection acting on the gene in simians, ii) to the conservation in Marsupials of a gene transcribed from a similar promoter type and encoding a protein closely related in both sequence and mature protein structure, iii) to the rather uncommon profile of expression in development, and iv) to the massive shedding by the placenta of the protein into the blood, it can be anticipated that HEMO fulfils a role, most probably in pregnancy. A protective effect against infection by viruses and/or retroviruses would also be relevant. Such protective effects could be mediated by classical “interference”, via the sequestration of the receptor for the incoming virus, an effect which could be further enhanced by the release of the HEMO protein in the blood circulation and direct targeting of such receptors. Alternately, HEMO might possess a cytokine-like or hormone-like activity, with a possible role in pregnancy. An effect of HEMO in development should also be considered, taking into consideration that its expression is observed as early as at the 8-cell stage and persists at all the subsequent embryonic stages. Of note, other ERVs—including HERV-H and HERV-K—have related profiles of expression and abundant HERV-H RNA was recently demonstrated to be a marker of cell “stemness” in humans and to possibly play a role—via transcriptional effects and/or specific ERV-driven transcripts—in the maintenance of pluripotency in human stem cells. In the case of HEMO, which unambiguously encodes a retroviral envelope protein that can further be detected, its expression might not only be a “sternness” marker, as for the above highly reiterated ERVs, but its encoded protein might also constitute a molecular effector of pluripotency per se. Finally, we could unravel HEMO gene expression in a series of human tumors, and demonstrate HEMO protein expression in ovarian tumors. Further immunological analyses based on a large number of tumors and control tissues will have to be performed to definitely correlate HEMO protein expression with specific tumor histotypes for other retroviral Env expressed in ovarian tumors), and to assess whether this protein can be considered as a reliable marker of a given tumoral state and, tentatively, as a possible target for immuno-therapeutic approaches.

Experiments are now in progress to identify the cellular interacting partners of the HEMO protein, to further characterize HEMO functions in vivo, in both normal development and the onset of pathological processes.

### Example 2: mAb Production

Antibodies were produced by immunizing mice with a DNA fragment coding for 163 amino acids of the HEMO SU envelope subunit (aa 123 to 286; SEQ ID NO: 8), as described in example 1 above. Hybridoma 2F7 (IgG2a isotype), which is referred to in example 1 above, as well as other hybridoma were produced using standard cell fusion.

The hybridomas were deposited at the CNCM under the terms of the Budapest Treaty. CNCM is Collection Nationale de Culture de Microorganismes (Institut Pasteur; 28, rue du Docteur Roux; 75724 Paris CEDEX 15; France).

TABLE 6
mAb deposited at the CNCM

| | | | Antigen that has |
|---|---|---|---|
| | Number of CNCM | Date of CNCM | been used for Ab |
| Hybridoma | deposit | deposit | production |
| 2F7-E8 | I-5211 | Jun. 20, 2017 | SEQ ID NO: 8 |

A 121 amino acid HEMO ectodomain fragment—named HST5—from position 280 to 400 of SEQ ID NO: 1 is used as the antigen:

(SEQ ID NO: 988)
280-TWWLTGSNLTLSVNNSGLFFLCGNGVYKGFPPKWSGRCGLGYLVPS
LTRYLTLNASQITNLRSFIHKVTPHRCTQGDTDNPPLYCNPKDNSTIRAL
FPSLGTYDLEKAILNISKAMEQEFS-400

It is cloned in an eukaryotic expression vector as an N-terminal-Strep-tag (StrepTaglinker(GGGS)x3)-StrepTag)-HEMO fragment, and expressed in Drosophila cells S2. The antigen-StrepTag protein fragment is purified from the supernatant by a two-step method: first, on a Strep Tactin column and second, on a HiLoad 16/60 Superdex 75 column. Fractions are pooled and adjusted to 1 mg/ml, and used for immunization of mice and rats for 4 injections at Day0, D15, D45 and D60. Polyclonal antibodies production is tested by ELISA at Day25 and D55. Serum polyclonal antibodies are recovered after injections of the Streptag-HST5. Polyclonal antibodies are tested by Western Blot, ELISA and flow cytometry. Monoclonal Antibodies cloning are also done.

Sequence of the Streptag-HST5 peptide (SEQ ID NO:
989):
MTMITPSLHAGLCILLAVVAFVGLSLGASWSHPQFEKGGGSGGGSGGGSW
SHPQFEKGADDDDKTGTWWLTGSNLTLSVNNSGLFFLCGNGVYKGFPPKW
SGRCGLGYLVPSLTRYLTLNASQITNLRSFIHKVTPHRCTQGDTDNPPLY
CNPKDNSTIRALFPSLGTYDLEKAILNISKAMEQEFS
Strep Tag: in italic
Linker: in bold
Hemo-HST5 sequence aa 280 to 400: underlined

### Example 3: Production of Antibodies that (Specifically) Bind to the Membrane-Attached Portion of the HEMO Ectodomain [That is Retained at the Cell Surface after Shedding of the Soluble Fragment]

A DNA fragment coding for 57 amino acids of the ectodomain part of the HEMO protein (aa 433 to 489; SEQ ID NO: 990), corresponding to a post-SHED fragment, can be inserted into the pET28b and expressed in BL21 bacteria as described in Example 1, and the recombinant protein fragment used to immunize mice.

Synthetic peptides (10 to 20 amino acids) corresponding to portions of the membrane-attached ectodomain protein (aa 433 to 489; SEQ ID NO: 990) can be synthetized and conjugated to carrier protein, such as KLH (keyhole limpet hemocyanin). Peptides are administered to mice for immunization.

The antibodies produced are collected. Hybridomas are produced using standard cell fusion.

### Example 4: Screening/Histotyping of a Large Panel of Tumors

The HEMO protein can be considered as a potential cancer biomarker and promising therapeutic target. Samples of tumor tissues are screened for the presence of the HEMO protein, by immunohistochemistry, according to the protocol described in Example 1, more particularly for the presence of a (N-terminal) soluble fragment of HEMO ectodomain and/or for the presence of a membrane-anchored (C-terminal) fragment of HEMO domain HEMO (fragment, which is retained at the cell surface after shedding of the soluble fragment). The antibodies, more particularly monoclonal antibodies, of examples 2 and/or 3 can be used for this detection. Control (non-tumoral) tissues are screened in parallel.

The tumor tissues comprise in a non-limitative way:
Ovarian
Uterine: Endometrial, Cervical, Gestational (Choriocarcinoma)
Breast
Lung
Colon
Germ cell
Head and Neck
Bone marrow

The cells expressing HEMO can be isolated from fresh tumoral samples (more particularly biopsies), using FACS analysis with Antibodies described in example 2 and 3, and further analyzed for cell marker identification, more particularly for identification of stem cell marker(s). Control (non-tumoral) cells are analyzed in parallel.

### Example 5: Optimization of a Blood Detection Test (for Diagnostic, Pronostic and Evolution)

An Elisa assay can be used to detect variability in the HEMO sera level, in normal and pathological conditions and/or follow the evolution in pathological conditions. Such an assay may comprise antibodies described in Example 2.

### Example 6: HEMO Protein Expression in Tumors

### 1—Microarray

Method

In silico analysis of microarray was performed, as indicated in Exemple 1 (Microarray data mining), on additional data from the Expression project for Oncology (expO) dataset (GEO accessions GSE2109), and boxplot representations were plotted according to the tumor primary site.

Results

Microarray dataset analyses (FIGS. 9A and 9B, and FIG. 14) show heterogenous expression in many tumor types. This is also observed with the TCGA RNAseq dataset of NIH-GDC Data Portal (FIG. 15A, https://portal.gdc.cancer.gov/projects).

In summary, tumors with higher HEMO expression are
gynecological cancer:
   ovary: histological sub-type “Clear Cell Carcinoma” and “Endometrioid”
   uterus: endometrial and cervical cancer
breast cancer
lung cancer
digestive cancer: colorectal, stomach, liver
head and neck cancer
germ cell cancer
urothelial cancer (including bladder)
bone marrow cancer,
   and to a less extend, tumors from kidney, prostate and brain.

Heterogeneity may depend on the amount of cancer cells in the samples and on the tumor stage.

### 2—TGCA RNAseq

Method

FastQ files from TCGA RNAseq-tumors were downloaded from the TCGA site FastQ files from TCGA RNAseq-tumors were downloaded from the TCGA site portal.gdc.cancer.gov/projects and reads for HEMO and a set of House-Keeping genes were quantified, and normalized using the R-DESeq2 package. Hemo expression is shown as boxplots using the R ggplot2 package.

Results

FIG. 15B, I: Number of cases of Control and Tumoral tissues are indicated. Each dot represents a case.

FIG. 15B, II: Boxplot enlargments are shown, and exclude the highest values.

Box plots show high and heterogeneous HEMO expression in tumors, compared to the controlateral normal tissues in HNSC and UCEC. Heterogeneity is observed for HEMO expression in the 3 dataset with highest values in UCEC dataset (FIG. 15B).

3—HEMO Expression in Tumors Samples from Gustave Roussy

Methods

Analysis was performed on a representative panel (20 to 30 samples for each tumor type: Ovary, Uterus, Breast and Head & Neck) as follows:
Frozen samples of tumor tissues and normal control tissue were processed as indicated:
   Western Blot (see Example 1): protein extraction was done on 20 cryosections of 50 μM and monoclonal antibody 2F7 (CNCM I-5211) was used;
   Tissue staining (Hematoxylin Eosin Saffron) was performed on control cryosections of 5 μm; and
Immunochemistry (IHC) was done with monoclonal antibody 2F7 (CNCM I-5211) on corresponding Formalin-Fixed Paraffin-Embedded (FFPE) samples as follows: paraffin sections were processed for heat-induced antigen retrieval (Tris-EDTA, pH 9; Abcam) and incubated overnight with the monoclonal mouse anti-HEMO (2F7, CNCM I-5211) antibody (1/10 dilution) or a control IgG2a isotype. Staining was visualized by using the peroxidase/diaminobenzidine Mouse PowerVision® kit (ImmunoVision Technologies®).

Results

### Ovarian Carcinomas (FIG. 16A, 16B)

Western blot analysis of protein extracts (using 2F7 mAB) shows expression of HEMO in Placenta sample and in Ovarian Endometrioid (E) and Clear Cell Carcinomas (C1 to C5) samples. No expression is detected in normal ovarian tissues (N1 and N2). Membrane was rehybridized with an anti-tubulin Ab to quantify protein load in the samples.

Immunohistochemical analysis (using the 2F7 mAb) of formalin-fixed Ovarian Endometrioid tumors from two patients, shows high HEMO expression in specific tumor cells. Details in HES sections of patient II show heterogeneity in tumor cells.

### Uterine Carcinomas (FIG. 17)

HES and IHC (using the 2F7 mAb) at different magnifications show specific expression of the HEMO protein in specific tumor cells of Endometrial Carcinomas from two patients. No HEMO expression is detected in the normal tissues on the same sections.

### Breast Carcinomas (FIG. 18)

HES and IHC (using the 2F7 mAb) at different magnifications show no HEMO expression in normal controlateral breast tissue and show HEMO expression at various levels in two patients with Breast tumors of different molecular signatures: high staining is observed in tumor cells of a HER2+ Breast Carcinoma and more diffuse staining is observed in a Triple Neg Breast Carcinoma.

### 4—Characterization of HEMO Positive Tumoral Cells

HEMO being express in stem cells (ES and iPSC) and in placental cytotrophoblastic cells, both being highly proliferative, HEMO positive cells is isolated from tumors to characterize their potential proliferative properties. Tumor-HES and IHC (see example 6, part 3) showed specific morphology of the HEMO positive cells in the tumoral samples, these cells can be targeted by specific antibodies (drug conjugated mono or bispecific).

To investigate the potential and utility of targeting HEMO positive cells in a tumor, these cells are isolated and sorted by flow cytometer, with the anti-HEMO antibodies described in Examples 2 and 3, then their proliferative status is analyzed, compared to the HEMO negative cells. RNAseq analyses were performed on HEMO positive and negative cells, in different tumor types, and compared to search for specific molecular pathways, and expression of stem cell markers. Proliferative properties are also investigated in ex vivo models and in PDX-mice.

### Example 7: Development of a Blood-ELISA Assay for Detection of Circulating HEMO Shed Protein

Under physiological conditions, the protein is detected by Western blot on deglycosylated samples of human sera, and the level rise during pregnancy (FIG. 6). The aim was to develop a sensitive assay to detect variation in the serum level of patient with HEMO-positive tumors, or in women with pathological pregnancy.

A sandwich ELISA test was developed (FIG. 19A) which consists of at least one purified monoclonal antibody coated at a high concentration (200 ng of capture antibodies in each well of Maxisorp® plate), in order to capture the serum HEMO shed protein, and a second polyclonal or monoclonal antibody, against a different epitope of the protein to detect the captured HEMO.

Method

### Reagents:

PBST: PBS 1×+0.1% Tween 20
BSA (Bovine Serum Albumin)
Capture antibodies (a rabbit Hemo-TM-capture-polyclonal antibody, SIGMA-ALDRICH®, Product Name=Anti-ERVMER34-1)
Primary antibodies (see Example 1: mouse anti-HEMO polyclonal antibody), or purified 2F7 mAb, or supernatant of hybridoma, or ScFv HIS-tag or rabbit-Fc mAbs
Secondary antibodies (anti-mouse HRP), or anti-HIS HRP or anti-rabbit HRP
Revelation solution TMB
Phosphoric acid 1M

### Materials:

Maxisorp® plate (Nunc, ThermoFischer®)
Pipettes
Biotek® plate reader
Sealing films

### Steps:

### D−1:

Coat 200 ng of capture antibodies in each well of Maxisorp plate
Seal the plate and incubate overnight at 4° C.

### D0:

Keep the coating solution
Saturate the plate with 100 μL of PBST (PBS 1×+0.1% Tween)+5% BSA and incubate during 1 h at room temperature
Add 50 μL of antigen solution (generally supernatant of HH1 transfected cells diluted in SVF, ratio 1:50) or samples or sera samples in each well
Wash the plate 3 times with 300 μL of PBST in each well
Prepare a primary antibody solution in PBST+1% BSA (1/1000)
Add 50 μL of primary antibody solution in each well and incubate at room temperature during at least 1 h
Wash the plate 3 times with 300 μL of PBST in each well
Prepare a secondary antibody solution in PBST+1% BSA (1/5000)
Add 50 μL of secondary antibody solution in each well and incubate at room temperature during 45 min
Wash the plate 3 times with 300 μL of PBST in each well
Add 50 μL of revelation solution after mixing solution A to solution B (ratio 1:1)
Add 50 μL of phosphoric acid (1 M) to stop the reaction
Read the plate at 450 nm with a plate reader

Results

The curve obtained with ELISA assay (FIG. 19B) showed the same result as the detection of HEMO by Western Blot on peripheral blood of pregnant women (FIG. 6): more time of pregnancy is high, more HEMO is detected.

### Example 8: Antibodies Raised Against the C-Terminal Part of the HEMO-Ectodomain

After shedding, the C-terminal part of the Ectodomain, namely between the major shedding sites (432-433 of SEQ ID NO: 1) and the beginning of the transmembrane domain (around position 492 of SEQ ID NO: 1) is still present at the extracellular side of the cell membrane, anchored by the downstream transmembrane region of HEMO. This N-terminal part of the post-SHED-HEMO is accessible to specific antibodies.

To target HEMO tumoral producing cells with high efficiency, an antibody was developed for further drug conjugate Ab-targeting.

Method

A 85 amino acid HEMO ectodomain fragment—named HTM5—from position 387 to 471 of SEQ ID NO: 1, namely on both sides of the shedding site QR, is used as the antigen:

(SEQ ID NO: 919)
387-AILNISKAMEQEFSATKQTLEAHQSKVSSLASASRKDHVLDIPTTQ
RQTACGTVGKQCCLYINYSEEIKSNIQRLHEASENLKNV-471

It was cloned in an eukaryotic expression vector as an N-terminal-Strep-tag (StrepTaglinker(GGGS)x3)-StrepTag)-HEMO fragment, and expressed in Drosophila cells S2. The HTM5-StrepTag protein fragment was purified from the supernatant by a two-step method: first, on a Strep Tactin column and second, on a HiLoad 16/60 Superdex 75 column. Fractions were pooled and adjusted to 1 mg/ml, and used for immunization of mice and rats for 4 injections at Day0, D15, D45 and D60. Polyclonal antibodies production was tested by ELISA at Day25 and D55. Serum polyclonal antibodies were recovered after injections of the Streptag-HTM5. Polyclonal antibodies were tested by Western Blot, ELISA and flow cytometry. The serum of one mice containing polyclonal antibodies against the StrepTag-HTM5 peptide alone was used in the experiments (FIGS. 20A and 20B). Monoclonal Antibodies cloning are also done.

Sequence of the Streptag-HTM5 peptide (SEQ ID NO:
920):
MTMITPSLHAGLCILLAVVAFVGLSLGASWSHPQFEKGGGSGGGSGGGSW
SHPQFEKGADDDDKTGAILNISKAMEQEFSATKQTLEAHQSKVSSLASAS
RKDHVLDIPTTQRQTACGTVGKQCCLYINYSEEIKSNIQRLHEASENLKN
V
Strep Tag: in italic
Linker: in bold
Hemo-HTM5 sequence aa 387 to 471: underlined

To test if the mouse pAb-antiHTM5 can detect the native form of the protein, and both side of the shedding site, namely the C-term of the Shed-HEMO and the N-term of the membrane attached-HEMO, various HEMO producing vectors were constructed and transfected in 293T cells:

| | |
|---|---|
| 1. | the full-length HEMO-pHCMV vector |
| 2. | a SU-HEMO-pHCMV vector (aa 1 to 351 of SEQ ID |
| | NO: 1 = SEQ ID NO: 921) |
| 3. | a TM-HEMO-pHCMV vector (internal deletion from |
| | aa 34 to 352 = 1-33 + 353-563 from SEQ ID NO: |
| | 1): |
| | MGSLSNYALLQLTLTAFLTILVQPQHLLAPVFRTQGDTDNPPLYCN |
| | PKDNSTIRALFPSLGTYDLEKAILNISKAMEQEFSATKQTLEAHQS |
| | KVSSLASASRKDHVLDIPTTQRQTACGTVGKQCCLYINYSEEIKSN |
| | IQRLHEASENLKNVPLLDWQGIFAKVGDWFRSWGYVLLIVLFCLFI |
| | FVLIYVRVFRKSRRSLNSQPLNLALSPQQSAQLLVSETSCQVSNRA |
| | MKGLTTHQYDTSLL (SEQ ID NO: 922) |
| 4. | a post-SHED-HEMO-pHCMV vector (internal |
| | deletion from aa 34 to 432 = 1-33 + 433-563 |
| | from SEQ ID NO: 1): |
| | MGSLSNYALLQLTLTAFLTILVQPQHLLAPVFRRQTACGTVGKQCC |
| | LYINYSEEIKSNIQRLHEASENKNVPLLDWQGIFAKVGDWFRSWGY |
| | VLLIVLFCLFIFVLIYVRVFRKSRRSLNSQPLNLALSPQQSAQLLV |
| | SETSCQVSNRAMKGLTTHQYDTSLL (SEQ ID NO: 923) |

After transfections, either cell lysates were analyzed by Western blot with the HTM5-pAB (FIG. 20A) or cells were analyzed by flow cytometry with the mouse pAB-HTM5 (FIG. 20B).

Immunizations of mice and rats are also done with other human HEMO ectodomain fragments (see below) linked to KLH (keyhole limpet hemocyanin) protein carrier thanks to a cystein amino acid at the C-terminal extremity of said human HEMO ectodomain fragments:

TABLE 7
Human HEMO ectodomain fragments used to
produce antibodies raised against the
C-terminal part of the HEMO-ectodomain.

| From | To | | SEQ |
|---|---|---|---|
| amino | amino | human HEMO ectodomain | ID |
| acid | acid | fragments | NO: |
| 466 | 492 | ENLKNVPLLDWQGIFAKVGDWFRSWGY | 924 |
| 466 | 491 | ENLKNVPLLDWQGIFAKVGDWFRSWG | 925 |
| 466 | 490 | ENLKNVPLLDWQGIFAKVGDWFRSW | 926 |
| 466 | 489 | ENLKNVPLLDWQGIFAKVGDWFRS | 927 |
| 466 | 488 | ENLKNVPLLDWQGIFAKVGDWFR | 928 |
| 467 | 492 | NLKNVPLLDWQGIFAKVGDWFRSWGY | 929 |
| 467 | 491 | NLKNVPLLDWQGIFAKVGDWFRSWG | 930 |
| 467 | 490 | NLKNVPLLDWQGIFAKVGDWFRSW | 931 |
| 467 | 489 | NLKNVPLLDWQGIFAKVGDWFRS | 932 |
| 467 | 488 | NLKNVPLLDWQGIFAKVGDWFR | 933 |
| 468 | 492 | LKNVPLLDWQGIFAKVGDWFRSWGY | 934 |
| 468 | 491 | LKNVPLLDWQGIFAKVGDWFRSWG | 935 |
| 468 | 490 | LKNVPLLDWQGIFAKVGDWFRSW | 936 |
| 468 | 489 | LKNVPLLDWQGIFAKVGDWFRS | 937 |
| 468 | 488 | LKNVPLLDWQGIFAKVGDWFR | 938 |
| 468 | 486 | LKNVPLLDWQGIFAKVGDW | 939 |
| 465 | 492 | SENLKNVPLLDWQGIFAKVGDWFRSWGY | 981 |
| 465 | 491 | SENLKNVPLLDWQGIFAKVGDWFRSWG | 982 |
| 465 | 490 | SENLKNVPLLDWQGIFAKVGDWFRSW | 983 |
| 465 | 489 | SENLKNVPLLDWQGIFAKVGDWFRS | 984 |
| 465 | 488 | SENLKNVPLLDWQGIFAKVGDWFR | 985 |
| 465 | 484 | SENLKNVPLLDWQGIFAKVG | 986 |
| 473 | 492 | LLDWQGIFAKVGDWFRSWGY | 987 |

Results

The HTM5 antibody (mouse polyclonal anti aa 387-471 of SEQ ID NO: 1=SEQ ID NO: 919) can detect the native form of (FIG. 20A-20B):
the full length-HEMO
the TM part of HEMO and not the SU
the C-terminal part of the ectodomain (aa 433-471).

### Example 9: KO (Knock-Out) Cell Clones for HEMO by CrispR-Cas9

CaCo-2 CELL LINE

Development of the CrispR technique on the CaCo-2 cell line, human colon adenocarcinoma, strongly expressing HEMO (see FIG. 2B):
1. transfection of plasmids containing the Cas9 gene, and various RNA-guides for targeting the gene HEMO (4 different regions chosen);
2. Obtaining KO clones on the two alleles, which no longer expressing the HEMO protein, by limiting dilution; and
3. Verification by sequencing clones DNA: mutation of the ORF (nucleotide indel with frame-shift and premature stop codon); and
4. Verification by IHC with monoclonal antibody 2F7 (CNCM I-5211) on FFPE block cells and by Western Blot on concentrated supernatant of WT (Wild Type) and KO cells in culture.

Induced Pluripotent Stem Cell (iPSC)

Obtaining several iPSC KO clones on the two alleles in several steps:
1. electroporation of the Cas9-Guide plasmid, with the 2 best guides (among the 4 tested with CaCo-2);
2. Verification of mutations by sequencing the HEMO gene;
3. Verification of the absence of protein in the concentrated culture supernatant of iPSC-KO clones; and
4. Verification of the pluripotency of KO clones obtained by the capacity
   to develop embryoid bodies in culture,
   and teratomas in NSG (Nod Scid Gamma) mice.

Result

Both CaCo-2 (FIG. 21A, 21B) and iPSC (FIG. 21C) allow the development of KO HEMO cells as it confirmed by IHC and Western Blot analyses. It has to be pointed out that these KO clones (CaCo-2 or iPSC) represent tools to study:
the role of HEMO (transcriptomic analysis of RNAseq of WT cells and KO cells: search for genes whose expression is modulated by HEMO); and
the effect of specific drugs directed against the protein, in cultured cell systems or mouse xenografts (PDX).

### Example 10: Method for Detecting a Defect in Placentation

Variations of expression of the HEMO protein in the blood of pregnant women are evaluated, to see if there is a correlation with pathologies pregnancy (Intra-Uterine Growth Delay, Pre-Eclampsia, etc.).

The cohort includes 200 samples of pregnant women.

It obtains the blood samples from pregnant women, around the 28^{th} WA (Week of Amenorrhea), during the second trimester sampling (6^{th} month):
one dry tube on gel, to recover the serum (analysis of the serum level of the HEMO protein produced by the placenta by Western Blot and/or ELISA test);
optional: one EDTA tube, to recover maternal DNA from cells lymphocytes (verification of the HEMO gene sequence).

It is also possible
to return to the medical file, looking for associated pathologies,
to obtain a second blood sample (current 3^{rd} trimester, or pre-delivery), to check the serum level of HEMO,
in the event of an abnormality, to obtain at delivery a fragment of placental tissue or of fetal cord to analyze the fetal DNA, to check the HEMO gene sequence (the protein being produced by the placenta, of fetal origin).

The pregnant woman have signed a consent for the research on the blood samples and have given the following desired information:
whether it is a twin pregnancy or not, and whether it is a pregnancy mono- or bi-chorial,
if there is already a known pathology in the progress of the pregnancy and current infections (HIV in particular),
the number of children, and
other pathologies (tumor, other)

### Example 11: Cloning of the mAB as ScFv Fragments

Method

One mAbs against the SU domain have been obtained (2F7, CNCM I-5211, see Example 2), which is a mouse IgG2a antibodies. For future use (ELISA blood tests, cristallography experiments, and putative cancer targeting) this mAbs were engineered and cloned as scFv fragments as follows:
1. Cloning of the ScFv as VH-(linker{GGGGS}x4)-Vkappa fragments from RNAs of hybridoma Ab;
2. producing cell lines, by conventional RACE method;
3. Sequence determination; and
4. Production and verification of ScFv binding to the HEMO protein, by Western Blot, ELISA, flow cytometry, or IHC tests.

Result

ELISA binding (FIG. 22A) and flow cytometry binding (FIG. 22B) of ScFv-2F7-Fc and ScFv-2F7-His to the SHED-HEMO protein produced in supernatant of 293T transfected cells, compared to empty vector transfected supernatants, showed that the scFV fragment works as the full 2F7 antibody (CNCM I-5211).

### Sequences

The sequences are described by reference to the Figures and to the Examples. A ST25-sequence listing is also attached.

TABLE 8
sequences

| SEQ ID NO: | | |
|---|---|---|
| 1 | Human HEMO protein | Human sequence (FIG. 1C) |
| 129-142 | Non-human HEMO proteins | Boreoeutheria mammals (Euarchontoglies) |
| | Non-human Boreoeutheria mammals | CPZ, GOR, ORA, GIB, MAC, BAB, AGM, COL, LAN, RHI, MAR, SQM, SPI, SAK |
| | | cf. FIGS. 11A, 11B, 12A, 12B |
| 143 | | Boreoeutheria mammals (Laurasiathera) |
| | | CAT |
| | | cf. FIGS. 12A, 12B |
| 144-146 | Marsupial env-panMars proteins | Marsupial mammals |
| | | OPO, WAL, TAS |
| | | cf. FIGS. 12A, 12B |
| 2 | Human HEMO signal peptide | 1-26 from SEQ ID NO: 1 |
| 168 | End position 26, 24 or 25 | 1-24 from SEQ ID NO: 1 |
| 169 | | 1-25 from SEQ ID NO: 1 |
| 147 | Generic signal peptide for HEMO protein of | MX⁷SLX⁸X⁹YX¹⁰LLX¹¹LX¹²X¹³TAX¹⁴LTX¹⁵X¹⁶VQX¹⁷QH |
| | Boreoeutheria mammals | wherein |
| | HUM, CPZ, GOR, ORA, GIB, MAC, BAB, AGM, | X⁷ = G, X⁸ = S, X⁹ = N, X¹⁰ = A, X¹¹ = Q, X¹² = T, X¹³ = L, X¹⁴ = F, X¹⁵ = I, X¹⁶ = L, and X¹⁷ = P [=SEQ |
| | COL, LAN, RHI, MAR, SQM, SPI, SAK | ID NO: 2]; or |
| | Cf. FIG. 11A | X⁷ = G, X⁸ = S, X⁹ = N, X¹⁰ = A, X¹¹ = Q, X¹² = T, X¹³ = F, X¹⁴ = F, X¹⁵ = I, X¹⁶ = L, and X¹⁷ = P; or |
| | end position = aa position 26, 24 or 25 | X⁷ = V, X⁸ = S, X⁹ = N, X¹⁰ = A, X¹¹ = Q, X¹² = T, X¹³ = L, X¹⁴ = F, X¹⁵ = I, X¹⁶ = L, and X¹⁷ = A; or |
| | | X⁷ = V, X⁸ = S, X⁹ = D, X¹⁰ = A, X¹¹ = Q, X¹² = T, X¹³ = L, X¹⁴ = F, X¹⁵ = I, X¹⁶ = L, and X¹⁷ = A; or |
| | | X⁷ = V, X⁸ = S, X⁹ = N, X¹⁰ = A, X¹¹ = Q, X¹² = T, X¹³ = L, X¹⁴ = F, X¹⁵ = T, X¹⁶ = L, and X¹⁷ = A; or |
| | | X⁷ = G, X⁸ = L, X⁹ = N, X¹⁰ = G, X¹¹ = P, X¹² = M, X¹³ = L, X¹⁴ = L, X¹⁵ = I, X¹⁶ = Q, and X¹⁷ = P; or |
| | | X⁷ = G, X⁸ = S, X⁹ = N, X¹⁰ = G, X¹¹ = Q, X¹² = T, X¹³ = L, X¹⁴ = L, X¹⁵ = I, X¹⁶ = R, and X¹⁷ = P; or |
| | | X⁷ = D, X⁸ = S, X⁹ = N, X¹⁰ = V, X¹¹ = Q, X¹² = T, X¹³ = L, X¹⁴ = L, X¹⁵ = I, X¹⁶ = R, and X¹⁷ = P; or |
| | | X⁷ = G, X⁸ = S, X⁹ = N, X¹⁰ = V, X¹¹ = Q, X¹² = T, X¹³ = L, X¹⁴ = L, X¹⁵ = I, X¹⁶ = R, and X¹⁷ = P |
| 405 | | MX⁷SLX⁸X⁹YX¹⁰LLX¹¹LX¹²X¹³TAX¹⁴LTX¹⁵X¹⁶VQX¹⁷ |
| 406 | | MX⁷SLX⁸X⁹YX¹⁰LLX¹¹LX¹²X¹³TAX¹⁴LTX¹⁵X¹⁶VQX¹⁷Q |
| 3 | Human HEMO protein without the signal peptide | 27-563 from SEQ ID NO: 1 |
| 170 | Start position 27, 25 or 26 | 25-563 from SEQ ID NO: 1 |
| 171 | End position 563 | 26-563 from SEQ ID NO: 1 |
| 4 | Human HEMO ectodomain | 27-489 from SEQ ID NO: 1 |
| 172 | (without the signal peptide) | 25-489 from SEQ ID NO: 1 |
| 173 | start position 27, 25 or 26 | 26-489 from SEQ ID NO: 1 |
| 174 | end position 489, 488, 491 or 486 | 27-488 from SEQ ID NO: 1 |
| 175 | | 25-488 from SEQ ID NO: 1 |
| 176 | | 26-488 from SEQ ID NO: 1 |
| 177 | | 27-491 from SEQ ID NO: 1 |
| 178 | | 25-491 from SEQ ID NO: 1 |
| 179 | | 26-491 from SEQ ID NO: 1 |
| 180 | | 27-486 from SEQ ID NO: 1 |
| 181 | | 25-486 from SEQ ID NO: 1 |
| 182 | | 26-486 from SEQ ID NO: 1 |
| 438 | Human HEMO signal peptide and ectodomain | 1-489 from SEQ ID NO: 1 |
| 439 | end position 489, 488, 491 or 486 | 1-488 from SEQ ID NO: 1 |
| 440 | | 1-491 from SEQ ID NO: 1 |
| 441 | | 1-486 from SEQ ID NO: 1 |
| 442-456 | Non-human HEMO ectodomain | Ectodomain corresponding to positions 27-489 of human ectodomain (SEQ ID NO: 4) |
| 457-471 | (without the signal peptide) | Ectodomain corresponding to positions 25-489 of human ectodomain |
| 472-486 | non-human Boreoeutheria mammals | Ectodomain corresponding to positions 26-489 of human ectodomain |
| 487-501 | CPZ, GOR, ORA, GIB, MAC, BAB, AGM, COL, | Ectodomain corresponding to positions 27-488 of human ectodomain |
| 502-516 | LAN, RHI, MAR, SQM, SPI, SAK, CAT | Ectodomain corresponding to positions 25-488 of human ectodomain |
| 517-531 | cf. FIGS. 11A, 11B, 12A, 12B and 12C | Ectodomain corresponding to positions 26-488 of human ectodomain |
| 532-546 | | Ectodomain corresponding to positions 27-491 of human ectodomain |
| 547-561 | | Ectodomain corresponding to positions 25-491 of human ectodomain |
| 562-576 | | Ectodomain corresponding to positions 26-491 of human ectodomain |
| 577-591 | | Ectodomain corresponding to positions 27-486 of human ectodomain |
| 592-606 | | Ectodomain corresponding to positions 25-486 of human ectodomain |
| 607-621 | | Ectodomain corresponding to positions 26-486 of human ectodomain |
| 7 | Human HEMO ImmunoSuppressive Domain | 420-436 from SEQ ID NO: 1 |
| | (ISD) | |
| 148 | Generic ISD for HEMO protein of | SX¹⁸X¹⁹DX²⁰VLDX²¹PTTQRQTA |
| | Boreoeutheria mammals | wherein |
| | HUM, CPZ, GOR, ORA, GIB, MAC, BAB, AGM, | X¹⁸ = R, X¹⁹ = K, X²⁰ = H, and X²¹ = I [=SEQ ID NO: 7]; or |
| | COL, LAN, RHI, MAR, SQM, SPI, SAK | X¹⁸ = Q, X¹⁹ = K, X²⁰ = H, and X²¹ = I; or |
| | Cf. FIG. 11B | X¹⁸ = P, X¹⁹ = N, X²⁰ = R, and X²¹ = I; or |
| | | X¹⁸ = P, X¹⁹ = N, X²⁰ = R, and X²¹ = L |
| 410 | Human HEMO C-X6-CC motif | CGTVGKQCC |
| 149 | Generic C-X6-CC motif for HEMO protein of | CX²²X²³X²⁴X²⁵X²⁶X²⁷CC |
| | Boreoeutheria mammals | wherein |
| | HUM, CPZ, GOR, ORA, GIB, MAC, BAB, AGM, | X²² = G, X²³ = T, X²⁴ = V, X²⁵ = G, X²⁶ = K and X²⁷ = Q; or |
| | COL, LAN, RHI, MAR, SQM, SPI, SAK, CAT | X²² = R, X²³ = T, X²⁴ = V, X²⁵ = G, X²⁶ = K and X²⁷ = Q; or |
| | Cf. FIG. 11B | X²² = G, X²³ = T, X²⁴ = V, X²⁵ = D, X²⁶ = K and X²⁷ = Q; or |
| | | X²² = T, X²³ = I, X²⁴ = V, X²⁵ = G, X²⁶ = N and X²⁷ = Q |
| 187 | Generic C-X7-CC motif for retroviral Env | |
| 188 | Generic C-X5-CC motif for retroviral Env | |
| 5 | Human HEMO transmembrane domain | 490-512 from SEQ ID NO: 1 |
| 427 | start position 490, 489, 492 or 487 | 489-512 from SEQ ID NO: 1 |
| 428 | end position 512, 509 or 513 | 492-512 from SEQ ID NO: 1 |
| 429 | | 487-512 from SEQ ID NO: 1 |
| 430 | | 490-509 from SEQ ID NO: 1 |
| 431 | | 489-509 from SEQ ID NO: 1 |
| 432 | | 492-509 from SEQ ID NO: 1 |
| 433 | | 487-509 from SEQ ID NO: 1 |
| 434 | | 490-513 from SEQ ID NO: 1 |
| 435 | | 489-513 from SEQ ID NO: 1 |
| 436 | | 492-513 from SEQ ID NO: 1 |
| 437 | | 487-513 from SEQ ID NO: 1 |
| 151 | Generic transmembrane domain for HEMO | WGYVX²⁹LIVX³⁰FCLX³¹IFVLX³²YX³³X³⁴X³⁵F, |
| | protein of Boreoeutheria mammals | wherein |
| | HUM, CPZ, GOR, ORA, GIB, MAC, BAB, AGM, | X²⁹ is L; X³⁰ is L; X³¹ is F; X³² is I; X³³ is V; X³⁴ is R; and X³⁵ is V [=SEQ ID NO: 5]; or |
| | COL, LAN, RHI, MAR, SQM, SPI, SAK | X²⁹ is L; X³⁰ is L; X³¹ is F; X³² is I; X³³ is V; X³⁴ is H; and X³⁵ is V; or |
| | Cf. FIG. 11B | X²⁹ is L; X³⁰ is L; X³¹ is F; X³² is I; X³³ is V; X³⁴ is H; and X³⁵ is I; or |
| | start position = 490, 489, 492 or 487 | X²⁹ is L; X³⁰ is F; X³¹ is I; X³² is I; X³³ is V; X³⁴ is R; and X³⁵ is F; or |
| | end position = 512 | X²⁹ is L; X³⁰ is F; X³¹ is I; X³² is I; X³³ is I; X³⁴ is R; and X³⁵ is F; or |
| | | X²⁹ is L; X³⁰ is F; X³¹ is I; X³² is T; X³³ is V; X³⁴ is R; and X³⁵ is F; or |
| | | X²⁹ is F; X³⁰ is F; X³¹ is I; X³² is I; X³³ is V; X³⁴ is R; and X³⁵ is F |
| 407 | | SWGYVX²⁹LIVX³⁰FCLX³¹IFVLX³²YX³³X³⁴X³⁵F |
| 408 | | YVX²⁹LIVX³⁰FCLX³¹IFVLX³²YX³³X³⁴X³⁵F |
| 409 | | FRSWGYVX²⁹LIVX³⁰FCLX³¹IFVLX³²YX³³X³⁴X³⁵F |
| 419 | Generic transmembrane domain for HEMO | WGYVX²⁹LIVX³⁰FCLX³¹IFVLX³²YX³³, |
| | protein of Boreoeutheria mammals | wherein |
| | Cf. FIG. 11 | X²⁹ is L; X³⁰ is L; X³¹ is F; X³² is I; X³³ is V; or |
| | HUM, CPZ, GOR, ORA, GIB, MAC, BAB, AGM, | X²⁹ is L; X³⁰ is L; X³¹ is F; X³² is I; X³³ is V; or |
| | COL, LAN, RHI, MAR, SQM, SPI, SAK | X²⁹ is L; X³⁰ is L; X³¹ is F; X³² is I; X³³ is V; or |
| | start position = 490, 489, 492 or 487 | X²⁹ is L; X³⁰ is F; X³¹ is I; X³² is I; X³³ is V; or |
| | end position = 509 | X²⁹ is L; X³⁰ is F; X³¹ is I; X³² is I; X³³ is I; or |
| | | X²⁹ is L; X³⁰ is F; X³¹ is I; X³² is T; X³³ is V; or |
| | | X²⁹ is F; X³⁰ is F; X³¹ is I; X³² is I; X³³ is V |
| 420 | | SWGYVX²⁹LIVX³⁰FCLX³¹IFVLX³²YX³³ |
| 421 | | YVX²⁹LIVX³⁰FCLX³¹IFVLX³²YX³³ |
| 422 | | FRSWGYVX²⁹LIVX³⁰FCLX³¹IFVLX³²YX³³ |
| 423 | Generic transmembrane domain for HEMO | WGYVX²⁹LIVX³⁰FCLX³¹IFVLX³²YX³³X³⁴X³⁵FX⁵⁰, |
| | protein of Boreoeutheria mammals | wherein |
| | HUM, CPZ, GOR, ORA, GIB, MAC, BAB, AGM, | X²⁹ is L; X³⁰ is L; X³¹ is F; X³² is I; X³³ is V; X³⁴ is R; X³⁵ is V; and X⁵⁰ is R or G; or |
| | COL, LAN, RHI, MAR, SQM, SPI, SAK | X²⁹ is L; X³⁰ is L; X³¹ is F; X³² is I; X³³ is V; X³⁴ is H; X³⁵ is V; and X⁵⁰ is R; or |
| | Cf. FIG. 11B | X²⁹ is L; X³⁰ is L; X³¹ is F; X³² is I; X³³ is V; X³⁴ is H; X³⁵ is I; and X⁵⁰ is H; or |
| | start position = aa position 490, 489, 492 or | X²⁹ is L; X³⁰ is F; X³¹ is I; X³² is I; X³³ is V; X³⁴ is R; X³⁵ is F; and X⁵⁰ is H; or |
| | 487 | X²⁹ is L; X³⁰ is F; X³¹ is I; X³² is I; X³³ is I; X³⁴ is R; X³⁵ is F; and X⁵⁰ is H; or |
| | end position = 513 | X²⁹ is L; X³⁰ is F; X³¹ is I; X³² is T; X³³ is V; X³⁴ is R; X³⁵ is F; and X⁵⁰ is H; or |
| | | X²⁹ is F; X³⁰ is F; X³¹ is I; X³² is I; X³³ is V; X³⁴ is R; X³⁵ is F and X⁵⁰ is H |
| 424 | | SWGYVX²⁹LIVX³⁰FCLX³¹IFVLX³²YX³³X³⁴X³⁵FX⁵⁰ |
| 425 | | YVX²⁹LIVX³⁰FCLX³¹IFVLX³²YX³³X³⁴X³⁵FX⁵⁰ |
| 426 | | FRSWGYVX²⁹LIVX³⁰FCLX³¹IFVLX³²YX³³X³⁴X³⁵FX⁵⁰ |
| 6 | Human HEMO intracellular domain | 513-563 from SEQ ID NO: 1 |
| 417 | start position 513, 510 or 514 | 510-563 from SEQ ID NO: 1 |
| 418 | end position 563 | 514-563 from SEQ ID NO: 1 |
| 411-413 | Generic sequence of intracellular domain for | X³⁸KSX³⁹RSX⁴⁰NSQX⁴¹LX⁴²X⁴³X⁴⁴LSPQQSA; or |
| | HEMO protein of Boreoeutheria | X³⁶X³⁷FX³⁸KSX³⁹RSX⁴⁰NSQX⁴¹LX⁴²X⁴³X⁴⁴LSPQQSA; or |
| | from HUM to SAK of FIGS. 11A-11B | KSX³⁹RSX⁴⁰NSQX⁴¹LX⁴²X⁴³X⁴⁴LSPQQSA; |
| | start position 513, 510 or 514 | wherein |
| | end position 563 | X³⁶ is R or H |
| | | X³⁷ is V or I or F |
| | | X³⁸ is R or G or H |
| | | X³⁹ is R or H |
| | | X⁴⁰ is L or F |
| | | X⁴¹ is P or T |
| | | X⁴² is N or Y or F |
| | | X⁴³ is L or P |
| | | X⁴⁴ is A or V |
| 414-416 | Generic sequence of intracellular domain for | X³⁸KSX³⁹RSX⁴⁰NSQX⁴¹LX⁴²X⁴³X⁴⁴LSPQQSAQX⁴⁵LX⁴⁶X⁴⁷ETSCQVSNRAMKX⁴⁸X⁴⁹TTHQYDTSLL; or |
| | HEMO protein of Boreoeutheria | X³⁶X³⁷FX³⁸KSX³⁹RSX⁴⁰NSQX⁴¹LX⁴²X⁴³X⁴⁴LSPQQSAQX⁴⁵LX⁴⁶X⁴⁷ETSCQVSNRAMKX⁴⁸X⁴⁹TTHQYDTSLL; |
| | from HUM to RHI of FIGS. 11A-11B | or |
| | start position 513, 510 or 514 | KSX³⁹RSX⁴⁰NSQX⁴¹LX⁴²X⁴³X⁴⁴LSPQQSAQX⁴⁵LX⁴⁶X⁴⁷ETSCQVSNRAMKX⁴⁸X⁴⁹TTHQYDTSLL; |
| | end position 563 | wherein |
| | | X³⁶ is R or H |
| | | X³⁷ is V or I or F |
| | | X³⁸ is R or G or H |
| | | X³⁹ is R or H |
| | | X⁴⁰ is L or F |
| | | X⁴¹ is P or T |
| | | X⁴² is N or Y or F |
| | | X⁴³ is L or P |
| | | X⁴⁴ is A or V |
| | | X⁴⁵ is L or Q |
| | | X⁴⁶ is L or I |
| | | X⁴⁷ is V or N |
| | | X⁴⁸ is G or E |
| | | X⁴⁹ is L or P |
| 8 | Example of fragment of human HEMO | 123-286 from SEQ ID NO: 1 |
| | ectodomain, which may be used for mAb | |
| | production (cf. example below) | |
| 150 | Shedding/cleavage region in the human | 380-480 from SEQ ID NO: 1 |
| 622 | HEMO protein | 380-420 from SEQ ID NO: 1 |
| 623 | | 428-438 from SEQ ID NO: 1 |
| 624 | | 450-480 from SEQ ID NO: 1 |
| 9-10 | Human HEMO major soluble ectodomain | 27-X¹ from SEQ ID NO: 1 wherein X¹ is aa position 432 or 433 |
| 183-184 | fragment (produced by shedding) | 25- X¹ from SEQ ID NO: 1 wherein X¹ is aa position 432 or 433 |
| 185-186 | End position 432 or 433 | 26- X¹ from SEQ ID NO: 1 wherein X¹ is aa position 432 or 433 |
| | Start position 27, 25 or 26 | |
| 11-12 | Human HEMO ectodomain fragment, which | X²-489 from SEQ ID NO: 1 wherein X² is aa position 433 or 434 |
| 189-190 | is retained on the cell surface after shedding | X²-488 from SEQ ID NO: 1 wherein X² is aa position 433 or 434 |
| 191-192 | of the major soluble fragment of SEQ ID NO: | X²-491 from SEQ ID NO: 1 wherein X² is aa position 433 or 434 |
| 193-194 | 9, 183 or 185 [SEQ ID NO: 11] or of SEQ ID | X²-486 from SEQ ID NO: 1 wherein X² is aa position 433 or 434 |
| | NO: 10, 184 or 186 [SEQ ID NO: 12] | |
| | Start position 433 or 434 | |
| | End position 489, 488, 491 or 486 | |
| 625-626 | Human HEMO membranar protein after | X²-563 from SEQ ID NO: 1 wherein X² is aa position 433 or 434 |
| | shedding of the major soluble fragment of | |
| | SEQ ID NO: 9, 183 or 185 [SEQ ID NO: 625] or | |
| | of SEQ ID NO: 10, 184 or 186 [SEQ ID NO: | |
| | 626] | |
| | Start position 433 or 434 | |
| | End position 563 | |
| 13-33 | Human HEMO secondary soluble | 27-X³ from SEQ ID NO: 1 wherein X³ = any aa position from among 380-420 |
| 670-689 | ectodomain fragment (produced by | |
| 195-215 | shedding) | 25-X³ from SEQ ID NO: 1 wherein X³ = any aa position from among 380-420 |
| 690-709 | start position 27, 25 or 26 | |
| 216-236 | end position at 380-420 | 26-X³ from SEQ ID NO: 1 wherein X³ = any aa position from among 380-420 |
| 710-729 | | |
| 34-54 | Human HEMO ectodomain fragment, which | X⁴-489 from SEQ ID NO: 1 wherein X⁴ = any aa position from among 381-421 |
| 730-749 | is retained on the cell surface after shedding | |
| | of the secondary soluble fragment of SEQ ID | |
| 237-257 | NO: 13-33, 195-215 or 216-236 | X⁴-488 from SEQ ID NO: 1 wherein X⁴ = any aa position from among 381-421 |
| 750-769 | start position at 381-421 | |
| 258-278 | end position 489, 488, 491 or 486 | X⁴-491 from SEQ ID NO: 1 wherein X⁴ = any aa position from among 381-421 |
| 770-789 | | |
| 279-299 | | X⁴-486 from SEQ ID NO: 1 wherein X⁴ = any aa position from among 381-421 |
| 790-809 | | |
| 627-647 | Human HEMO membranar protein after | X⁴-563 from SEQ ID NO: 1 wherein X⁴ = any aa position from among 381-421 |
| 810-829 | shedding of the secondary soluble fragment | |
| | of SEQ ID NO: 13-33, 195-215 or 216-236 | |
| | start position at 381-421 | |
| | end position 563 | |
| 55-75 | Alternative human HEMO secondary soluble | 27-X⁵ from SEQ ID NO: 1 wherein X⁵ = any aa position from among 450-480 |
| 830-839 | ectodomain fragment (produced by | |
| 300-320 | shedding) | 25-X⁵ from SEQ ID NO: 1 wherein X⁵ = any aa position from among 450-480 |
| 840-849 | start position 27, 25 or 26 | |
| 321-341 | end position at 450-480 | 26-X⁵ from SEQ ID NO: 1 wherein X⁵ = any aa position from among 450-480 |
| 850-859 | | |
| 76-96 | Human HEMO ectodomain fragment, which | X⁶-489 from SEQ ID NO: 1 wherein X⁶ = any aa position from among 451-481 |
| 860-869 | is retained on the cell surface after shedding | |
| 342-362 | of the alternative secondary soluble fragment | X⁶-488 from SEQ ID NO: 1 wherein X⁶ = any aa position from among 451-481 |
| 870-879 | of SEQ ID NO: 55-75, 300-320 or 321-341 | |
| 363-383 | start position at 451-481 | X⁶-491 from SEQ ID NO: 1 wherein X⁶ = any aa position from among 451-481 |
| 880-889 | end position 489, 488, 491 or 486 | |
| 384-404 | | X⁶-486 from SEQ ID NO: 1 wherein X⁶ = any aa position from among 451-481 |
| 890-899 | | |
| 648-668 | Human HEMO membranar protein after | X⁶-563 from SEQ ID NO: 1 wherein X⁶ = any aa position from among 451-481 |
| 900-909 | shedding of the alternative secondary soluble | |
| | fragment of SEQ ID NO: 55-75, 300-320 or | |
| | 321-341 | |
| | start position at 451-481 | |
| | end position 563 | |
| 991-1001 | Alternative human HEMO secondary soluble | 25-X⁷ from SEQ ID NO: 1 wherein X⁷ = any aa position from among 421-431 |
| 1002-1017 | ectodomain fragment (produced by | 25-X⁷ from SEQ ID NO: 1 wherein X⁷ = any aa position from among 434-449 |
| 1018-1028 | shedding) | 26-X⁷ from SEQ ID NO: 1 wherein X⁷ = any aa position from among 421-431 |
| 1029-1044 | start position 25, 26 or 27 | |
| | end position at 421-431 and 434-449 | 26-X⁷ from SEQ ID NO: 1 wherein X⁷ = any aa position from among 434-449 |
| 1045-1055 | | 27-X⁷ from SEQ ID NO: 1 wherein X⁷ = any aa position from among 421-431 |
| 1056-1071 | | 27-X⁷ from SEQ ID NO: 1 wherein X⁷ = any aa position from among 434-449 |
| 97-128 | Primers | cf. Table 4 |
| 152 | Nucleic acid coding for human HEMO protein | Coding for human HEMO of SEQ ID NO: 1 |
| | (CDS) | |
| 153-167 | Nucleic acid coding for non-human HEMO | Boreoeutheria mammals CPZ, GOR, ORA, GIB, MAC, BAB, AGM, COL, LAN, RHI, MAR, SQM, |
| | protein | SPI, SAK, CAT cf. FIGS. 11A, 11B, 12A, 12B |
| 669 | Human HEMO promoter | chr4: 52750959-52751715 757bp |
| 910 | Human HEMO ectodomain | 25-487 from SEQ ID NO: 1 |
| 911 | (without the signal peptide) | 25-490 from SEQ ID NO: 1 |
| 912 | start position 25, 26 or 27 | 25-492 from SEQ ID NO: 1 |
| 913 | end position 487, 490 or 492 | 26-487 from SEQ ID NO: 1 |
| 914 | | 26-490 from SEQ ID NO: 1 |
| 915 | | 26-492 from SEQ ID NO: 1 |
| 916 | | 27-487 from SEQ ID NO: 1 |
| 917 | | 27-490 from SEQ ID NO: 1 |
| 918 | | 27-492 from SEQ ID NO: 1 |
| 919 | HTM5 | 387-471 from SEQ ID NO: 1 |
| 920 | Streptag-HTM5 peptide | (StrepTaglinker(GGGS)x3)-StrepTag)-HTM5 |
| 921 | SU-HEMO | 1-351 from SEQ ID NO: 1 |
| 922 | TM-HEMO | internal deletion from aa 34 to 352 = 1-33 + 353-563 from SEQ ID NO: 1 |
| 923 | post-SHED HEMO | internal deletion from aa 34 to 432 = 1-33 + 433-563 from SEQ ID NO: 1 |
| 924-939 | human HEMO ectodomain fragments | see Table 7 |
| 981-987 | | |
| 988 | HST5 | 280-400 from SEQ ID NO: 1 |
| 989 | Streptag-HST5 peptide | (StrepTaglinker(GGGS)x3)-StrepTag)-HST5 |
| 990 | 57 amino acids of the ectodomain part of the | 433-489 from SEQ ID NO: 1 (see example 3) |
| | HEMO protein | |
| 940 | Cat HEMO signal peptide | 1-22 from SEQ ID NO: 143 |
| 941 | start position 1 | 1-23 from SEQ ID NO: 143 |
| 942 | end position 22, 23, 24, 25 or 26 | 1-24 from SEQ ID NO: 143 |
| 943 | | 1-25 from SEQ ID NO: 143 |
| 944 | | 1-26 from SEQ ID NO: 143 |
| 945 | Cat HEMO ectodomain | 23-485 from SEQ ID NO: 143 |
| 946 | start position 23, 24, 25, 26 or 27 | 23-486 from SEQ ID NO: 143 |
| 947 | end position 485, 486, 487, 488, 489, 490 or | 23-487 from SEQ ID NO: 143 |
| 948 | 491 | 23-488 from SEQ ID NO: 143 |
| 949 | | 23-489 from SEQ ID NO: 143 |
| 950 | | 23-490 from SEQ ID NO: 143 |
| 951 | | 23-491 from SEQ ID NO: 143 |
| 952 | | 24-485 from SEQ ID NO: 143 |
| 953 | | 24-486 from SEQ ID NO: 143 |
| 954 | | 24-487 from SEQ ID NO: 143 |
| 955 | | 24-488 from SEQ ID NO: 143 |
| 956 | | 24-489 from SEQ ID NO: 143 |
| 957 | | 24-490 from SEQ ID NO: 143 |
| 958 | | 24-491 from SEQ ID NO: 143 |
| 606 | Cat HEMO ectodomain (continued) | 25-485 from SEQ ID NO: 143 |
| 959 | start position 23, 24, 25, 26 or 27 | 25-486 from SEQ ID NO: 143 |
| 960 | end position 485, 486, 487, 488, 489, 490 or | 25-487 from SEQ ID NO: 143 |
| 516 | 491 | 25-488 from SEQ ID NO: 143 |
| 471 | | 25-489 from SEQ ID NO: 143 |
| 961 | | 25-490 from SEQ ID NO: 143 |
| 561 | | 25-491 from SEQ ID NO: 143 |
| 621 | | 26-485 from SEQ ID NO: 143 |
| 962 | | 26-486 from SEQ ID NO: 143 |
| 963 | | 26-487 from SEQ ID NO: 143 |
| 531 | | 26-488 from SEQ ID NO: 143 |
| 486 | | 26-489 from SEQ ID NO: 143 |
| 964 | | 26-490 from SEQ ID NO: 143 |
| 576 | | 26-491 from SEQ ID NO: 143 |
| 591 | | 27-485 from SEQ ID NO: 143 |
| 965 | | 27-486 from SEQ ID NO: 143 |
| 966 | | 27-487 from SEQ ID NO: 143 |
| 501 | | 27-488 from SEQ ID NO: 143 |
| 456 | | 27-489 from SEQ ID NO: 143 |
| 967 | Cat HEMO ectodomain (continued and end) | 27-490 from SEQ ID NO: 143 |
| 546 | start position 23, 24, 25, 26 or 27 | 27-491 from SEQ ID NO: 143 |
| | end position 485, 486, 487, 488, 489, 490 or | |
| | 491 | |
| 968 | Cat CWLC motif | 44-47 from SEQ ID NO: 143 |
| 969 | Cat Furin motif | 352-355 from SEQ ID NO: 143 |
| 970 | Cat ImmunoSuppresive Domaine (ISD) | 418-433 from SEQ ID NO: 143 |
| 971 | Cat HEMO C-X6-CC motif | 434-442 from SEQ ID NO: 143 |
| 972 | Cat HEMO transmembrane domain | 486-510 from SEQ ID NO: 143 |
| 973 | start position 486, 487, 488, 489, 490, 491 or | 487-510 from SEQ ID NO: 143 |
| 974 | 492 | 488-510 from SEQ ID NO: 143 |
| 975 | end position 510 | 489-510 from SEQ ID NO: 143 |
| 976 | | 490-510 from SEQ ID NO: 143 |
| 977 | | 491-510 from SEQ ID NO: 143 |
| 978 | | 492-510 from SEQ ID NO: 143 |
| 979 | Cat HEMO intracellular domain | 511-578 from SEQ ID NO: 143 |
| | start position 511 | |
| | end position 578 | |
| 980 | Cat HEMO Shedding region | 378-479 from SEQ ID NO: 143 |
| | start position 378 | |
| | end position 479 | |

### BIBLIOGRAPHIC REFERENCES

Bischof P, Irminger-Finger I (2005) The human cytotrophoblastic cell, a mononuclear chameleon. Int J Biochem Cell Biol 37(1):1-16.
Blaise S, de Parseval N, Benit L, Heidmann T (2003) Genomewide screening for fusogenic human endogenous retrovirus envelopes identifies syncytin 2, a gene conserved on primate evolution. Proc Natl Acad Sci USA. 100(22):13013-8.
Blond J L, Lavillette D, Cheynet V, Bouton O, Oriol G, Chapel-Fernandes S, Mandrand B, Mallet F, Cosset F L (2000) An envelope glycoprotein of the human endogenous retrovirus HERV-W is expressed in the human placenta and fuses cells expressing the type D mammalian retrovirus receptor. J Virol. 74(7):3321-9.
Cho K, Shih L (2009) Ovarian Cancer. Annu Rev Pathol 1(4):287-313.
Cole L A (2009) New discoveries on the biology and detection of human chorionic gonadotropin. Reprod Biol Endocrinol. 7:8.
Cornelis G, Vernochet C, Carradec Q, Souquere S, Mulot B, Catzeflis F, Nilsson M A, Menzies B R, Renfree M B, Pierron G, Zeller U, Heidmann O, Dupressoir A, Heidmann T (2015) Retroviral envelope gene captures and syncytin exaptation for placentation in marsupials. Proc Natl Acad Sci USA 112(5):E487-96.
Deaton A, Bird A (2011) CpG islands and the regulation of transcription. Genes Dev 24(10):1010-22.
Denner J. (2016) Expression and function of endogenous retroviruses in the placenta. Apmis 124(1-2):31-43.
Dolnik O, Volchkova V, Garten W, Carbonnelle C, Becker S, Kahnt J, Stroller U, Klenk H D, Volchkov V (2004) Ectodomain shedding of the glycoprotein GP of Ebola virus. Embo J 23(10):2175-84.
Esnault C, Cornelis G, Heidmann O, Heidmann T (2013) Differential Evolutionary Fate of an Ancestral Primate Endogenous Retrovirus Envelope Gene, the EnvV Syncytin, Captured for a Function in Placentation. PLoS Genet 9(3):1-12.
Friedli M, Turelli P, Kapopoulou A, Rauwel B, Castro-d N, Rowe H M, Ecco G, Unzu C, Planet E, Lombardo A, Mangeat B, Wildhaber B E, Naldini L, Trono D (2014) Loss of transcriptional control over endogenous retroelements during reprogramming to pluripotency Genome Res 24(8):1251-59.
Gautier L, Cope L, Bolstad B M, Irizarry R A (2004) Affy—Analysis of Affymetrix GeneChip data at the probe level. Bioinformatics 20(3):307-15.
Henzy J E, Johnson W E (2013) Pushing the endogenous envelope. Philos Trans R Soc Lond B Biol Sci. 368(1626):20120506.
Jurka J, Kapitonov V V, Pavlicek A, Klonowski P, Kohany O, Walichiewicz J (2005) Repbase Update, a database of eukaryotic repetitive elements. Cytogenet Genome Res. 110(1-4):462-7.
Kurman R J, Shih I-M (2016) The Dualistic Model of Ovarian Carcinogenesis. Am J Pathol 186(4):733-47.
Lavialle C, Cornelis G, Dupressoir A, Esnault C, Heidmann O, Vernochet C, Heidmann T (2013) Paleovirology of “syncytins”, retroviral env genes exapted for a role in placentation. Philos Trans R Soc L B Biol Sci. 368(1626):20120507.
Lukk M, Kapushesky M, Nikkila J, Parkinson H, Goncalves A, Huber W, Ukkonen E, Brazma A (2010) A global map of human gene expression. Nat Biotechnol 28(4):322-4.
Maltepe E, Fisher S J (2015) Placenta: the forgotten organ. Annu Rev Cell Dev Biol 31(1):523-52.
Mangeney M, Renard M, Schlecht-Louf G, Bouallaga I, Heidmann O, Letzelter C, Richaud I, Ducos B, Heidmann T (2007) Placental syncytins: Genetic disjunction between the fusogenic and immunosuppressive activity of retroviral envelope proteins. Proc Natl Acad Sci USA 104(51):20534-9.
Mi S, Lee X, Li X, Veldman G M, Finnerty H, Racie L, LaVallie E, Tang X Y, Edouard P, Howes S, Keith J C Jr, McCoy J M (2000) Syncytin is a captive retroviral envelope protein involved in human placental morphogenesis. Nature 403 (February):785-9.
Okazaki I, Nabeshima K (2012) Introduction: MMPs, ADAMs/ADAMTSs research products to achieve big dream. Anticancer Agents Med Chem 12(7):688-706.
de Parseval N, Lazar V, Casella J-F, Benit L, Heidmann T. Survey of human genes of retroviral origin: identification and transcriptome of the genes with coding capacity for complete envelope proteins. J Virol 2003; 77(19):10414-22.
Taminau J, Meganck S, Lazar C, Steenhoff D, Coletta a, Molter C, Duque R, de Schaetzen V, Weiss Solis D Y, Bersini H, Nowe A (2012) Unlocking the potential of publicly available microarray data using inSilicoDb and inSilicoMerging R/Bioconductor packages. BMC Bioinformatics 13:335.
Toth G, Jurka J (1994) Repetitive DNA in and around translocation breakpoints of the Philadelphia chromosome. Gene 140(2):285-8.
Uhlén M, Fagerberg L, Hallstrom B M, Lindskog C, Oksvold P, Mardinoglu A, Sivertsson A, Kampf C, Sjostedt E, Asplund A, Olsson I, Edlund K, Lundberg E, Navani S, Szigyarto C A, Odeberg J, Djureinovic D, Takanen J O, Hober S, Alm T, Edqvist P H, Berling H, Tegel H, Mulder J, Rockberg J, Nilsson P, Schwenk J M, Hamsten M, von Feilitzen K, Forsberg M, Persson L, Johansson F, Zwahlen M, von Heijne G, Nielsen J, Pontén F (2015) Tissue-based map of the human proteome. Science 347(6220):1260419.
Vargiu L, Rodriguez-Tome P, Sperber G O, Cadeddu M, Grandi N, Blikstad V, Tramontano E, Blomberg J (2016) Classification and characterization of human endogenous retroviruses; mosaic forms are common. Retrovirology 13(1):7.
Villesen P, Aagaard L, Wiuf C, Pedersen F S (2004) Identification of endogenous retroviral reading frames in the human genome. Retrovirology 1:32.
Weber S, Saftig P (2012) Ectodomain shedding and ADAMs in development. Development 139(20):3693-709.
Xue Z, Huang K, Cai C, Cai L, Jiang C Y, Feng Y, Liu Z, Zeng Q, Cheng L, Sun Y E, Liu J Y, Horvath S, Fan G (2013) Genetic programs in human and mouse early embryos revealed by single-cell RNA sequencing. Nature 500(7464):593-7.
Yan L, Yang M, Guo H, Yang L, Wu J, Li R, Liu P, Lian Y, Zheng X, Yan J, Huang J, Li M, Wu X, Wen L, Lao K, Li R, Qiao J, Tang F (2013) Single-cell RNA-Seq profiling of human preimplantation embryos and embryonic stem cells. Nat Struct Mol Biol 20(9):1131-9.

1. A polypeptide consisting of a N-terminal fragment of the ectodomain of an endogenous Boreoeutheria retroviral Env protein,
a. wherein said retroviral Env protein is
the amino acid sequence of SEQ ID NO: 1; or
the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1; or
a sequence chosen from among the sequences of SEQ ID NOs: 129-143,
b. wherein said ectodomain, without its signal peptide or fragment thereof, of said retroviral Env protein consists of:
443-468 or 443-467 amino acids of said sequences which more particularly comprise in N-term to C-term orientation
i. a CWLC amino acid sequence,
ii. an amino acid sequence chosen from among the CTQG sequence, the CTQR sequence, the CIQR sequence, the RTQR sequence and the RTKR sequence, and
iv. an amino acid sequence of SEQ ID NO: 149;
wherein the ectodomain of said retroviral Env protein consists more particularly of a sequence chosen from among the sequences of SEQ ID NOs: 178, 912 and 547-561, and the sequences, which are the fragments of at least one of the sequences of SEQ ID NOs: 172, 911 and 457-471, and which differ by at most 7 or 8 amino acids in length of at least one of the sequences of SEQ ID NOs: 178, 912 and 547-561,
wherein the sequence of said N-terminal fragment:
consists of a number of amino acids lower than said ectodomain, said lower number being chosen from among 344-457 or 374-446,
starts at the N-terminal extremity of said ectodomain; and
comprises said sequence of b.i. and said sequence of b.ii.

2. The polypeptide consisting of a N-terminal fragment of the ectodomain of an endogenous Boreoeutheria retroviral Env protein according to claim 1,
a. wherein said retroviral Env protein is
the amino acid sequence of SEQ ID NO: 1; or
the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1; or
a sequence chosen from among the sequences of SEQ ID NOs: 129-142,
b. wherein said ectodomain, without its signal peptide or fragment thereof, of said retroviral Env protein consists of:
443-468 or 443-467 amino acids of said sequences which more particularly comprise in N-term to C-term orientation
i. a CWLC amino acid sequence,
ii. an amino acid sequence chosen from among the CTQG sequence, the CTQR sequence, the CIQR sequence and the RTKR sequence,
iii. an amino acid sequence of SEQ ID NO: 148, and
iv. an amino acid sequence of SEQ ID NO: 149;
wherein the ectodomain of said retroviral Env protein consists more particularly of a sequence chosen from among the sequences of SEQ ID NOs: 178, 912 and 547-560, and the sequences, which are the fragments of at least one of the sequences of SEQ ID NOs: 172, 911 and 457-470, and which differ by at most 7 or 8 amino acids in length of at least one of the sequences of SEQ ID NOs: 178, 912 and 547-560,
wherein the sequence of said N-terminal fragment:
consists of a number of amino acids lower than said ectodomain, said lower number being chosen from among 344-457 or 374-446,
starts at the N-terminal extremity of said ectodomain; and
comprises said sequence of b.i. and said sequence of b.ii.

3. The polypeptide consisting of a N-terminal fragment of the ectodomain of an endogenous cat retroviral Env protein according to claim 1,
a. wherein said retroviral Env protein is
the amino acid sequence of SEQ ID NO: 143; or
the amino acid sequence which is at least 80% identical to SEQ ID NO: 143, more particularly the amino acid sequence which is at least 81%, 82%, 83%, 84%, 85%, 86%, 87%, 88%, 89%, 90%, 91%, 92%, 93%, 94%, 95%, 96%, 97%, 98% or 99% identical to SEQ ID NO: 143,
b. wherein said ectodomain, without its signal peptide or fragment thereof, of said retroviral Env protein consists of 443-468 or 443-467 amino acids of said sequences which more particularly comprise in N-term to C-term orientation
i. a CWLC amino acid sequence,
ii. a RTQR sequence, and
iv. an amino acid sequence of SEQ ID NO: 149;
wherein the ectodomain of said retroviral Env protein consists more particularly of the sequences of SEQ ID NOs: 561 and 951, and the sequences, which are the fragments of the sequences of SEQ ID NOs: 471 and 949, and which differ by at most 7 or 8 amino acids in length of at least one of the sequences of SEQ ID NOs: 561 and 951,
wherein the sequence of said N-terminal fragment:
consists of a number of amino acids lower than said ectodomain, said lower number being chosen from among 352-457 or 374-446;
starts at the N-terminal extremity of said ectodomain; and
comprises said sequence of b.i. and said sequence of b.ii.

4. The polypeptide consisting of a N-terminal fragment of the ectodomain of an endogenous Boreoeutheria retroviral Env protein according to claim 1,
a. wherein said retroviral Env protein is
the amino acid sequence of SEQ ID NO: 1; or
the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1; or
a sequence chosen from among the sequences of SEQ ID NOs: 129-142,
b. wherein said ectodomain, without its signal peptide or fragment thereof, of said retroviral Env protein consists of:
443-468 or 443-467 amino acids of said sequences which more particularly comprise in N-term to C-term orientation
i. a CWLC amino acid sequence,
ii. an amino acid sequence chosen from among the CTQG sequence, the CTQR sequence, the CIQR sequence and the RTKR sequence,
iii. an amino acid sequence of SEQ ID NO: 148, and
iv. an amino acid sequence of SEQ ID NO: 149;
wherein the ectodomain of said retroviral Env protein consists more particularly of a sequence chosen from among the sequences of SEQ ID NOs: 178, 912 and 547-560, and the sequences, which are the fragments of at least one of the sequences of SEQ ID NOs: 172, 911 and 457-470, and which differ by at most 7 or 8 amino acids in length of at least one of the sequences of SEQ ID NOs: 178, 912 and 547-560,
wherein the sequence of said N-terminal fragment:
consists of a number of amino acids lower than said ectodomain, said lower number being chosen from among 344-457 or 374-446,
starts at the N-terminal extremity of said ectodomain; and
comprises said sequence of b.i. and said sequence of b.ii,
wherein the sequence of said N-terminal fragment consists of a number of amino acids lower than said ectodomain, said lower number being chosen from among 354-456 or 374-446.

5. The polypeptide consisting of a N-terminal fragment of the ectodomain of an endogenous Boreoeutheria retroviral Env protein according to claim 1,
a. wherein said retroviral Env protein is
the amino acid sequence of SEQ ID NO: 1; or
the amino acid sequence which is at least 59% identical to SEQ ID NO: 1, more particularly the amino acid sequence which is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 1; or
a sequence chosen from among the sequences of SEQ ID NOs: 129-142,
b. wherein said ectodomain, without its signal peptide or fragment thereof, of said retroviral Env protein consists of:
443-468 or 443-467 amino acids of said sequences which more particularly comprise in N-term to C-term orientation
i. a CWLC amino acid sequence,
ii. an amino acid sequence chosen from among the CTQG sequence, the CTQR sequence, the CIQR sequence and the RTKR sequence,
iii. an amino acid sequence of SEQ ID NO: 148, and
iv. an amino acid sequence of SEQ ID NO: 149;
wherein the ectodomain of said retroviral Env protein consists more particularly of a sequence chosen from among the sequences of SEQ ID NOs: 178, 912 and 547-560, and the sequences, which are the fragments of at least one of the sequences of SEQ ID NOs: 172, 911 and 457-470, and which differ by at most 7 or 8 amino acids in length of at least one of the sequences of SEQ ID NOs: 178, 912 and 547-560,
wherein the sequence of said N-terminal fragment:
consists of a number of amino acids lower than said ectodomain, said lower number being chosen from among 344-457 or 374-446,
starts at the N-terminal extremity of said ectodomain; and
comprises said sequence of b.i. and said sequence of b.ii,
wherein
a. the C-terminal extremity of said N-terminal fragment is located from amino acid at position 380 to amino acid at position 480 of the sequences SEQ ID NOs: 1, 129-134 and 136-137 and before the N-terminal extremity of the transmembrane domain of the sequences SEQ ID NOs: 1, 129-134 and 136-137 at a location of 6 to 112 amino acids upstream the N-terminal extremity of the transmembrane domain of the sequences SEQ ID NOs: 1, 129-134 and 136-137; or
b. the C-terminal extremity of said N-terminal fragment is located from amino acid at position 379 to amino acid at position 479 of the sequence SEQ ID NO: 135 and before the N-terminal extremity of the transmembrane domain of the sequence SEQ ID NO: 135 at a location of 6 to 112 amino acids upstream the N-terminal extremity of the transmembrane domain of the sequence SEQ ID NO: 135; or
c. the C-terminal extremity of said N-terminal fragment is located from amino acid at position 380 to amino acid at position 479 of the sequence SEQ ID NO: 138 and before the N-terminal extremity of the transmembrane domain of the sequence SEQ ID NO: 138 at a location of 6 to 111 amino acids upstream the N-terminal extremity of the transmembrane domain of the sequence SEQ ID NO: 138; or
d. the C-terminal extremity of said N-terminal fragment is located from amino acid at position 380 to amino acid at position 476 of the sequences SEQ ID NOs: 139-140 and 142 and before the N-terminal extremity of the transmembrane domain of the sequences SEQ ID NOs: 139-140 and 142 at a location of 3 to 105 amino acids upstream the N-terminal extremity of the transmembrane domain of the sequences SEQ ID NOs: 139-140 and 142; or
e. the C-terminal extremity of said N-terminal fragment is located from amino acid at position 370 to amino acid at position 466 of the sequence SEQ ID NO: 141 and before the N-terminal extremity of the transmembrane domain of the sequence SEQ ID NO: 141 at a location of 3 to 105 amino acids upstream the N-terminal extremity of the transmembrane domain of the sequence SEQ ID NO: 141.

6. The polypeptide consisting of a N-terminal fragment of the ectodomain of an endogenous human retroviral Env protein according to claim 1,
a. wherein said human retroviral Env protein is
the amino acid sequence of SEQ ID NO: 1; or
the amino acid sequence which is at least 80% identical to SEQ ID NO: 1, more particularly the amino acid sequence which is at least 81%, 82%, 83%, 84%, 85%, 86%, 87%, 88%, 89%, 90%, 91%, 92%, 93%, 94%, 95%, 96%, 97%, 98% or 99% identical to SEQ ID NO: 1,
b. wherein said ectodomain, without its signal peptide or fragment thereof, of said retroviral Env protein consists of:
443-468 or 443-467 amino acids of said sequences which more particularly comprise in N-term to C-term orientation
i. a CWLC amino acid sequence,
ii. an amino acid sequence chosen from among the CTQG sequence, the CTQR sequence, the CIQR sequence and the RTKR sequence,
iii. an amino acid sequence of SEQ ID NO: 148, and
iv. an amino acid sequence of SEQ ID NO: 149;
wherein the ectodomain of said human retroviral Env protein consists more particularly of:
a sequence chosen from among the sequences of SEQ ID NOs: 178 and 912 and the sequences, which are the fragments of at least one of the sequences of SEQ ID NOs: 172 and 911, and which differ by at most 7 or 8 amino acids in length of at least one of the sequences of SEQ ID NO: 178 and 912,
wherein the sequence of said N-terminal fragment:
consists of a number of amino acids lower than said ectodomain, said lower number being chosen from among 354-456 or 374-446,
starts at the N-terminal extremity of said ectodomain;
comprises said sequence of b.i. and said sequence of b.ii.; and
the C-terminal extremity of said N-terminal fragment is located from amino acid at position 380 to amino acid at position 480 of the sequence SEQ ID NO: 1 and before the N-terminal extremity of the transmembrane domain of the sequence SEQ ID NO: 1 at a location of 6 to 112 amino acids upstream the N-terminal extremity of the transmembrane domain of the sequence SEQ ID NO: 1.

7. The polypeptide of claim 1, wherein the sequence of the N-terminal fragment of said ectodomain comprises a fragment of, but not a full-length sequence of, an amino acid sequence of SEQ ID NO: 148;
more particularly wherein the sequence of the N-terminal fragment of said ectodomain comprises or consists of a sequence chosen from among the sequences of SEQ ID NOs: 9-10, 183-184 and 185-186.

8. The polypeptide of claim 1,
a. wherein the sequence of the N-terminal fragment of said ectodomain does not comprise the sequence of an amino acid sequence of SEQ ID NO: 148, and does not comprise any fragment of an amino acid sequence of SEQ ID NO: 148,
more particularly wherein the sequence of the N-terminal fragment of said ectodomain comprises or consists of a sequence chosen from among the sequences of SEQ ID NO: 13-33, 670-689, 195-215, 690-709, 216-236 and 710-729;
or
b. wherein the sequence of the N-terminal fragment of said ectodomain comprises the sequence of an amino acid sequence of SEQ ID NO: 148,
more particularly wherein the sequence of the N-terminal fragment of said ectodomain comprises or consists of a sequence chosen from among the sequences of SEQ ID NOs: 55-75, 830-839, 300-320, 840-849, 321-341 and 850-859.

9. The polypeptide of claim 1,
wherein the sequence of the N-terminal fragment of said ectodomain comprises or consists of a sequence chosen from among the sequences of SEQ ID NOs: 9-10, 184-186 and 991-1071.

10. A product, wherein said product is:
a. an antibody, or
b. a monoclonal antibody, more particularly the monoclonal antibody which is produced by the hybridoma deposited at the CNCM under accession number I-5211, or
c. a Fab, Fab′ or F(ab)2 fragment, or
d. a scFv, or
e. a sdAb, or
f. the variable domain of a sdAb,
and wherein said product is optionally linked to at least one drug, and wherein said product specifically binds to the polypeptide of claim 1.

11. A kit which comprises a product, wherein said product is used in at least one of the following five uses:
a. the detection of the appearance of a tumor or the following of the evolution of a tumor in a subject, wherein said subject is a Boreoeutheria;
and/or
b. the determination of the histotype, grade or stage of a tumor, wherein said cancer is an ovarian cancer, an uterine cancer, a cervical cancer, a gestational cancer, a breast cancer, a lung cancer, a stomach cancer, a colon cancer, a liver cancer, a kidney cancer, a prostate cancer, an urothelial cancer, a germ cell cancer, a brain cancer, a head and neck cancer, a pancreatic cancer, a thyroid cancer, a thymus cancer, a skin cancer, a bone cancer or a bone marrow cancer;
and/or
c. the detection of a defect in the placentation of a pregnant subject, more particularly a placentation defect placing said pregnant subject at risk of placental abruption, pre-eclampsia or eclampsia;
and/or
d. the purification or isolation of circulating cells of a Boreoeutheria, more particularly for purifying or isolating Boreoeutheria circulating cells, which are tumor cells, or tumor stem cells, or placental cells;
and/or
e. the purification or isolation of non-circulating cells of a Boreoeutheria, more particularly for purifying or isolating non-circulating cells in a fresh tumor or biopsy sample from a Boreoeutheria, and
wherein said product is the product of claim 10, and wherein said product is optionally linked or bound to a detection label.
